SEQUENCE LISTING (3 sequences)

SEQ ID NO: 1 - RNA, 6240 nt, Homo sapiens
  source 1..6240: mol_type = mRNA
agatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 2 - RNA, 2581 nt, Homo sapiens
  source 1..2581: mol_type = mRNA
gcaccctggtcatctgcggactcagcctgagcttccagagggcctaggagcagtaagggagtgagtgggcaactcggcgcatgaaggaggccgccctcatctgcctggcaccctctgtacccccgatcttgacggtgaagtcctgggacaccatgcagttgcgggctgctagatctcggtgcacaaacttgttggcagcaagctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttgaaaaaaaaaa

SEQ ID NO: 3 - RNA, 4134 nt, Homo sapiens
  source 1..4134: mol_type = mRNA
gcacttgtacatttctgcagccgcgcggcgagccattcgcggcggctgctgcagctcctactgcatcttccttctcttcctttcctcgggctccggtctcggagtcggagagcgcgcctcgcttccagagcccccggacccggcgagtcagcgatcgccgagccggccaccatgcccggcagaccgcgccactaggcgctcctcgcggctcccacccggcggcggcggcggcggcggcggcgtccgcgatggtttcagacgctgaaggattttgcatctgatcgctcggcgtttcaaagaagcagcgatcggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccagtatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaacgctgatggctcccagcttcctctcttccgcatgaacatcagtcagtgtgaccttcctgagatcagcgtgagccacgtcaacctgaccgtacgagagggtgacaacgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtgggtcttttcaaacatagacaatcatgggatattaaacttgaaggacaatagagatcatctagtcccatcaactcactatatatatgaggaacctgaggtccagagtggggaagtgtcttacccaaggtcacatggtttcagagaaattatgttgaatccaataagccttcccggacattccaagcctcttaaccatggcatctatgttgaggatgtcaatgtttatttcagcaaaggacgtcatggcttttaaaaactccttttaagcctccttgttttgatgtcaccttggtaggctgggccctctgagaggttggaagctctaggcattgttctctttggatccagggatgctaagtagaaactgcatgagccaccagtgccccggcaccctttaacaccaccagatgggtgttttcccccatccaccactggcagggttgccccttccctccaatcatcactgtgctccttttttcccggcctacgaggcagctcctgccactatctttagagccaataaagagaattaaaaacctgtgcaccaggagcatcttttaaatacactagccattctcttgctttacaaaaacaacctaaccatcacaagaaagcctgatgaagtccagccgtgctccagcctcactttccctgcttggaagcgtggggtctccctggctctcccaggataccatgctgtcctcttagtgacctcgtcgccctgcaacctccagtggggaagagtcacagagagcacctaagcagaggtggagacggcgcggtaagaggagggggagccaggctcaagtattggcaccaagttaggtctcagaggaaagaatggaaaccaatcactttacatttttatttttattttcggtggaaaaatcatccttttttgggacatacttgccccctacttcctcttctctctggaacggctcacaatgagtgtgacattagaaaactccttgcagaggagagtttctccaggctcttcctgggcccttagatctgcagttccgacaagctttggctgcaggaggttttacccatgaactggccatcctactaggaccacaagggaccaagggaatcagggacaaaggcccttcctgccagcccatgatcccgggattggctctcttcccctacttccacttattcttgactctgagaacttttggaacccaatggaatcagcatttcaaggtcaagatgaactgaaggggaagagaagtaaaacttggcctcctccagcccctctcatggcaccaatggaagtgtcctcctgttctctggtcaatatgtgtgtttactttgcttgctttgactcatgccttactccatggccaccctctcccaaagagggggctcgcttcccccattttcaacttgatccactgaggagagggaagggggtgactttcccttcttcagtaggaaaggcacatttgtagggcctgaaactctcccgtatttgctgactcattggtggagtagacttctggctcccagctccactggcccatggggcctccattgtatgaagtcagcataggctgcccacctaatggtggagagcatgaaactgggagcatcctgtggggggcttgtgggggaaaaaggtggttgttttaacccaccgttgttttggggtggtgttgcacactagtagagaatagagtctatgcctttggcaaatttaactgggagtttggattcccacttaagggttttacttcttgggtcctgtggatggtggttcttcgtgtcaggatcccagcccgattctgcaaatgcctccatggggtttaaaaacatgaggctttccaagttcttgcccagtatctggggcagcctccagagtatcacctgggagttcaggttctctccagggctccaggtgtgtgtttatctcgccccctccagctctcctcatcctgctccccattgctccatgtcaggctgttccccattgtgccctgctgatgctttgggtccagggcctcctcccaagtgtggctttaaggagtaagcttgaggatgatgttttttaattattgtaaatcattacctcatttccagcctcccaggctccatccatcccagcatcttttattctgccattttcctcaccttgtgctatgacaatggggcgttgtgtttccacagagacttataggagtgttcagtgtatagtttcttaataaacactttattttctaatgaaa

(19) United States
(12) Patent Application Publication
(10) Pub. No.: US 2025/0129429 A1
(43) Pub. Date: Apr. 24, 2025

(54) GENE FUSIONS IN SARCOMA

(71) Applicant: Foundation Medicine, Inc., Boston, MA (US)

(72) Inventors: Russell MADISON, Encinitas, CA (US); Geoffrey R. OXNARD, Arlington, MA (US); Alexa SCHROCK, Okemos, MI (US); Alex KESSLER, Goshen, MA (US)

(73) Assignee: Foundation Medicine, Inc., Boston, MA (US)

(21) Appl. No.: 18/689,774

(22) PCT Filed: Sep. 9, 2022
(86) PCT No.: PCT/US2022/076227

§ 371 Date: Mar. 6, 2024

Related U.S. Application Data
(60) Provisional application No. 63/242,883, filed on Sep. 10, 2021.

(51) Int. Cl. C12Q 1/6886; G01N 33/574

(52) U.S. Cl. CPC C12Q 1/6886; G01N 33/57484; C12Q 2600/156

(57) ABSTRACT

Provided herein are kinase fusion nucleic acid molecules and polypeptides, methods related to detecting kinase fusion nucleic acid molecules and polypeptides in cancer, as well as methods of treatment and uses related thereto. Detection of a kinase fusion nucleic acid molecule or the fusion polypeptide encoded by the fusion nucleic acid molecule can be used to identify individuals that may benefit from treatment with an anti-cancer therapy.

### CROSS-REFERENCE TO RELATED APPLICATIONS

This application claims the benefit of U.S. Provisional Application No. 63/242,883, filed Sep. 10, 2021, which is hereby incorporated by reference in its entirety.

### REFERENCE TO AN ELECTRONIC SEQUENCE LISTING

The content of the electronic sequence listing (197102007840SEQLIST.xml; Size: 16,510 bytes; and Date of Creation: Sep. 6, 2022) is herein incorporated by reference in its entirety.

### TECHNICAL FIELD

Provided herein are kinase fusion nucleic acid molecules and polypeptides, methods related to detecting such kinase fusion nucleic acid molecules and polypeptides, as well as methods of diagnosis/treatment and uses related thereto.

### BACKGROUND

Kinases activated by gene fusions are established oncogenic drivers and therapeutic targets, and have been associated with both hematopoietic malignancies and solid tumors. For example, a number of tyrosine kinase gene fusions (e.g., of the NTRK family) have been identified across several cancers. Recently, approvals of NTRK inhibitors have led to routine diagnostic testing for NTRK fusions across many cancer types (Cocco et al. (2018) Nat Rev Clin Oncol, 15:731-747). The anaplastic lymphoma receptor tyrosine kinase (ALK) gene is a known oncogene that has been associated with cancerous phenotypes, including inflammatory myofibroblastic tumors, neuroblastoma, lung cancer, non-Hodgkin's lymphoma, and anaplastic large cell lymphoma, among others. Chromosomal rearrangements involving the ALK gene have been found in certain cancers. For example, a chromosomal rearrangement that generates a fusion gene resulting in the juxtaposition of the N-terminal region of nucleophosmin (NPM) with the kinase domain of ALK is known to be associated with non-Hodgkin's lymphoma (Morris, SW (1994) Science 263: 1281-1284).

Kinase fusions have also been observed in patients following initial treatment with targeted therapies, suggesting that kinase fusions may be an acquired resistance (AR) mechanism, and that patients with such fusions could benefit from strategies that target the acquired kinase fusion. See, e.g, Xu et al., Cancer Manag Res (2019) 11:6343-51; Piotrowska et al., Cancer Discov (2018) 8(12):1529-39; Schrock et al., J Thorac Oncol (2018) 13(9):1312-23; and Schrock et al., J Thorac Oncol 2019; 14(2):255-64).

Thus, there is a need in the art for characterizing the pan-cancer landscape of kinase fusions, and for developing methods, compositions, and assays for evaluating and treating patients with such fusions, e.g., patients with a sarcoma of the present disclosure.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In one aspect, provided herein is a method of identifying an individual having a cancer who may benefit from a treatment comprising an anti-cancer therapy, the method comprising detecting in a sample from the individual a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; wherein detection of the fusion nucleic acid molecule or the fusion polypeptide encoded by the fusion nucleic acid molecule in the sample identifies the individual as one who may benefit from the treatment comprising the anti-cancer therapy.

In another aspect, provided herein is a method of selecting a treatment for an individual having a cancer, the method comprising detecting in a sample from the individual a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; wherein detection of the fusion nucleic acid molecule or the fusion polypeptide encoded by the fusion nucleic acid molecule in the sample identifies the individual as one who may benefit from the treatment comprising an anti-cancer therapy.

In another aspect, provided herein is a method of identifying one or more treatment options for an individual having a cancer, the method comprising: detecting in a sample from the individual a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; and generating a report comprising one or more treatment options identified for the individual based at least in part on detection of the fusion nucleic acid molecule or the fusion polypeptide encoded by the fusion nucleic acid molecule, wherein the one or more treatment options comprise an anti-cancer therapy.

In another aspect, provided herein is a method of identifying one or more treatment options for an individual having a cancer, the method comprising: acquiring knowledge of a fusion nucleic acid molecule, or a fusion polypeptide encoded by the fusion nucleic acid molecule, in a sample from the individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; and generating a report comprising one or more treatment options identified for the individual based at least in part on said knowledge, wherein the one or more treatment options comprise an anti-cancer therapy.

In another aspect, provided herein is a method of selecting a treatment for an individual having cancer, the method comprising: acquiring knowledge of a fusion nucleic acid molecule, or a fusion polypeptide encoded by the fusion nucleic acid molecule, in a sample from the individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

In another aspect, provided herein is a method of predicting survival of an individual having a cancer, the method comprising: acquiring knowledge of a fusion nucleic acid molecule, or a fusion polypeptide encoded by the fusion nucleic acid molecule, in a sample from the individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; wherein responsive to the acquisition of said knowledge: the individual is predicted to have longer survival when treated with a treatment comprising an anti-cancer therapy, as compared to survival of an individual whose cancer does not comprise the fusion nucleic acid molecule or the fusion polypeptide encoded by the fusion nucleic acid molecule.

In another aspect, provided herein is a method of predicting survival of an individual having a cancer treated with a treatment comprising an anti-cancer therapy, the method comprising: acquiring knowledge of a fusion nucleic acid molecule, or a fusion polypeptide encoded by the fusion nucleic acid molecule, in a sample from the individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; and wherein responsive to the acquisition of said knowledge: the individual is predicted to have longer survival when treated with a treatment comprising an anti-cancer therapy, as compared to survival of an individual whose cancer does not exhibit the fusion nucleic acid molecule or the fusion polypeptide encoded by the fusion nucleic acid molecule.

In another aspect, provided herein is a method of treating or delaying progression of cancer, the method comprising: acquiring knowledge of a fusion nucleic acid molecule, or a fusion polypeptide encoded by the fusion nucleic acid molecule, in a sample from an individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; and responsive to said knowledge, administering to the individual an effective amount of a treatment that comprises an anti-cancer therapy.

In another aspect, provided herein is a method of treating or delaying progression of cancer, comprising administering to an individual having a cancer an effective amount of a treatment that comprises an anti-cancer therapy, wherein the treatment is administered responsive to acquiring knowledge of a fusion nucleic acid molecule, or a fusion polypeptide encoded by the fusion nucleic acid molecule, in a sample from the individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a method of monitoring, evaluating or screening an individual having a cancer, comprising: acquiring knowledge of a fusion nucleic acid molecule, or a fusion polypeptide encoded by the fusion nucleic acid molecule, in a sample from the individual; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; wherein responsive to the acquisition of said knowledge, the individual is predicted to have an improved response to treatment with an anti-cancer therapy, as compared to an individual whose cancer does not comprise the fusion nucleic acid molecule or the fusion polypeptide encoded by the fusion nucleic acid molecule.

In another aspect, provided herein is a method of assessing a fusion nucleic acid molecule, or a fusion polypeptide encoded by the fusion nucleic acid molecule, in a cancer in an individual, comprising: detecting in a sample from the individual a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; and providing an assessment of the fusion nucleic acid molecule or the fusion polypeptide encoded by the fusion nucleic acid molecule.

In another aspect, provided herein is a method of detecting a fusion nucleic acid molecule, or a fusion polypeptide encoded by the fusion nucleic acid molecule, comprising: detecting in a sample from an individual having a cancer a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a method of detecting the presence or absence of a cancer in an individual, the method comprising: detecting the presence or absence of a cancer in a sample from the individual; and detecting, in a sample from the individual, the presence or absence of a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule. In some embodiments, the method comprises detecting the presence of the cancer in the sample. In some embodiments, the method comprises detecting the presence of the fusion nucleic acid molecule or the fusion polypeptide encoded by the fusion nucleic acid molecule in the sample from the individual.

In another aspect, provided herein is a method of monitoring progression or recurrence of a cancer in an individual, the method comprising: detecting, in a first sample obtained from the individual at a first time point, the presence or absence of a fusion nucleic acid molecule, or a fusion polypeptide encoded by the fusion nucleic acid molecule; detecting, in a second sample obtained from the individual at a second time point after the first time point, the presence or absence of a fusion nucleic acid molecule, or a fusion polypeptide encoded by the fusion nucleic acid molecule; and providing an assessment of cancer progression or cancer recurrence in the individual based, at least in part, on the presence or absence of the fusion nucleic acid molecule or the fusion polypeptide encoded by the fusion nucleic acid molecule in the first sample and/or in the second sample; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule. In some embodiments, the presence of the fusion nucleic acid molecule or the fusion polypeptide encoded by the fusion nucleic acid molecule in the first sample and/or in the second sample identifies the individual as having decreased risk of cancer progression or cancer recurrence when treated with a treatment comprising an anti-cancer therapy. In some embodiments, the method further comprises selecting a treatment, administering a treatment, adjusting a treatment, adjusting a dose of a treatment, or applying a treatment to the individual based, at least in part, on detecting the presence of the fusion nucleic acid molecule or the fusion polypeptide encoded by the fusion nucleic acid molecule in the first sample and/or in the second sample, wherein the treatment comprises an anti-cancer therapy.

In another aspect, provided herein is a method of detecting a fusion nucleic acid molecule, the method comprising: providing a plurality of nucleic acid molecules obtained from a sample from an individual having a cancer, wherein the plurality of nucleic acid molecules comprises nucleic acid molecules corresponding to a fusion nucleic acid molecule, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; optionally, ligating one or more adapters onto one or more nucleic acid molecules from the plurality of nucleic acid molecules; optionally, amplifying the one or more ligated nucleic acid molecules from the plurality of nucleic acid molecules; optionally, capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequence reads correspond to the fusion nucleic acid molecule; analyzing the plurality of sequence reads; and based on the analysis, detecting the presence or absence of the fusion nucleic acid molecule in the sample. In some embodiments, the method further comprises receiving, at one or more processors, sequence read data for the plurality of sequence reads. In some embodiments, analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

In another aspect, provided herein is a method of detecting a fusion nucleic acid molecule, the method comprising: providing a sample from an individual having a cancer, wherein the sample comprises a plurality of nucleic acid molecules; preparing a nucleic acid sequencing library from the plurality of nucleic acid molecules in the sample; amplifying said library; selectively enriching for one or more nucleic acid molecules in said library that comprise nucleotide sequences corresponding to a fusion nucleic acid molecule to produce an enriched sample, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; sequencing the enriched sample, thereby producing a plurality of sequence reads; analyzing the plurality of sequence reads for the presence of the fusion nucleic acid molecule; and detecting, based on the analyzing step, the presence or absence of the fusion nucleic acid molecule in the sample from the individual.

In some embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor, cell-free DNA (cfDNA) fraction or non-tumor blood cell fraction of the liquid biopsy sample. In some embodiments, the one or more adapters comprise amplification primers, flow cell adaptor sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a genomic profile for the individual, based, at least in part, on detecting the presence or absence of the fusion nucleic acid molecule. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting a treatment, administering a treatment, or applying a treatment to the individual based on the generated genomic profile, wherein the treatment comprises an anti-cancer therapy. In some embodiments, the method further comprises generating a report indicating the presence or absence of the fusion nucleic acid molecule in the sample. In some embodiments, the method further comprises generating, by the one or more processors, a report indicating the presence or absence of the fusion nucleic acid molecule in the sample. In some embodiments, the method further comprises transmitting the report to a healthcare provider. In some embodiments, the report is transmitted via a computer network or a peer-to-peer connection.

In another aspect, provided herein is a method of identifying a candidate treatment for a cancer in an individual in need thereof, comprising performing DNA sequencing on a sample obtained from the individual to determine a sequencing mutation profile on a fusion nucleic acid molecule, wherein the sequencing mutation profile identifies the presence or absence of a fusion nucleic acid molecule, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; wherein the candidate treatment comprises an anti-cancer therapy. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the fusion nucleic acid molecule comprising a breakpoint. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the fusion nucleic acid molecule comprising a breakpoint.

In another aspect, provided herein is a method of treating or delaying progression of cancer, comprising: detecting in a sample from an individual having a cancer a fusion nucleic acid molecule, or a fusion polypeptide encoded by the fusion nucleic acid molecule; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule; and administering to the individual an effective amount of a treatment that comprises an anti-cancer therapy.

In some embodiments, the anti-cancer therapy comprises an ALK-targeted therapy. In some embodiments, the ALK-targeted therapy comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, the anti-cancer therapy comprises a kinase inhibitor. In some embodiments, the kinase inhibitor is a multi-kinase inhibitor or an ALK-specific inhibitor. In some embodiments, the kinase inhibitor is a tyrosine kinase inhibitor. In some embodiments, the kinase inhibitor inhibits kinase activity of an ALK polypeptide, e.g., an ALK fusion polypeptide described herein (including without limitation an ALK fusion polypeptide encoded by an ALK fusion nucleic acid listed in Table 1). In some embodiments, the kinase inhibitor is one or more of crizotinib, alectinib, ceritinib, lorlatinib, brigatinib, ensartinib (X-396), repotrectinib (TPX-0005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the anti-cancer therapy comprises a cellular therapy, and wherein the cellular therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the anti-cancer therapy comprises a nucleic acid that inhibits the expression of the fusion nucleic acid molecule or the fusion polypeptide encoded by the fusion nucleic acid molecule. In some embodiments, the anti-cancer therapy comprises a nucleic acid that comprises a double-stranded RNA (dsRNA), a small interfering RNA (siRNA), or a small hairpin RNA (shRNA).

In some embodiments, the fusion nucleic acid molecule is an NRP2-ALK fusion nucleic acid molecule. In some embodiments, the fusion nucleic acid molecule comprises a 5′ breakpoint between exons 8 and 9 of NRP2. In some embodiments, the fusion nucleic acid molecule comprises exons 1-8 of NRP2. In some embodiments, the fusion nucleic acid molecule comprises a 3′ breakpoint between exons 18 and 19 of ALK. In some embodiments, the fusion nucleic acid molecule comprises exons 19-29 of ALK. In some embodiments, the cancer is a uterus leiomyosarcoma or soft tissue inflammatory myofibroblastic tumor.

In some embodiments, the fusion nucleic acid molecule is a PDE3A-ALK fusion nucleic acid molecule. In some embodiments, the fusion nucleic acid molecule comprises a 5′ breakpoint between exons 10 and 11 of PDE3A. In some embodiments, the fusion nucleic acid molecule comprises exons 1-10 of PDE3A. In some embodiments, the fusion nucleic acid molecule comprises a 3′ breakpoint between exons 7 and 8 of ALK. In some embodiments, the fusion nucleic acid molecule comprises exons 8-29 of ALK. In some embodiments, the cancer is a bone osteosarcoma.

In some embodiments, the fusion nucleic acid molecule is a PSMD14-ALK fusion nucleic acid molecule. In some embodiments, the fusion nucleic acid molecule comprises a 5′ breakpoint between exons 1 and 2 of PSMD14. In some embodiments, the fusion nucleic acid molecule comprises exon 1 of PSMD14. In some embodiments, the fusion nucleic acid molecule comprises a 3′ breakpoint between exons 3 and 4 of ALK. In some embodiments, the fusion nucleic acid molecule comprises exons 4-29 of ALK. In some embodiments, the cancer is a bone osteosarcoma.

In some embodiments, the fusion nucleic acid molecule is an SFT2D1-ALK fusion nucleic acid molecule. In some embodiments, the fusion nucleic acid molecule comprises a 5′ breakpoint between exons 1 and 2 of SFT2D1. In some embodiments, the fusion nucleic acid molecule comprises exon 1 of SFT2D1. In some embodiments, the fusion nucleic acid molecule comprises a 3′ breakpoint between exons 5 and 6 of ALK. In some embodiments, the fusion nucleic acid molecule comprises exons 6-29 of ALK. In some embodiments, the cancer is a uterus leiomyosarcoma.

In some embodiments, the fusion nucleic acid molecule is an SLC37A3-ALK fusion nucleic acid molecule. In some embodiments, the fusion nucleic acid molecule comprises a 5′ breakpoint between exons 3 and 4 of SLC37A3. In some embodiments, the fusion nucleic acid molecule comprises exons 1-3 of SLC37A3. In some embodiments, the fusion nucleic acid molecule comprises a 3′ breakpoint between exons 3 and 4 of ALK. In some embodiments, the fusion nucleic acid molecule comprises exons 4-29 of ALK. In some embodiments, the cancer is a soft tissue leiomyosarcoma.

In some embodiments, the fusion nucleic acid molecule is a TANGO6-ALK fusion nucleic acid molecule. In some embodiments, the fusion nucleic acid molecule comprises a 5′ breakpoint between exons 1 and 2 of TANGO6. In some embodiments, the fusion nucleic acid molecule comprises exon 1 of TANGO6. In some embodiments, the fusion nucleic acid molecule comprises a 3′ breakpoint between exons 1 and 2 of ALK. In some embodiments, the fusion nucleic acid molecule comprises exons 2-29 of ALK. In some embodiments, the cancer is a soft tissue undifferentiated cancer/tumor.

In some embodiments, the fusion nucleic acid molecule is a WDR92-ALK fusion nucleic acid molecule. In some embodiments, the fusion nucleic acid molecule comprises a 5′ breakpoint between exons 7 and 8 of WDR92. In some embodiments, the fusion nucleic acid molecule comprises exons 1-7 of WDR92. In some embodiments, the fusion nucleic acid molecule comprises a 3′ breakpoint between exons 1 and 2 of ALK. In some embodiments, the fusion nucleic acid molecule comprises exons 2-29 of ALK. In some embodiments, the cancer is a soft tissue leiomyosarcoma.

In some embodiments, the fusion nucleic acid molecule encodes a fusion polypeptide having ALK kinase activity.

In some embodiments, the fusion nucleic acid molecule is a fusion nucleic acid molecule listed in Table 1, e.g., an ALK fusion molecule listed in Table 1. In some embodiments, the fusion nucleic acid molecule comprises a 5′ and/or 3′ breakpoint listed in Table 1.

In another aspect, provided herein is a kit or article of manufacture comprising a probe or bait for detecting a fusion nucleic acid molecule, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a nucleic acid molecule comprising a fusion nucleic acid molecule, or a fragment thereof comprising a breakpoint or fusion junction, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a vector comprising the nucleic acid molecule according to any one of the above embodiments. In another aspect, provided herein is a host cell comprising the vector according to any one of the above embodiments.

In another aspect, provided herein is an antibody or antibody fragment that specifically binds to a fusion polypeptide, or to a portion thereof, encoded by a fusion nucleic acid molecule, or a fragment thereof comprising a breakpoint or fusion junction, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a kit or article of manufacture comprising an antibody or antibody fragment for detecting a fusion polypeptide, or to a portion thereof, encoded by a fusion nucleic acid molecule, or a fragment thereof comprising a breakpoint or fusion junction, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is the in vitro use of one or more oligonucleotides for detecting a fusion nucleic acid molecule, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a kit or article of manufacture comprising one or more oligonucleotides for detecting a fusion nucleic acid molecule, wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a system, comprising: a memory configured to store one or more program instructions; and one or more processors configured to execute the one or more program instructions, the one or more program instructions when executed by the one or more processors are configured to perform the method according to any one of the embodiments disclosed herein. In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a fusion nucleic acid molecule; and (c) detect, based on the analyzing, the fusion nucleic acid molecule in the sample; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule. In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a fusion nucleic acid molecule; and (c) detect, based on the analyzing, the fusion nucleic acid molecule in the sample; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a non-transitory computer readable storage medium comprising one or more programs executable by one or more computer processors for performing a method according to any one of the embodiments disclosed herein. In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of a fusion nucleic acid molecule; and (c) detecting, using the one or more processors and based on the analyzing, the fusion nucleic acid molecule in the sample; wherein the fusion nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In some embodiments, the sample is from an individual having a cancer. In some embodiments, the cancer is a sarcoma. In some embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the method further comprises generating, based at least in part on the detecting, a genomic profile for the sample. In some embodiments, the individual is administered a treatment based at least in part on the genomic profile. In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test.

In another aspect, provided herein is an anti-cancer therapy for use in the method according to any one of the above embodiments. In another aspect, provided herein is an anti-cancer therapy for use in a method of treating or delaying progression of cancer, wherein the method comprises administering the anti-cancer therapy to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule. In another aspect, provided herein is an anti-cancer therapy for use in the manufacture of a medicament for treating or delaying progression of cancer, wherein the medicament is to be administered to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is: (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule.

In another aspect, provided herein is a method of identifying an individual having a cancer who may benefit from a treatment comprising an anti-cancer therapy, the method comprising detecting in a sample from the individual a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein detection of the fusion nucleic acid molecule or the fusion polypeptide encoded by the fusion nucleic acid molecule in the sample identifies the individual as one who may benefit from the treatment comprising the anti-cancer therapy.

In another aspect, provided herein is a method of selecting a treatment for an individual having a cancer, the method comprising detecting in a sample from the individual a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein detection of the fusion nucleic acid molecule or the fusion polypeptide encoded by the fusion nucleic acid molecule in the sample identifies the individual as one who may benefit from the treatment comprising an anti-cancer therapy.

In another aspect, provided herein is a method of identifying one or more treatment options for an individual having a cancer, the method comprising: detecting in a sample from the individual a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; generating a report comprising one or more treatment options identified for the individual based at least in part on detection of the fusion nucleic acid molecule or the fusion polypeptide encoded by the fusion nucleic acid molecule, wherein the one or more treatment options comprise an anti-cancer therapy.

In another aspect, provided herein is a method of identifying one or more treatment options for an individual having a cancer, the method comprising: acquiring knowledge of a fusion nucleic acid molecule, or a fusion polypeptide encoded by the fusion nucleic acid molecule, in a sample from the individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; generating a report comprising one or more treatment options identified for the individual based at least in part on said knowledge, wherein the one or more treatment options comprise an anti-cancer therapy.

In another aspect, provided herein is a method of selecting a treatment for an individual having cancer, the method comprising: acquiring knowledge of a fusion nucleic acid molecule, or a fusion polypeptide encoded by the fusion nucleic acid molecule, in a sample from the individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

In another aspect, provided herein is a method of predicting survival of an individual having a cancer, the method comprising: acquiring knowledge of a fusion nucleic acid molecule, or a fusion polypeptide encoded by the fusion nucleic acid molecule, in a sample from the individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein responsive to the acquisition of said knowledge: the individual is predicted to have longer survival when treated with a treatment comprising an anti-cancer therapy, as compared to survival of an individual whose cancer does not comprise the fusion nucleic acid molecule or the fusion polypeptide encoded by the fusion nucleic acid molecule.

In another aspect, provided herein is a method of predicting survival of an individual having a cancer treated with a treatment comprising an anti-cancer therapy, the method comprising: acquiring knowledge of a fusion nucleic acid molecule, or a fusion polypeptide encoded by the fusion nucleic acid molecule, in a sample from the individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein responsive to the acquisition of said knowledge: the individual is predicted to have longer survival when treated with a treatment comprising an anti-cancer therapy, as compared to survival of an individual whose cancer does not exhibit the fusion nucleic acid molecule or the fusion polypeptide encoded by the fusion nucleic acid molecule.

In another aspect, provided herein is a method of treating or delaying progression of cancer, the method comprising: acquiring knowledge of a fusion nucleic acid molecule, or a fusion polypeptide encoded by the fusion nucleic acid molecule, in a sample from an individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; and responsive to said knowledge, administering to the individual an effective amount of a treatment that comprises an anti-cancer therapy.

In another aspect, provided herein is a method of treating or delaying progression of cancer, comprising administering to an individual having a cancer an effective amount of a treatment that comprises an anti-cancer therapy, wherein the treatment is administered responsive to acquiring knowledge of a fusion nucleic acid molecule, or a fusion polypeptide encoded by the fusion nucleic acid molecule, in a sample from the individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a method of monitoring, evaluating or screening an individual having a cancer, comprising: acquiring knowledge of a fusion nucleic acid molecule, or a fusion polypeptide encoded by the fusion nucleic acid molecule, in a sample from the individual; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein responsive to the acquisition of said knowledge, the individual is predicted to have an improved response to treatment with an anti-cancer therapy, as compared to an individual whose cancer does not comprise the fusion nucleic acid molecule or the fusion polypeptide encoded by the fusion nucleic acid molecule.

In another aspect, provided herein is a method of assessing a fusion nucleic acid molecule, or a fusion polypeptide encoded by the fusion nucleic acid molecule, in a cancer in an individual, comprising: detecting in a sample from the individual a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; and providing an assessment of the fusion nucleic acid molecule or the fusion polypeptide encoded by the fusion nucleic acid molecule.

In another aspect, provided herein is a method of detecting a fusion nucleic acid molecule, or a fusion polypeptide encoded by the fusion nucleic acid molecule, comprising: detecting in a sample from an individual having a cancer a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a method of detecting the presence or absence of a cancer in an individual, the method comprising: detecting the presence or absence of a cancer in a sample from the individual; and detecting, in a sample from the individual, the presence or absence of a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule. In some embodiments, the method comprises detecting the presence of the cancer in the sample. In some embodiments, the method comprises detecting the presence of the fusion nucleic acid molecule or the fusion polypeptide encoded by the fusion nucleic acid molecule in the sample from the individual.

In another aspect, provided herein is a method of monitoring progression or recurrence of a cancer in an individual, the method comprising: detecting, in a first sample obtained from the individual at a first time point, the presence or absence of a fusion nucleic acid molecule, or a fusion polypeptide encoded by the fusion nucleic acid molecule; detecting, in a second sample obtained from the individual at a second time point after the first time point, the presence or absence of a fusion nucleic acid molecule, or a fusion polypeptide encoded by the fusion nucleic acid molecule; and providing an assessment of cancer progression or cancer recurrence in the individual based, at least in part, on the presence or absence of the fusion nucleic acid molecule or the fusion polypeptide encoded by the fusion nucleic acid molecule in the first sample and/or in the second sample; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule. In some embodiments, the presence of the fusion nucleic acid molecule or the fusion polypeptide encoded by the fusion nucleic acid molecule in the first sample and/or in the second sample identifies the individual as having decreased risk of cancer progression or cancer recurrence when treated with a treatment comprising an anti-cancer therapy. In some embodiments, the method further comprises selecting a treatment, administering a treatment, adjusting a treatment, adjusting a dose of a treatment, or applying a treatment to the individual based, at least in part, on detecting the presence of the fusion nucleic acid molecule or the fusion polypeptide encoded by the fusion nucleic acid molecule in the first sample and/or in the second sample, wherein the treatment comprises an anti-cancer therapy.

In another aspect, provided herein is a method of detecting a fusion nucleic acid molecule, the method comprising: providing a plurality of nucleic acid molecules obtained from a sample from an individual having a cancer, wherein the plurality of nucleic acid molecules comprises nucleic acid molecules corresponding to a fusion nucleic acid molecule, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; optionally, ligating one or more adapters onto one or more nucleic acid molecules from the plurality of nucleic acid molecules; optionally, amplifying the one or more ligated nucleic acid molecules from the plurality of nucleic acid molecules; optionally, capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequence reads correspond to the fusion nucleic acid molecule; analyzing the plurality of sequence reads; and based on the analysis, detecting the presence or absence of the fusion nucleic acid molecule in the sample. In some embodiments, the method further comprises receiving, at one or more processors, sequence read data for the plurality of sequence reads. In some embodiments, analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

In another aspect, provided herein is a method of detecting a fusion nucleic acid molecule, the method comprising: providing a sample from an individual having a cancer, wherein the sample comprises a plurality of nucleic acid molecules; preparing a nucleic acid sequencing library from the plurality of nucleic acid molecules in the sample; amplifying said library; selectively enriching for one or more nucleic acid molecules in said library that comprise nucleotide sequences corresponding to a fusion nucleic acid molecule to produce an enriched sample, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; sequencing the enriched sample, thereby producing a plurality of sequence reads; analyzing the plurality of sequence reads for the presence of the fusion nucleic acid molecule; and detecting, based on the analyzing step, the presence or absence of the fusion nucleic acid molecule in the sample from the individual.

In some embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor, cell-free DNA (cfDNA) fraction or non-tumor blood cell fraction of the liquid biopsy sample. In some embodiments, the one or more adapters comprise amplification primers, flow cell adaptor sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a genomic profile for the individual, based, at least in part, on detecting the presence or absence of the fusion nucleic acid molecule. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting a treatment, administering a treatment, or applying a treatment to the individual based on the generated genomic profile, wherein the treatment comprises an anti-cancer therapy. In some embodiments, the method further comprises generating a report indicating the presence or absence of the fusion nucleic acid molecule in the sample. In some embodiments, the method further comprises generating, by the one or more processors, a report indicating the presence or absence of the fusion nucleic acid molecule in the sample. In some embodiments, the method further comprises transmitting the report to a healthcare provider. In some embodiments, the report is transmitted via a computer network or a peer-to-peer connection.

In another aspect, provided herein is a method of identifying a candidate treatment for a cancer in an individual in need thereof, comprising performing DNA sequencing on a sample obtained from the individual to determine a sequencing mutation profile on a fusion nucleic acid molecule, wherein the sequencing mutation profile identifies the presence or absence of a fusion nucleic acid molecule, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; wherein the candidate treatment comprises an anti-cancer therapy.

In another aspect, provided herein is a method of treating or delaying progression of cancer, comprising: detecting in a sample from an individual having a cancer a fusion nucleic acid molecule, or a fusion polypeptide encoded by the fusion nucleic acid molecule; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule; and administering to the individual an effective amount of a treatment that comprises an anti-cancer therapy.

In some embodiments, the anti-cancer therapy comprises an NTRK1-targeted therapy and/or an NTRK3-targeted therapy. In some embodiments, the anti-cancer therapy comprises a kinase inhibitor. In some embodiments, the kinase inhibitor is a multi-kinase inhibitor, an NTRK1-specific inhibitor, or an NTRK3-specific inhibitor. In some embodiments, the kinase inhibitor is a tyrosine kinase inhibitor. In some embodiments, the kinase inhibitor inhibits kinase activity of an NTRK1 or NTRK3 polypeptide, e.g., an NTRK1 or NTRK3 fusion polypeptide described herein (including without limitation an NTRK1 or NTRK3 fusion polypeptide encoded by an NTRK1 or NTRK3 fusion nucleic acid listed in Table 1). In some embodiments, the kinase inhibitor is one or more of AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), LOXO-195, a macrocyclic compound, ONO-5390556, oxindole 3, pegcantratinib (SNA-120), PHA-848125, PLX7486, a pyrazole derivative, a pyrazolo[1, 5a]pyrimidine, a pyridocarbazole, a pyridoquinazolinyl, a pyridotriazole, a pyrrolidinyl thiourea, a pyrrolidinyl urea, a pyrrolo[2, 3-d]pyrimidine, a quinazolinyl, repotrectinib, Ro 08-2750, a substituted pyrazolo[1,5a]pyrimidine, sitravatinib, SNA-125, tavilermide, thiazole 20h, ARRY-772, AZD7451, belizatinib, selitrectinib, crizotinib, ONO-7579, merestinib, ensartinib, TSR-011, MGCD516, altiratinib, cabozantinib, XL-184, DCC-2701, F17752, regorafenib, dovitinib, BMS-754807, ENMD-2076, BMS-777607, midostaurin, MK5108, PF-03814735, SNS-314, nintedanib, ponatinib, foretinib, AZD 1480, or VMD-928. In some embodiments, the kinase inhibitor is ARRY-470 or larotrectinib, AZ-23, danusertib (PHA-739358), entrectinib, lestaurtinib (CEP-701), AZD7451, belizatinib, selitrectinib, or crizotinib. In some embodiments, the anti-cancer therapy comprises a cellular therapy, and wherein the cellular therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the anti-cancer therapy comprises a nucleic acid that inhibits the expression of the fusion nucleic acid molecule or the fusion polypeptide encoded by the fusion nucleic acid molecule. In some embodiments, the anti-cancer therapy comprises a nucleic acid that comprises a double-stranded RNA (dsRNA), a small interfering RNA (siRNA), or a small hairpin RNA (shRNA).

In some embodiments, the fusion nucleic acid molecule is a GPA33-NTRK1 fusion nucleic acid molecule. In some embodiments, the fusion nucleic acid molecule comprises a 5′ breakpoint between exons 4 and 5 of GPA33. In some embodiments, the fusion nucleic acid molecule comprises exons 1-4 of GPA33. In some embodiments, the fusion nucleic acid molecule comprises a 3′ breakpoint between exons 4 and 5 of NTRK1. In some embodiments, the fusion nucleic acid molecule comprises exons 5-17 of NTRK1. In some embodiments, the cancer is a soft tissue malignant peripheral nerve sheath tumor (MPNST).

In some embodiments, the fusion nucleic acid molecule is a FAM19A2-NTRK1 fusion nucleic acid molecule. In some embodiments, the fusion nucleic acid molecule comprises a 5′ breakpoint between exons 1 and 2 of FAM19A2. In some embodiments, the fusion nucleic acid molecule comprises exon 1 of FAM19A2. In some embodiments, the fusion nucleic acid molecule comprises a 3′ breakpoint between exons 3 and 4 of NTRK1. In some embodiments, the fusion nucleic acid molecule comprises exons 4-17 of NTRK1. In some embodiments, the cancer is a soft tissue sarcoma not otherwise specified (nos).

In some embodiments, the fusion nucleic acid molecule is a CPSF6-NTRK1 fusion nucleic acid molecule. In some embodiments, the fusion nucleic acid molecule comprises a 5′ breakpoint between exons 7 and 8 of CPSF6. In some embodiments, the fusion nucleic acid molecule comprises exons 1-7 of CPSF6. In some embodiments, the fusion nucleic acid molecule comprises a 3′ breakpoint between exons 11 and 12 of NTRK1. In some embodiments, the fusion nucleic acid molecule comprises exons 12-17 of NTRK1. In some embodiments, the cancer is a soft tissue liposarcoma.

In some embodiments, the fusion nucleic acid molecule is a SUCO-NTRK1 fusion nucleic acid molecule. In some embodiments, the fusion nucleic acid molecule comprises a 5′ breakpoint between exons 10 and 11 of SUCO. In some embodiments, the fusion nucleic acid molecule comprises exons 1-10 of SUCO. In some embodiments, the fusion nucleic acid molecule comprises a 3′ breakpoint between exons 2 and 3 of NTRK1. In some embodiments, the fusion nucleic acid molecule comprises exons 3-17 of NTRK1. In some embodiments, the cancer is a soft tissue liposarcoma.

In some embodiments, the fusion nucleic acid molecule is a CACYBP-NTRK1 fusion nucleic acid molecule. In some embodiments, the fusion nucleic acid molecule comprises a 5′ breakpoint between exons 2 and 3 of CACYBP. In some embodiments, the fusion nucleic acid molecule comprises exons 1 and 2 of CACYBP. In some embodiments, the fusion nucleic acid molecule comprises a 3′ breakpoint between exons 8 and 9 of NTRK1. In some embodiments, the fusion nucleic acid molecule comprises exons 9-17 of NTRK1. In some embodiments, the cancer is a uterus sarcoma.

In some embodiments, the fusion nucleic acid molecule is a ZNF382-NTRK1 fusion nucleic acid molecule. In some embodiments, the fusion nucleic acid molecule comprises a 5′ breakpoint between exons 4 and 5 of ZNF382. In some embodiments, the fusion nucleic acid molecule comprises exons 1-4 of ZNF382. In some embodiments, the fusion nucleic acid molecule comprises a 3′ breakpoint between exons 8 and 9 of NTRK1. In some embodiments, the fusion nucleic acid molecule comprises exons 9-17 of NTRK1. In some embodiments, the cancer is a soft tissue leiomyosarcoma.

In some embodiments, the fusion nucleic acid molecule is a fusion nucleic acid molecule listed in Table 1, e.g., an NTRK1 or NTRK3 fusion molecule listed in Table 1. In some embodiments, the fusion nucleic acid molecule comprises a 5′ and/or 3′ breakpoint listed in Table 1.

In some embodiments, the fusion nucleic acid molecule encodes a fusion polypeptide having NTRK1 kinase activity.

In some embodiments, the fusion nucleic acid molecule is an NDE1-NTRK3 fusion nucleic acid molecule. In some embodiments, the fusion nucleic acid molecule comprises a 5′ breakpoint between exons 6 and 7 of NDE1. In some embodiments, the fusion nucleic acid molecule comprises exons 1-6 of NDE1. In some embodiments, the fusion nucleic acid molecule comprises a 3′ breakpoint between exons 13 and 14 of NTRK3. In some embodiments, the fusion nucleic acid molecule comprises exons 14-19 of NTRK3. In some embodiments, the cancer is a soft tissue myxofibrosarcoma.

In some embodiments, the fusion nucleic acid molecule is a DGCR5-NTRK3 fusion nucleic acid molecule. In some embodiments, the fusion nucleic acid molecule comprises a 5′ breakpoint between exons 1 and 2 of DGCR5. In some embodiments, the fusion nucleic acid molecule comprises exon 1 of DGCR5. In some embodiments, the fusion nucleic acid molecule comprises a 3′ breakpoint between exons 2 and 3 of NTRK3. In some embodiments, the fusion nucleic acid molecule comprises exons 3-19 of NTRK3. In some embodiments, the cancer is a soft tissue leiomyosarcoma.

In some embodiments, the fusion nucleic acid molecule is a UBE2Q2P1-NTRK3 fusion nucleic acid molecule. In some embodiments, the fusion nucleic acid molecule comprises a 5′ breakpoint between exons 5 and 6 of UBE2Q2P1. In some embodiments, the fusion nucleic acid molecule comprises exons 1-5 of UBE2Q2P1. In some embodiments, the fusion nucleic acid molecule comprises a 3′ breakpoint between exons 5 and 6 of NTRK3. In some embodiments, the fusion nucleic acid molecule comprises exons 6-19 of NTRK3. In some embodiments, the cancer is a soft tissue sarcoma (nos).

In some embodiments, the fusion nucleic acid molecule encodes a fusion polypeptide having NTRK3 kinase activity.

In some embodiments according to any of the embodiments described herein, the anti-cancer therapy or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments according to any of the embodiments described herein, the cancer is a sarcoma. In some embodiments, the cancer is a uterus leiomyosarcoma, soft tissue inflammatory myofibroblastic tumor, soft tissue sarcoma not otherwise specified (nos), bone osteosarcoma, soft tissue leiomyosarcoma, soft tissue sarcoma undifferentiated, soft tissue malignant peripheral nerve sheath tumor (mpnst), soft tissue liposarcoma, uterus sarcoma not otherwise specified (nos), or soft tissue myxofibrosarcoma.

In some embodiments according to any of the embodiments described herein, the method further comprises obtaining the sample from the individual. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the method further comprises acquiring knowledge of or detecting the fusion nucleic acid molecule or the fusion polypeptide encoded by the fusion nucleic acid molecule in a tissue biopsy sample, in a liquid biopsy sample, or in both a tissue biopsy sample and a liquid biopsy sample, from the individual. In some embodiments, the acquiring knowledge comprises detecting the fusion nucleic acid molecule or the fusion polypeptide encoded by the fusion nucleic acid molecule in the sample. In some embodiments, the fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS). In some embodiments, detecting the fusion polypeptide comprises detecting a portion of the fusion polypeptide that is encoded by a fragment of the fusion nucleic acid molecule that comprises a breakpoint or a fusion junction. In some embodiments, the fusion polypeptide is detected in the sample by one or more of: immunoblotting, enzyme linked immunosorbent assay (ELISA), immunohistochemistry, or mass spectrometry. In some embodiments, the method further comprises selectively enriching for one or more nucleic acids in the sample comprising nucleotide sequences corresponding to the fusion nucleic acid molecule; wherein the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the fusion nucleic acid molecule and producing nucleic acid hybrids; and (b) isolating the nucleic acid hybrids to produce the enriched sample. In some embodiments, the one or more bait molecules comprise a capture nucleic acid molecule configured to hybridize to a nucleotide sequence corresponding to the fusion nucleic acid molecule. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, the selectively enriching comprises amplifying the one or more nucleic acids comprising nucleotide sequences corresponding to the fusion nucleic acid molecule using a polymerase chain reaction (PCR) to produce an enriched sample. In some embodiments, the method further comprises sequencing the enriched sample.

In some embodiments according to any of the embodiments described herein, the individual is a human.

In another aspect, provided herein is a kit or article of manufacture comprising a probe or bait for detecting a fusion nucleic acid molecule, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a nucleic acid molecule comprising a fusion nucleic acid molecule, or a fragment thereof comprising a breakpoint or fusion junction, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a vector comprising the nucleic acid molecule according to any one of the above embodiments. In another aspect, provided herein is a host cell comprising the vector according to any one of the above embodiments.

In another aspect, provided herein is an antibody or antibody fragment that specifically binds to a fusion polypeptide, or to a portion thereof, encoded by a fusion nucleic acid molecule, or a fragment thereof comprising a breakpoint or fusion junction, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a kit or article of manufacture comprising an antibody or antibody fragment for detecting a fusion polypeptide, or to a portion thereof, encoded by a fusion nucleic acid molecule, or a fragment thereof comprising a breakpoint or fusion junction, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is the in vitro use of one or more oligonucleotides for detecting a fusion nucleic acid molecule, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a kit or article of manufacture comprising one or more oligonucleotides for detecting a fusion nucleic acid molecule, wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a system, comprising: a memory configured to store one or more program instructions; and one or more processors configured to execute the one or more program instructions, the one or more program instructions when executed by the one or more processors are configured to perform the method according to any one of the embodiments disclosed herein. In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a fusion nucleic acid molecule; and (c) detect, based on the analyzing, the fusion nucleic acid molecule in the sample; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule. In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of a fusion nucleic acid molecule; and (c) detect, based on the analyzing, the fusion nucleic acid molecule in the sample; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In another aspect, provided herein is a non-transitory computer readable storage medium comprising one or more programs executable by one or more computer processors for performing a method according to any one of the embodiments disclosed herein. In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of a fusion nucleic acid molecule; and (c) detecting, using the one or more processors and based on the analyzing, the fusion nucleic acid molecule in the sample; wherein the fusion nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

In some embodiments, the sample is from an individual having a cancer. In some embodiments, the cancer is a sarcoma. In some embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS). In some embodiments, the method further comprises generating, based at least in part on the detecting, a genomic profile for the sample. In some embodiments, the individual is administered a treatment based at least in part on the genomic profile. In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test.

In another aspect, provided herein is an anti-cancer therapy for use in the method according to any one of the above embodiments. In another aspect, provided herein is an anti-cancer therapy for use in a method of treating or delaying progression of cancer, wherein the method comprises administering the anti-cancer therapy to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule. In another aspect, provided herein is an anti-cancer therapy for use in the manufacture of a medicament for treating or delaying progression of cancer, wherein the medicament is to be administered to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is: (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows that analyzing DNA and RNA detects most gene fusions and rearrangements in sarcoma. Total cases are indicated by bar label. For the bar corresponding to each sarcoma type, fusions/rearrangements detected in DNA and RNA are shown on bottom, those detected in RNA only are shown in middle, and those detected in DNA only are shown on top.

FIG. 2 shows that diverse gene fusions and rearrangements were seen across a wide range of sarcomas.

FIG. 3 shows that analysis of RNA detects ALK fusions with distinct breakpoints not covered by DNA baiting that covers canonical non-small cell lung cancer (NSCLC) breakpoints.

FIGS. 4A & 4B show that, of 41 NTRK1/3 gene fusions detected in DNA, 88% were confirmed by analyzing RNA (5 in DNA only, 36 in DNA and RNA; FIG. 4A). An additional 39 fusions were detected in RNA only (FIG. 4B); 100% were outside of DNA-baited region (NTRK1 intron 7, 8, and 9; NTRK3 no intron baiting).

FIG. 5 depicts an exemplary device, in accordance with some embodiments.

FIG. 6 depicts an exemplary system, in accordance with some embodiments.

FIG. 7 depicts a block diagram of an exemplary process for detecting a fusion nucleic acid molecule, in accordance with some embodiments.

### DETAILED DESCRIPTION

The present disclosure relates generally to detecting kinase fusions in cancer, as well as methods of treatment, and uses related thereto.

Kinase fusions are an important class of targetable oncogenic driver variants. The present disclosure describes the results of comprehensive genomic profiling of DNA and RNA from more than 9,900 sarcoma tissue specimens. These analyses identified diverse rearrangements leading to fusion genes involving ALK, NTRK1, and NTRK3. Importantly, analysis of RNA through hybrid capture-based sequencing led to the identification of fusion genes that were not detected by hybrid capture of DNA using baits corresponding to canonical non-small cell lung cancer (NSCLC) breakpoints, thereby increasing the sensitivity for atypical fusions with non-canonical breakpoints. Accordingly, without wishing to be bound by theory, it is thought that the presence of a kinase fusion described herein in a sample, e.g., a liquid biopsy sample comprising ctDNA and/or a tissue sample such as a tumor biopsy, from individuals having cancer may identify cancer patients who are likely to respond to treatment with an anti-cancer therapy such as a targeted anti-cancer therapy, e.g., as described herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-0-methyl-, 2-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“κ”) and lambda (“λ”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2, or Megalign (DNASTAR) software. Those skilled in the art can determine appropriate parameters for measuring alignment, including any algorithms needed to achieve maximal alignment over the full-length of the sequences being compared.

The term “detection” includes any means of detecting, including direct and indirect detection. The term “biomarker” as used herein (e.g., a “biomarker” such as a kinase fusion or a fusion nucleic acid molecule or polypeptide described herein) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy including a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., a kinase fusion or a fusion nucleic acid molecule or polypeptide described herein) described herein. In certain embodiments, the manufacture or kit is promoted, distributed, or sold as a unit for performing the methods described herein.

The phrase “based on” when used herein means that the information about one or more biomarkers (e.g., a kinase fusion or a fusion nucleic acid molecule or polypeptide described herein) is used to inform a treatment decision, information provided on a package insert, or marketing/promotional guidance, etc.

The terms “allele frequency” and “allele fraction” are used interchangeably herein and refer to the fraction of sequence reads corresponding to a particular allele relative to the total number of sequence reads for a genomic locus. The terms “variant allele frequency” and “variant allele fraction” are used interchangeably herein and refer to the fraction of sequence reads corresponding to a particular variant allele relative to the total number of sequence reads for a genomic locus.

### III. Methods, Systems, and Devices

Certain aspects of the present disclosure relate to methods of identifying an individual having a cancer who may benefit from a treatment comprising an anti-cancer therapy, the method comprising detecting in a sample from the individual a fusion nucleic acid molecule of the present disclosure, or a fusion polypeptide encoded by the fusion nucleic acid molecule; wherein detection of the fusion nucleic acid molecule or the fusion polypeptide encoded by the fusion nucleic acid molecule in the sample identifies the individual as one who may benefit from the treatment comprising the anti-cancer therapy.

Certain aspects of the present disclosure relate to methods of selecting a treatment for an individual having a cancer, the method comprising detecting in a sample from the individual a fusion nucleic acid molecule of the present disclosure, or a fusion polypeptide encoded by the fusion nucleic acid molecule; wherein detection of the fusion nucleic acid molecule or the fusion polypeptide encoded by the fusion nucleic acid molecule in the sample identifies the individual as one who may benefit from the treatment comprising an anti-cancer therapy.

Certain aspects of the present disclosure relate to methods of identifying one or more treatment options for an individual having a cancer, the method comprising: detecting in a sample from the individual a fusion nucleic acid molecule of the present disclosure, or a fusion polypeptide encoded by the fusion nucleic acid molecule; and generating a report comprising one or more treatment options identified for the individual based at least in part on detection of the fusion nucleic acid molecule or the fusion polypeptide encoded by the fusion nucleic acid molecule, wherein the one or more treatment options comprise an anti-cancer therapy.

Certain aspects of the present disclosure relate to methods of identifying one or more treatment options for an individual having a cancer, the method comprising: acquiring knowledge of a fusion nucleic acid molecule of the present disclosure, or a fusion polypeptide encoded by the fusion nucleic acid molecule, in a sample from the individual; and generating a report comprising one or more treatment options identified for the individual based at least in part on said knowledge, wherein the one or more treatment options comprise an anti-cancer therapy.

Certain aspects of the present disclosure relate to methods of selecting a treatment for an individual having cancer, the method comprising: acquiring knowledge of a fusion nucleic acid molecule of the present disclosure, or a fusion polypeptide encoded by the fusion nucleic acid molecule, in a sample from the individual; wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

Certain aspects of the present disclosure relate to methods of predicting survival of an individual having a cancer, the method comprising: acquiring knowledge of a fusion nucleic acid molecule of the present disclosure, or a fusion polypeptide encoded by the fusion nucleic acid molecule, in a sample from the individual; wherein responsive to the acquisition of said knowledge: the individual is predicted to have longer survival when treated with a treatment comprising an anti-cancer therapy, as compared to survival of an individual whose cancer does not comprise the fusion nucleic acid molecule or the fusion polypeptide encoded by the fusion nucleic acid molecule.

Certain aspects of the present disclosure relate to methods of predicting survival of an individual having a cancer treated with a treatment comprising an anti-cancer therapy, the method comprising: acquiring knowledge of a fusion nucleic acid molecule of the present disclosure, or a fusion polypeptide encoded by the fusion nucleic acid molecule, in a sample from the individual; and wherein responsive to the acquisition of said knowledge: the individual is predicted to have longer survival when treated with a treatment comprising an anti-cancer therapy, as compared to survival of an individual whose cancer does not exhibit the fusion nucleic acid molecule or the fusion polypeptide encoded by the fusion nucleic acid molecule.

Certain aspects of the present disclosure relate to methods of treating or delaying progression of cancer, the method comprising: acquiring knowledge of a fusion nucleic acid molecule of the present disclosure, or a fusion polypeptide encoded by the fusion nucleic acid molecule, in a sample from an individual; and responsive to said knowledge, administering to the individual an effective amount of a treatment that comprises an anti-cancer therapy.

Certain aspects of the present disclosure relate to methods of treating or delaying progression of cancer, comprising administering to an individual having a cancer an effective amount of a treatment that comprises an anti-cancer therapy, wherein the treatment is administered responsive to acquiring knowledge of a fusion nucleic acid molecule of the present disclosure, or a fusion polypeptide encoded by the fusion nucleic acid molecule, in a sample from the individual.

Certain aspects of the present disclosure relate to methods of monitoring, evaluating or screening an individual having a cancer, comprising: acquiring knowledge of a fusion nucleic acid molecule of the present disclosure, or a fusion polypeptide encoded by the fusion nucleic acid molecule, in a sample from the individual; wherein responsive to the acquisition of said knowledge, the individual is predicted to have an improved response to treatment with an anti-cancer therapy, as compared to an individual whose cancer does not comprise the fusion nucleic acid molecule or the fusion polypeptide encoded by the fusion nucleic acid molecule.

Certain aspects of the present disclosure relate to methods of assessing a fusion nucleic acid molecule, or a fusion polypeptide encoded by the fusion nucleic acid molecule, in a cancer in an individual, comprising: detecting in a sample from the individual a fusion nucleic acid molecule of the present disclosure, or a fusion polypeptide encoded by the fusion nucleic acid molecule; and providing an assessment of the fusion nucleic acid molecule or the fusion polypeptide encoded by the fusion nucleic acid molecule.

Certain aspects of the present disclosure relate to methods of detecting a fusion nucleic acid molecule, or a fusion polypeptide encoded by the fusion nucleic acid molecule, comprising: detecting in a sample from an individual having a cancer a fusion nucleic acid molecule of the present disclosure, or a fusion polypeptide encoded by the fusion nucleic acid molecule.

Certain aspects of the present disclosure relate to methods of detecting the presence or absence of a cancer in an individual, the method comprising: detecting the presence or absence of a cancer in a sample from the individual; and detecting, in a sample from the individual, the presence or absence of a fusion nucleic acid molecule of the present disclosure, or a fusion polypeptide encoded by the fusion nucleic acid molecule. In some embodiments, the method comprises detecting the presence of the cancer in the sample. In some embodiments, the method comprises detecting the presence of the fusion nucleic acid molecule or the fusion polypeptide encoded by the fusion nucleic acid molecule in the sample from the individual.

Certain aspects of the present disclosure relate to methods of monitoring progression or recurrence of a cancer in an individual, the method comprising: detecting, in a first sample obtained from the individual at a first time point, the presence or absence of a fusion nucleic acid molecule of the present disclosure, or a fusion polypeptide encoded by the fusion nucleic acid molecule; detecting, in a second sample obtained from the individual at a second time point after the first time point, the presence or absence of a fusion nucleic acid molecule of the present disclosure, or a fusion polypeptide encoded by the fusion nucleic acid molecule; and providing an assessment of cancer progression or cancer recurrence in the individual based, at least in part, on the presence or absence of the fusion nucleic acid molecule or the fusion polypeptide encoded by the fusion nucleic acid molecule in the first sample and/or in the second sample. In some embodiments, the presence of the fusion nucleic acid molecule or the fusion polypeptide encoded by the fusion nucleic acid molecule in the first sample and/or in the second sample identifies the individual as having decreased risk of cancer progression or cancer recurrence when treated with a treatment comprising an anti-cancer therapy. In some embodiments, the method further comprises selecting a treatment, administering a treatment, adjusting a treatment, adjusting a dose of a treatment, or applying a treatment to the individual based, at least in part, on detecting the presence of the fusion nucleic acid molecule or the fusion polypeptide encoded by the fusion nucleic acid molecule in the first sample and/or in the second sample, wherein the treatment comprises an anti-cancer therapy.

Certain aspects of the present disclosure relate to methods of detecting a fusion nucleic acid molecule, the method comprising: providing a plurality of nucleic acid molecules obtained from a sample from an individual having a cancer, wherein the plurality of nucleic acid molecules comprises nucleic acid molecules corresponding to a fusion nucleic acid molecule of the present disclosure; optionally, ligating one or more adapters onto one or more nucleic acid molecules from the plurality of nucleic acid molecules; optionally, amplifying the one or more ligated nucleic acid molecules from the plurality of nucleic acid molecules; optionally, capturing amplified nucleic acid molecules from the amplified nucleic acid molecules; sequencing, by a sequencer, the captured nucleic acid molecules to obtain a plurality of sequence reads that represent the captured nucleic acid molecules, wherein one or more of the plurality of sequence reads correspond to the fusion nucleic acid molecule; analyzing the plurality of sequence reads; and based on the analysis, detecting the presence or absence of the fusion nucleic acid molecule in the sample. In some embodiments, the method further comprises receiving, at one or more processors, sequence read data for the plurality of sequence reads. In some embodiments, analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

Certain aspects of the present disclosure relate to methods of detecting a fusion nucleic acid molecule, the method comprising: providing a sample from an individual having a cancer, wherein the sample comprises a plurality of nucleic acid molecules; preparing a nucleic acid sequencing library from the plurality of nucleic acid molecules in the sample; amplifying said library; selectively enriching for one or more nucleic acid molecules in said library that comprise nucleotide sequences corresponding to a fusion nucleic acid molecule of the present disclosure to produce an enriched sample; sequencing the enriched sample, thereby producing a plurality of sequence reads; analyzing the plurality of sequence reads for the presence of the fusion nucleic acid molecule; and detecting, based on the analyzing step, the presence or absence of the fusion nucleic acid molecule in the sample from the individual. In some embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor, cell-free DNA (cfDNA) fraction or non-tumor blood cell fraction of the liquid biopsy sample. In some embodiments, the one or more adapters comprise amplification primers, flow cell adaptor sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a genomic profile for the individual, based, at least in part, on detecting the presence or absence of the fusion nucleic acid molecule. In some embodiments, the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile for the individual further comprises results from a nucleic acid sequencing-based test. In some embodiments, the method further comprises selecting a treatment, administering a treatment, or applying a treatment to the individual based on the generated genomic profile, wherein the treatment comprises an anti-cancer therapy. In some embodiments, the method further comprises generating a report indicating the presence or absence of the fusion nucleic acid molecule in the sample. In some embodiments, the method further comprises generating, by the one or more processors, a report indicating the presence or absence of the fusion nucleic acid molecule in the sample. In some embodiments, the method further comprises transmitting the report to a healthcare provider. In some embodiments, the report is transmitted via a computer network or a peer-to-peer connection.

Certain aspects of the present disclosure relate to methods of identifying a candidate treatment for a cancer in an individual in need thereof, comprising performing DNA sequencing on a sample obtained from the individual to determine a sequencing mutation profile on a fusion nucleic acid molecule of the present disclosure, wherein the sequencing mutation profile identifies the presence or absence of a fusion nucleic acid molecule; wherein the candidate treatment comprises an anti-cancer therapy. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the fusion nucleic acid molecule comprising a breakpoint. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the fusion nucleic acid molecule comprising a breakpoint.

Certain aspects of the present disclosure relate to methods of treating or delaying progression of cancer, comprising: detecting in a sample from an individual having a cancer a fusion nucleic acid molecule of the present disclosure, or a fusion polypeptide encoded by the fusion nucleic acid molecule; and administering to the individual an effective amount of a treatment that comprises an anti-cancer therapy.

In some embodiments according to any of the embodiments described herein, the fusion nucleic acid molecule is an ALK fusion nucleic acid molecule of the present disclosure, e.g., (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule. Exemplary and non-limiting ALK fusion nucleic acid molecules are described in Table 1.

In some embodiments according to any of the embodiments described herein, the fusion nucleic acid molecule is an NTRK1 or NTRK3 fusion nucleic acid molecule of the present disclosure, e.g., (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule. Exemplary and non-limiting NTRK1 and NTRK3 fusion nucleic acid molecules are described in Table 1.

### Fusion Nucleic Acid Molecules and Polypeptides

Certain aspects of the present disclosure relate to fusion nucleic acid molecules, or fusion polypeptides encoded by the fusion nucleic acid molecules. In some embodiments, the fusion nucleic acid molecule is an ALK, NTRK1, or NTRK3 fusion nucleic acid molecule. In some embodiments, the fusion polypeptide is an ALK, NTRK1, or NTRK3 fusion polypeptide, e.g., encoded by an ALK, NTRK1, or NTRK3 fusion nucleic acid molecule disclosed herein. Exemplary and non-limiting ALK, NTRK1, and NTRK3 fusion nucleic acid molecules are described in Table 1. Exemplary and non-limiting ALK, NTRK1, and NTRK3 fusion polypeptides are those encoded by a fusion nucleic acid molecule described in Table 1.

TABLE 1
ALK and NTRK1/3 fusion nucleic acids

| | Detected | | 5′ breakpoint/ | | 3′ breakpoint/ |
|---|---|---|---|---|---|
| Cancer | from | 5′ gene | Exons included | 3′ gene | Exons included |
| uterus | DNA/RNA | NRP2 | ex1-8 | ALK | ex19-29 |
| leiomyosarcoma | | | | | |
| soft tissue | DNA/RNA | NRP2 | ex1-8 | ALK | ex19-29 |
| inflammatory | | | | | |
| myofibroblastic | | | | | |
| tumor | | | | | |
| bone osteosarcoma | DNA | PDE3A | ex1-10 | ALK | ex8*-29 |
| bone osteosarcoma | RNA | PSMD14 | ex1-1 | ALK | ex4-29 |
| uterus | RNA | SFT2D1 | ex1-1 | ALK | ex6-29 |
| leiomyosarcoma | | | | | |
| soft tissue | RNA | SLC37A3 | ex1-3 | ALK | ex4-29 |
| leiomyosarcoma | | | | | |
| soft tissue sarcoma | RNA | TANGO6 | ex1-1 | ALK | ex2-29 |
| undifferentiated | | | | | |
| soft tissue | RNA | WDR92 | ex1-7 | ALK | ex2-29 |
| leiomyosarcoma | | | | | |
| soft tissue malignant | DNA | GPA33 | ex1-4 | NTRK1 | ex5-17 |
| peripheral nerve | | | | | |
| sheath tumor | | | | | |
| (mpnst) | | | | | |
| soft tissue sarcoma | DNA | FAM19A2 | ex1-1 | NTRK1 | ex4-17 |
| (nos) | | | | | |
| soft tissue | DNA/RNA | CPSF6 | ex1-7 | NTRK1 | ex12-17 |
| liposarcoma | | | | | |
| soft tissue | DNA/RNA | SUCO | ex1-10 | NTRK1 | ex3-17 |
| liposarcoma | | | | | |
| uterus sarcoma (nos) | DNA/RNA | CACYBP | ex1-2 | NTRK1 | ex9-17 |
| soft tissue | RNA | ZNF382 | ex1-4 | NTRK1 | ex2-17 |
| leiomyosarcoma | | | | | |
| soft tissue | RNA | NDE1 | ex1-6 | NTRK3 | ex14-19 |
| myxofibrosarcoma | | | | | |
| soft tissue | DNA | DGCR5 | ex1-1 | NTRK3 | ex3-19 |
| leiomyosarcoma | | | | | |
| soft tissue sarcoma | RNA | UBE2Q2P1 | ex1-5 | NTRK3 | ex6-19 |
| (nos) | | | | | |

### ALK Fusion Nucleic Acid Molecules/Polypeptides

In some embodiments, the fusion nucleic acid molecule is an ALK fusion nucleic acid molecule of the present disclosure, e.g., (a) a neuropilin 2 (NRP2)-anaplastic lymphoma kinase (ALK) fusion nucleic acid molecule; (b) a phosphodiesterase 3A (PDE3A)-anaplastic lymphoma kinase (ALK) fusion nucleic acid molecule; (c) a proteasome 26S subunit, non-ATPase 14 (PSMD14)-anaplastic lymphoma kinase (ALK) fusion nucleic acid molecule; (d) an SFT2 domain containing 1 (SFT2D1)-anaplastic lymphoma kinase (ALK) fusion nucleic acid molecule; (e) a solute carrier family 37 member 3 (SLC37A3)-anaplastic lymphoma kinase (ALK) fusion nucleic acid molecule; (f) a transport and golgi organization 6 homolog (TANGO6)-anaplastic lymphoma kinase (ALK) fusion nucleic acid molecule; or (g) a WD repeat-containing protein 92 (WDR92)-anaplastic lymphoma kinase (ALK) fusion nucleic acid molecule. Exemplary and non-limiting ALK fusion nucleic acid molecules are described in Table 1.

As used herein “anaplastic lymphoma kinase” or “ALK” refer to a gene encoding an ALK mRNA or polypeptide. The ALK gene encodes the ALK receptor tyrosine kinase protein. ALK is also known as CD246, NBLST3, anaplastic lymphoma receptor tyrosine kinase, and ALK receptor tyrosine kinase. In some embodiments, an ALK gene is a human ALK gene. An exemplary ALK gene is represented by NCBI Gene ID No. 238. An exemplary ALK mRNA sequence is represented by NCBI Ref. Seq. NM_004304, provided below as SEQ ID NO: 1. An exemplary amino acid sequence of an ALK polypeptide is represented by NCBI Ref. Seq. NP_004295.

(SEQ ID NO: 1)
AGATGCGATCCAGCGGCTCTGGGGGCGGCAGCGGTGGTAGCAGCTGGTACCTCCCGCCGCCTCTGTTC
GG
AGGGTCGCGGGGCACCGAGGTGCTTTCCGGCCGCCCTCTGGTCGGCCACCCAAAGCCGCGGGCGCTGA
TG
ATGGGTGAGGAGGGGGCGGCAAGATTTCGGGCGCCCCTGCCCTGAACGCCCTCAGCTGCTGCCGCCGG
GG
CCGCTCCAGTGCCTGCGAACTCTGAGGAGCCGAGGCGCCGGTGAGAGCAAGGACGCTGCAAACTTGCG
CA
GCGCGGGGGCTGGGATTCACGCCCAGAAGTTCAGCAGGCAGACAGTCCGAAGCCTTCCCGCAGCGGAG
AG
ATAGCTTGAGGGTGCGCAAGACGGCAGCCTCCGCCCTCGGTTCCCGCCCAGACCGGGCAGAAGAGCTT
GG
AGGAGCCAAAAGGAACGCAAAAGGCGGCCAGGACAGCGTGCAGCAGCTGGGAGCCGCCGTTCTCAGCC
TT
AAAAGTTGCAGAGATTGGAGGCTGCCCCGAGAGGGGACAGACCCCAGCTCCGACTGCGGGGGGCAGGA
GA
GGACGGTACCCAACTGCCACCTCCCTTCAACCATAGTAGTTCCTCTGTACCGAGCGCAGCGAGCTACA
GA
CGGGGGCGCGGCACTCGGCGCGGAGAGCGGGAGGCTCAAGGTCCCAGCCAGTGAGCCCAGTGTGCTTG
AG
TGTCTCTGGACTCGCCCCTGAGCTTCCAGGTCTGTTTCATTTAGACTCCTGCTCGCCTCCGTGCAGTT
GG
GGGAAAGCAAGAGACTTGCGCGCACGCACAGTCCTCTGGAGATCAGGTGGAAGGAGCCGCTGGGTACC
AA
GGACTGTTCAGAGCCTCTTCCCATCTCGGGGAGAGCGAAGGGTGAGGCTGGGCCCGGAGAGCAGTGTA
AA
CGGCCTCCTCCGGCGGGATGGGAGCCATCGGGCTCCTGTGGCTCCTGCCGCTGCTGCTTTCCACGGCA
GC
TGTGGGCTCCGGGATGGGGACCGGCCAGCGCGCGGGCTCCCCAGCTGCGGGGCCGCCGCTGCAGCCCC
GG
GAGCCACTCAGCTACTCGCGCCTGCAGAGGAAGAGTCTGGCAGTTGACTTCGTGGTGCCCTCGCTCTT
CC
GTGTCTACGCCCGGGACCTACTGCTGCCACCATCCTCCTCGGAGCTGAAGGCTGGCAGGCCCGAGGCC
CG
CGGCTCGCTAGCTCTGGACTGCGCCCCGCTGCTCAGGTTGCTGGGGCCGGCGCCGGGGGTCTCCTGGA
CC
GCCGGTTCACCAGCCCCGGCAGAGGCCCGGACGCTGTCCAGGGTGCTGAAGGGCGGCTCCGTGCGCAA
GC
TCCGGCGTGCCAAGCAGTTGGTGCTGGAGCTGGGCGAGGAGGCGATCTTGGAGGGTTGCGTCGGGCCC
CC
CGGGGAGGCGGCTGTGGGGCTGCTCCAGTTCAATCTCAGCGAGCTGTTCAGTTGGTGGATTCGCCAAG
GC
GAAGGGCGACTGAGGATCCGCCTGATGCCCGAGAAGAAGGCGTCGGAAGTGGGCAGAGAGGGAAGGCT
GT
CCGCGGCAATTCGCGCCTCCCAGCCCCGCCTTCTCTTCCAGATCTTCGGGACTGGTCATAGCTCCTTG
GA
ATCACCAACAAACATGCCTTCTCCTTCTCCTGATTATTTTACATGGAATCTCACCTGGATAATGAAAG
AC
TCCTTCCCTTTCCTGTCTCATCGCAGCCGATATGGTCTGGAGTGCAGCTTTGACTTCCCCTGTGAGCT
GG
AGTATTCCCCTCCACTGCATGACCTCAGGAACCAGAGCTGGTCCTGGCGCCGCATCCCCTCCGAGGAG
GC
CTCCCAGATGGACTTGCTGGATGGGCCTGGGGCAGAGCGTTCTAAGGAGATGCCCAGAGGCTCCTTTC
TC
CTTCTCAACACCTCAGCTGACTCCAAGCACACCATCCTGAGTCCGTGGATGAGGAGCAGCAGTGAGCA
CT
GCACACTGGCCGTCTCGGTGCACAGGCACCTGCAGCCCTCTGGAAGGTACATTGCCCAGCTGCTGCCC
CA
CAACGAGGCTGCAAGAGAGATCCTCCTGATGCCCACTCCAGGGAAGCATGGTTGGACAGTGCTCCAGG
GA
AGAATCGGGCGTCCAGACAACCCATTTCGAGTGGCCCTGGAATACATCTCCAGTGGAAACCGCAGCTT
GT
CTGCAGTGGACTTCTTTGCCCTGAAGAACTGCAGTGAAGGAACATCCCCAGGCTCCAAGATGGCCCTG
CA
GAGCTCCTTCACTTGTTGGAATGGGACAGTCCTCCAGCTTGGGCAGGCCTGTGACTTCCACCAGGACT
GT
GCCCAGGGAGAAGATGAGAGCCAGATGTGCCGGAAACTGCCTGTGGGTTTTTACTGCAACTTTGAAGA
TG
GCTTCTGTGGCTGGACCCAAGGCACACTGTCACCCCACACTCCTCAATGGCAGGTCAGGACCCTAAAG
GA
TGCCCGGTTCCAGGACCACCAAGACCATGCTCTATTGCTCAGTACCACTGATGTCCCCGCTTCTGAAA
GT
GCTACAGTGACCAGTGCTACGTTTCCTGCACCGATCAAGAGCTCTCCATGTGAGCTCCGAATGTCCTG
GC
TCATTCGTGGAGTCTTGAGGGGAAACGTGTCCTTGGTGCTAGTGGAGAACAAAACCGGGAAGGAGCAA
GG
CAGGATGGTCTGGCATGTCGCCGCCTATGAAGGCTTGAGCCTGTGGCAGTGGATGGTGTTGCCTCTCC
TC
GATGTGTCTGACAGGTTCTGGCTGCAGATGGTCGCATGGTGGGGACAAGGATCCAGAGCCATCGTGGC
TT
TTGACAATATCTCCATCAGCCTGGACTGCTACCTCACCATTAGCGGAGAGGACAAGATCCTGCAGAAT
AC
AGCACCCAAATCAAGAAACCTGTTTGAGAGAAACCCAAACAAGGAGCTGAAACCCGGGGAAAATTCAC
CA
AGACAGACCCCCATCTTTGACCCTACAGTTCATTGGCTGTTCACCACATGTGGGGCCAGCGGGCCCCA
TG
GCCCCACCCAGGCACAGTGCAACAACGCCTACCAGAACTCCAACCTGAGCGTGGAGGTGGGGAGCGAG
GG
CCCCCTGAAAGGCATCCAGATCTGGAAGGTGCCAGCCACCGACACCTACAGCATCTCGGGCTACGGAG
CT
GCTGGCGGGAAAGGCGGGAAGAACACCATGATGCGGTCCCACGGCGTGTCTGTGCTGGGCATCTTCAA
CC
TGGAGAAGGATGACATGCTGTACATCCTGGTTGGGCAGCAGGGAGAGGACGCCTGCCCCAGTACAAAC
CA
GTTAATCCAGAAAGTCTGCATTGGAGAGAACAATGTGATAGAAGAAGAAATCCGTGTGAACAGAAGCG
TG
CATGAGTGGGCAGGAGGCGGAGGAGGAGGGGGTGGAGCCACCTACGTATTTAAGATGAAGGATGGAGT
GC
CGGTGCCCCTGATCATTGCAGCCGGAGGTGGTGGCAGGGCCTACGGGGCCAAGACAGACACGTTCCAC
CC
AGAGAGACTGGAGAATAACTCCTCGGTTCTAGGGCTAAACGGCAATTCCGGAGCCGCAGGTGGTGGAG
GT
GGCTGGAATGATAACACTTCCTTGCTCTGGGCCGGAAAATCTTTGCAGGAGGGTGCCACCGGAGGACA
TT
CCTGCCCCCAGGCCATGAAGAAGTGGGGGTGGGAGACAAGAGGGGGTTTCGGAGGGGGTGGAGGGGGG
TG
CTCCTCAGGTGGAGGAGGCGGAGGATATATAGGCGGCAATGCAGCCTCAAACAATGACCCCGAAATGG
AT
GGGGAAGATGGGGTTTCCTTCATCAGTCCACTGGGCATCCTGTACACCCCAGCTTTAAAAGTGATGGA
AG
GCCACGGGGAAGTGAATATTAAGCATTATCTAAACTGCAGTCACTGTGAGGTAGACGAATGTCACATG
GA
CCCTGAAAGCCACAAGGTCATCTGCTTCTGTGACCACGGGACGGTGCTGGCTGAGGATGGCGTCTCCT
GC
ATTGTGTCACCCACCCCGGAGCCACACCTGCCACTCTCGCTGATCCTCTCTGTGGTGACCTCTGCCCT
CG
TGGCCGCCCTGGTCCTGGCTTTCTCCGGCATCATGATTGTGTACCGCCGGAAGCACCAGGAGCTGCAA
GC
CATGCAGATGGAGCTGCAGAGCCCTGAGTACAAGCTGAGCAAGCTCCGCACCTCGACCATCATGACCG
AC
TACAACCCCAACTACTGCTTTGCTGGCAAGACCTCCTCCATCAGTGACCTGAAGGAGGTGCCGCGGAA
AA
ACATCACCCTCATTCGGGGTCTGGGCCATGGCGCCTTTGGGGAGGTGTATGAAGGCCAGGTGTCCGGA
AT
GCCCAACGACCCAAGCCCCCTGCAAGTGGCTGTGAAGACGCTGCCTGAAGTGTGCTCTGAACAGGACG
AA
CTGGATTTCCTCATGGAAGCCCTGATCATCAGCAAATTCAACCACCAGAACATTGTTCGCTGCATTGG
GG
TGAGCCTGCAATCCCTGCCCCGGTTCATCCTGCTGGAGCTCATGGCGGGGGGAGACCTCAAGTCCTTC
CT
CCGAGAGACCCGCCCTCGCCCGAGCCAGCCCTCCTCCCTGGCCATGCTGGACCTTCTGCACGTGGCTC
GG
GACATTGCCTGTGGCTGTCAGTATTTGGAGGAAAACCACTTCATCCACCGAGACATTGCTGCCAGAAA
CT
GCCTCTTGACCTGTCCAGGCCCTGGAAGAGTGGCCAAGATTGGAGACTTCGGGATGGCCCGAGACATC
TA
CAGGGCGAGCTACTATAGAAAGGGAGGCTGTGCCATGCTGCCAGTTAAGTGGATGCCCCCAGAGGCCT
TC
ATGGAAGGAATATTCACTTCTAAAACAGACACATGGTCCTTTGGAGTGCTGCTATGGGAAATCTTTTC
TC
TTGGATATATGCCATACCCCAGCAAAAGCAACCAGGAAGTTCTGGAGTTTGTCACCAGTGGAGGCCGG
AT
GGACCCACCCAAGAACTGCCCTGGGCCTGTATACCGGATAATGACTCAGTGCTGGCAACATCAGCCTG
AA
GACAGGCCCAACTTTGCCATCATTTTGGAGAGGATTGAATACTGCACCCAGGACCCGGATGTAATCAA
CA
CCGCTTTGCCGATAGAATATGGTCCACTTGTGGAAGAGGAAGAGAAAGTGCCTGTGAGGCCCAAGGAC
CC
TGAGGGGGTTCCTCCTCTCCTGGTCTCTCAACAGGCAAAACGGGAGGAGGAGCGCAGCCCAGCTGCCC
CA
CCACCTCTGCCTACCACCTCCTCTGGCAAGGCTGCAAAGAAACCCACAGCTGCAGAGATCTCTGTTCG
AG
TCCCTAGAGGGCCGGCCGTGGAAGGGGGACACGTGAATATGGCATTCTCTCAGTCCAACCCTCCTTCG
GA
GTTGCACAAGGTCCACGGATCCAGAAACAAGCCCACCAGCTTGTGGAACCCAACGTACGGCTCCTGGT
TT
ACAGAGAAACCCACCAAAAAGAATAATCCTATAGCAAAGAAGGAGCCACACGACAGGGGTAACCTGGG
GC
TGGAGGGAAGCTGTACTGTCCCACCTAACGTTGCAACTGGGAGACTTCCGGGGGCCTCACTGCTCCTA
GA
GCCCTCTTCGCTGACTGCCAATATGAAGGAGGTACCTCTGTTCAGGCTACGTCACTTCCCTTGTGGGA
AT
GTCAATTACGGCTACCAGCAACAGGGCTTGCCCTTAGAAGCCGCTACTGCCCCTGGAGCTGGTCATTA
CG
AGGATACCATTCTGAAAAGCAAGAATAGCATGAACCAGCCTGGGCCCTGAGCTCGGTCGCACACTCAC
TT
CTCTTCCTTGGGATCCCTAAGACCGTGGAGGAGAGAGAGGCAATGGCTCCTTCACAAACCAGAGACCA
AA
TGTCACGTTTTGTTTTGTGCCAACCTATTTTGAAGTACCACCAAAAAAGCTGTATTTTGAAAATGCTT
TA
GAAAGGTTTTGAGCATGGGTTCATCCTATTCTTTCGAAAGAAGAAAATATCATAAAAATGAGTGATAA
AT
ACAAGGCCCAGATGTGGTTGCATAAGGTTTTTATGCATGTTTGTTGTATACTTCCTTATGCTTCTTTC
AA
ATTGTGTGTGCTCTGCTTCAATGTAGTCAGAATTAGCTGCTTCTATGTTTCATAGTTGGGGTCATAGA
TG
TTTCCTTGCCTTGTTGATGTGGACATGAGCCATTTGAGGGGAGAGGGAACGGAAATAAAGGAGTTATT
TG
TAATGACTAA

In some embodiments, the fusion nucleic acid molecule is a fusion nucleic acid molecule listed in Table 1, e.g., an ALK fusion molecule listed in Table 1. In some embodiments, the fusion nucleic acid molecule comprises a 5′ and/or 3′ breakpoint listed in Table 1. In some embodiments, the fusion nucleic acid molecule comprises a fusion junction listed in Table 1. In some embodiments, the fusion nucleic acid molecule encodes a fusion polypeptide having ALK kinase activity. In some embodiments, the kinase activity is constitutive. In some embodiments, the ALK fusion polypeptide is oncogenic. In some embodiments, the ALK fusion polypeptide promotes cancer cell survival, angiogenesis, cancer cell proliferation, and any combination thereof.

In some embodiments, a fusion nucleic acid molecule, or a fusion polypeptide encoded by the fusion nucleic acid molecule, comprises at least a portion of an ALK gene, and at least a portion of another gene, e.g., a neuropilin 2 (NRP2) gene, a phosphodiesterase 3A (PDE3A) gene, a proteasome 26S subunit, non-ATPase 14 (PSMD14) gene, an SFT2 domain containing 1 (SFT2D1) gene, a solute carrier family 37 member 3 (SLC37A3) gene, a transport and golgi organization 6 homolog (TANGO6) gene, or a WD repeat-containing protein 92 (WDR92) gene. For example, in some embodiments, the ALK fusion nucleic acid molecule is selected from NRP2-ALK, PDE3A-ALK, PSMD14-ALK, SFT2D1-ALK, SLC37A3-ALK, TANGO6-ALK, or WDR92-ALK. In some embodiments, the order of the genes is in the 5′ to 3′ direction. Exemplary and non-limiting ALK fusion nucleic acid molecules are described in Table 1 and/or in the Examples herein.

In some embodiments, the fusion nucleic acid molecule is an NRP2-ALK fusion nucleic acid molecule. As used herein “NRP2” refers to a gene encoding an NRP2 mRNA or polypeptide. The NRP2 gene encodes the neuropilin 2 receptor protein. NRP2 is also known as NP2, NPN2, PR02714, and VEGF165R2. In some embodiments, an NRP2 gene is a human NRP2 gene. An exemplary NRP2 gene is represented by NCBI Gene ID No. 8828. An exemplary NRP2 mRNA sequence is represented by NCBI Ref. Seq. NM_003872. An exemplary amino acid sequence of an NRP2 polypeptide is represented by NCBI Ref. Seq. NP_003863. In some embodiments, the fusion nucleic acid molecule comprises a 5′ breakpoint between exons 8 and 9 of NRP2. In some embodiments, the fusion nucleic acid molecule comprises exons 1-8 of NRP2. In some embodiments, the fusion nucleic acid molecule comprises a 3′ breakpoint between exons 18 and 19 of ALK. In some embodiments, the fusion nucleic acid molecule comprises exons 19-29 of ALK. In some embodiments, the cancer is a uterus leiomyosarcoma or soft tissue inflammatory myofibroblastic tumor.

In some embodiments, the fusion nucleic acid molecule is a PDE3A-ALK fusion nucleic acid molecule. As used herein “PDE3A” refers to a gene encoding a PDE3A mRNA or polypeptide. The PDE3A gene encodes the phosphodiesterase 3A. PDE3A is also known as HTNB, CGI-PDE, CGI-PDE a, and CGI-PDE-A. In some embodiments, a PDE3A gene is a human PDE3A gene. An exemplary PDE3A gene is represented by NCBI Gene ID No. 5139. An exemplary PDE3A mRNA sequence is represented by NCBI Ref. Seq. NM_000921. An exemplary amino acid sequence of a PDE3A polypeptide is represented by NCBI Ref. Seq. NP_000912. In some embodiments, the fusion nucleic acid molecule comprises a 5′ breakpoint between exons 10 and 11 of PDE3A. In some embodiments, the fusion nucleic acid molecule comprises exons 1-10 of PDE3A. In some embodiments, the fusion nucleic acid molecule comprises a 3′ breakpoint between exons 7 and 8 of ALK. In some embodiments, the fusion nucleic acid molecule comprises exons 8-29 of ALK. In some embodiments, the cancer is a bone osteosarcoma.

In some embodiments, the fusion nucleic acid molecule is a PSMD14-ALK fusion nucleic acid molecule. As used herein “PSMD14” refers to a gene encoding a PSMD14 mRNA or polypeptide. The PSMD14 gene encodes the proteasome 26S subunit, non-ATPase 14. PSMD14 is also known as PAD1, POH1, and RPN11. In some embodiments, a PSMD14 gene is a human PSMD14 gene. An exemplary PSMD14 gene is represented by NCBI Gene ID No. 10213. An exemplary PSMD14 mRNA sequence is represented by NCBI Ref. Seq. NM_005805. An exemplary amino acid sequence of a PSMD14 polypeptide is represented by NCBI Ref. Seq. NP_005796. In some embodiments, the fusion nucleic acid molecule comprises a 5′ breakpoint between exons 1 and 2 of PSMD14. In some embodiments, the fusion nucleic acid molecule comprises exon 1 of PSMD14. In some embodiments, the fusion nucleic acid molecule comprises a 3′ breakpoint between exons 3 and 4 of ALK. In some embodiments, the fusion nucleic acid molecule comprises exons 4-29 of ALK. In some embodiments, the cancer is a bone osteosarcoma.

In some embodiments, the fusion nucleic acid molecule is an SFT2D1-ALK fusion nucleic acid molecule. As used herein “SFT2D1” refers to a gene encoding a SFT2D1 mRNA or polypeptide. The SFT2D1 gene encodes the SFT2 domain containing 1 protein. SFT2D1 is also known as pRGR1 and C6orf83. In some embodiments, a SFT2D1 gene is a human SFT2D1 gene. An exemplary SFT2D1 gene is represented by NCBI Gene ID No. 113402. An exemplary SFT2D1 mRNA sequence is represented by NCBI Ref. Seq. NM_145169. An exemplary amino acid sequence of an SFT2D1 polypeptide is represented by NCBI Ref. Seq. NP_660152. In some embodiments, the fusion nucleic acid molecule comprises a 5′ breakpoint between exons 1 and 2 of SFT2D1. In some embodiments, the fusion nucleic acid molecule comprises exon 1 of SFT2D1. In some embodiments, the fusion nucleic acid molecule comprises a 3′ breakpoint between exons 5 and 6 of ALK. In some embodiments, the fusion nucleic acid molecule comprises exons 6-29 of ALK. In some embodiments, the cancer is a uterus leiomyosarcoma.

In some embodiments, the fusion nucleic acid molecule is an SLC37A3-ALK fusion nucleic acid molecule. As used herein “SLC37A3” refers to a gene encoding a SLC37A3 mRNA or polypeptide. The SLC37A3 gene encodes the solute carrier family 37 member 3 protein. SFT2D1 is also known as SPX3. In some embodiments, a SLC37A3 gene is a human SLC37A3 gene. An exemplary SLC37A3 gene is represented by NCBI Gene ID No. 84255. An exemplary SLC37A3 mRNA sequence is represented by NCBI Ref. Seq. NM_001287498. An exemplary amino acid sequence of an SLC37A3 polypeptide is represented by NCBI Ref. Seq. NP_001274427. In some embodiments, the fusion nucleic acid molecule comprises a 5′ breakpoint between exons 3 and 4 of SLC37A3. In some embodiments, the fusion nucleic acid molecule comprises exons 1-3 of SLC37A3. In some embodiments, the fusion nucleic acid molecule comprises a 3′ breakpoint between exons 3 and 4 of ALK. In some embodiments, the fusion nucleic acid molecule comprises exons 4-29 of ALK. In some embodiments, the cancer is a soft tissue leiomyosarcoma.

In some embodiments, the fusion nucleic acid molecule is a TANGO6-ALK fusion nucleic acid molecule. As used herein “TANGO6” refers to a gene encoding a TANGO6 mRNA or polypeptide. The TANGO6 gene encodes the transport and golgi organization 6 homolog. TANGO6 is also known as TMCO7. In some embodiments, a TANGO6 gene is a human TANGO6 gene. An exemplary TANGO6 gene is represented by NCBI Gene ID No. 79613. An exemplary TANGO6 mRNA sequence is represented by NCBI Ref. Seq. NM_024562. An exemplary amino acid sequence of a TANGO6 polypeptide is represented by NCBI Ref. Seq. NP_078838. In some embodiments, the fusion nucleic acid molecule comprises a 5′ breakpoint between exons 1 and 2 of TANGO6. In some embodiments, the fusion nucleic acid molecule comprises exon 1 of TANGO6. In some embodiments, the fusion nucleic acid molecule comprises a 3′ breakpoint between exons 1 and 2 of ALK. In some embodiments, the fusion nucleic acid molecule comprises exons 2-29 of ALK. In some embodiments, the cancer is a soft tissue undifferentiated cancer/tumor.

In some embodiments, the fusion nucleic acid molecule is a WDR92-ALK fusion nucleic acid molecule. As used herein “WDR92” refers to a gene encoding a WDR92 mRNA or polypeptide. The WDR92 gene encodes the dynein axonemal assembly factor 10 protein. WDR92 is also known as DNAAF10. In some embodiments, a WDR92 gene is a human WDR92 gene. An exemplary WDR92 gene is represented by NCBI Gene ID No. 116143. An exemplary WDR92 mRNA sequence is represented by NCBI Ref. Seq. NM_001256476. An exemplary amino acid sequence of a WDR92 polypeptide is represented by NCBI Ref. Seq. NP_001243405. In some embodiments, the fusion nucleic acid molecule comprises a 5′ breakpoint between exons 7 and 8 of WDR92. In some embodiments, the fusion nucleic acid molecule comprises exons 1-7 of WDR92. In some embodiments, the fusion nucleic acid molecule comprises a 3′ breakpoint between exons 1 and 2 of ALK. In some embodiments, the fusion nucleic acid molecule comprises exons 2-29 of ALK. In some embodiments, the cancer is a soft tissue leiomyosarcoma.

### NTRK1/3 Fusion Nucleic Acid Molecules/Polypeptides

In some embodiments, the fusion nucleic acid molecule is an NTRK1 or NTRK3 fusion nucleic acid molecule of the present disclosure, e.g., (a) a glycoprotein A33 (GPA33)-neurotrophic receptor tyrosine kinase 1 (NTRK1) fusion nucleic acid molecule; (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule; (d) a SUN domain containing ossification factor (SUCO)-neurotrophic receptor tyrosine kinase 1 (NTRK1) fusion nucleic acid molecule; (e) a calcyclin binding protein (CACYBP)-neurotrophic receptor tyrosine kinase 1 (NTRK1) fusion nucleic acid molecule; (f) a zinc finger protein 382 (ZNF382)-neurotrophic receptor tyrosine kinase 1 (NTRK1) fusion nucleic acid molecule; (g) a nudE neurodevelopment protein 1 (NDE1)-neurotrophic receptor tyrosine kinase 3 (NTRK3) fusion nucleic acid molecule; (h) a DiGeorge syndrome critical region gene 5 (DGCR5)-neurotrophic receptor tyrosine kinase 3 (NTRK3) fusion nucleic acid molecule; or (i) a ubiquitin conjugating enzyme E2 Q2 pseudogene 1 (UBE2Q2P1)-neurotrophic receptor tyrosine kinase 3 (NTRK3) fusion nucleic acid molecule. Exemplary and non-limiting NTRK1 and NTRK3 fusion nucleic acid molecules are described in Table 1.

As used herein “neurotrophic receptor tyrosine kinase 1” or “NTRK1” refer to a gene encoding an NTRK1 mRNA or polypeptide. The NTRK1 gene encodes the NTRK1 receptor tyrosine kinase protein. NTRK1 is also known as MTC, TRK, TRK1, TRKA, Trk-A, and p140-TrkA. In some embodiments, an NTRK1 gene is a human NTRK1 gene. An exemplary NTRK1 gene is represented by NCBI Gene ID No. 4914. An exemplary NTRK1 mRNA sequence is represented by NCBI Ref. Seq. NM_001007792, provided below as SEQ ID NO: 2. An exemplary amino acid sequence of an NTRK1 polypeptide is represented by NCBI Ref. Seq. NP_001007793.

(SEQ ID NO: 2)
GCACCCTGGTCATCTGCGGACTCAGCCTGAGCTTCCAGAGGGCCTAGGAGCAGTAAGGGAGTGAGTGGGC
AACTCGGCGCATGAAGGAGGCCGCCCTCATCTGCCTGGCACCCTCTGTACCCCCGATCTTGACGGTGAAG
TCCTGGGACACCATGCAGTTGCGGGCTGCTAGATCTCGGTGCACAAACTTGTTGGCAGCAAGCTACATCG
AGAACCAGCAGCATCTGCAGCATCTGGAGCTCCGTGATCTGAGGGGCCTGGGGGAGCTGAGAAACCTCAC
CATCGTGAAGAGTGGTCTCCGTTTCGTGGCGCCAGATGCCTTCCATTTCACTCCTCGGCTCAGTCGCCTG
AATCTCTCCTTCAACGCTCTGGAGTCTCTCTCCTGGAAAACTGTGCAGGGCCTCTCCTTACAGGAACTGG
TCCTGTCGGGGAACCCTCTGCACTGTTCTTGTGCCCTGCGCTGGCTACAGCGCTGGGAGGAGGAGGGACT
GGGCGGAGTGCCTGAACAGAAGCTGCAGTGTCATGGGCAAGGGCCCCTGGCCCACATGCCCAATGCCAGC
TGTGGTGTGCCCACGCTGAAGGTCCAGGTGCCCAATGCCTCGGTGGATGTGGGGGACGACGTGCTGCTGC
GGTGCCAGGTGGAGGGGCGGGGCCTGGAGCAGGCCGGCTGGATCCTCACAGAGCTGGAGCAGTCAGCCAC
GGTGATGAAATCTGGGGGTCTGCCATCCCTGGGGCTGACCCTGGCCAATGTCACCAGTGACCTCAACAGG
AAGAACGTGACGTGCTGGGCAGAGAACGATGTGGGCCGGGCAGAGGTCTCTGTTCAGGTCAACGTCTCCT
TCCCGGCCAGTGTGCAGCTGCACACGGCGGTGGAGATGCACCACTGGTGCATCCCCTTCTCTGTGGATGG
GCAGCCGGCACCGTCTCTGCGCTGGCTCTTCAATGGCTCCGTGCTCAATGAGACCAGCTTCATCTTCACT
GAGTTCCTGGAGCCGGCAGCCAATGAGACCGTGCGGCACGGGTGTCTGCGCCTCAACCAGCCCACCCACG
TCAACAACGGCAACTACACGCTGCTGGCTGCCAACCCCTTCGGCCAGGCCTCCGCCTCCATCATGGCTGC
CTTCATGGACAACCCTTTCGAGTTCAACCCCGAGGACCCCATCCCTGACACTAACAGCACATCTGGAGAC
CCGGTGGAGAAGAAGGACGAAACACCTTTTGGGGTCTCGGTGGCTGTGGGCCTGGCCGTCTTTGCCTGCC
TCTTCCTTTCTACGCTGCTCCTTGTGCTCAACAAATGTGGACGGAGAAACAAGTTTGGGATCAACCGCCC
GGCTGTGCTGGCTCCAGAGGATGGGCTGGCCATGTCCCTGCATTTCATGACATTGGGTGGCAGCTCCCTG
TCCCCCACCGAGGGCAAAGGCTCTGGGCTCCAAGGCCACATCATCGAGAACCCACAATACTTCAGTGATG
CCTGTGTTCACCACATCAAGCGCCGGGACATCGTGCTCAAGTGGGAGCTGGGGGAGGGCGCCTTTGGGAA
GGTCTTCCTTGCTGAGTGCCACAACCTCCTGCCTGAGCAGGACAAGATGCTGGTGGCTGTCAAGGCACTG
AAGGAGGCGTCCGAGAGTGCTCGGCAGGACTTCCAGCGTGAGGCTGAGCTGCTCACCATGCTGCAGCACC
AGCACATCGTGCGCTTCTTCGGCGTCTGCACCGAGGGCCGCCCCCTGCTCATGGTCTTTGAGTATATGCG
GCACGGGGACCTCAACCGCTTCCTCCGATCCCATGGACCTGATGCCAAGCTGCTGGCTGGTGGGGAGGAT
GTGGCTCCAGGCCCCCTGGGTCTGGGGCAGCTGCTGGCCGTGGCTAGCCAGGTCGCTGCGGGGATGGTGT
ACCTGGCGGGTCTGCATTTTGTGCACCGGGACCTGGCCACACGCAACTGTCTAGTGGGCCAGGGACTGGT
GGTCAAGATTGGTGATTTTGGCATGAGCAGGGATATCTACAGCACCGACTATTACCGTGTGGGAGGCCGC
ACCATGCTGCCCATTCGCTGGATGCCGCCCGAGAGCATCCTGTACCGTAAGTTCACCACCGAGAGCGACG
TGTGGAGCTTCGGCGTGGTGCTCTGGGAGATCTTCACCTACGGCAAGCAGCCCTGGTACCAGCTCTCCAA
CACGGAGGCAATCGACTGCATCACGCAGGGACGTGAGTTGGAGCGGCCACGTGCCTGCCCACCAGAGGTC
TACGCCATCATGCGGGGCTGCTGGCAGCGGGAGCCCCAGCAACGCCACAGCATCAAGGATGTGCACGCCC
GGCTGCAAGCCCTGGCCCAGGCACCTCCTGTCTACCTGGATGTCCTGGGCTAGGGGGCCGGCCCAGGGGC
TGGGAGTGGTTAGCCGGAATACTGGGGCCTGCCCTCAGCATCCCCCATAGCTCCCAGCAGCCCCAGGGTG
ATCTCAAAGTATCTAATTCACCCTCAGCATGTGGGAAGGGACAGGTGGGGGCTGGGAGTAGAGGATGTTC
CTGCTTCTCTAGGCAAGGTCCCGTCATAGCAATTATATTTATTATCCCTTGAAAAAAAAAA

In some embodiments, the fusion nucleic acid molecule is a fusion nucleic acid molecule listed in Table 1, e.g., an NTRK1 fusion molecule listed in Table 1. In some embodiments, the fusion nucleic acid molecule comprises a 5′ and/or 3′ breakpoint listed in Table 1. In some embodiments, the fusion nucleic acid molecule comprises a fusion junction listed in Table 1. In some embodiments, the fusion nucleic acid molecule encodes a fusion polypeptide having NTRK1 kinase activity. In some embodiments, the kinase activity is constitutive. In some embodiments, the NTRK1 fusion polypeptide is oncogenic. In some embodiments, the NTRK1 fusion polypeptide promotes cancer cell survival, angiogenesis, cancer cell proliferation, and any combination thereof.

In some embodiments, a fusion nucleic acid molecule, or a fusion polypeptide encoded by the fusion nucleic acid molecule, comprises at least a portion of an NTRK1 gene, and at least a portion of another gene, e.g., a glycoprotein A33 (GPA33) gene, a family with sequence similarity 19 (chemokine (C-C motif)-like, member A2) (FAM19A2) gene, a cleavage and polyadenylation specific factor 6 (CPSF6) gene, a SUN domain containing ossification factor (SUCO) gene, a calcyclin binding protein (CACYBP) gene, or a zinc finger protein 382 (ZNF382) gene. For example, in some embodiments, the NTRK1 fusion nucleic acid molecule is selected from GPA33-NTRK1, FAM19A2-NTRK1, CPSF6-NTRK1, SUCO-NTRK1, CACYBP-NTRK1, or ZNF382-NTRK1. In some embodiments, the order of the genes is in the 5′ to 3′ direction. Exemplary and non-limiting NTRK1 fusion nucleic acid molecules are described in Table 1 and/or in the Examples herein.

In some embodiments, the fusion nucleic acid molecule is a GPA33-NTRK1 fusion nucleic acid molecule. As used herein “GPA33” refers to a gene encoding a GPA33 mRNA or polypeptide. The GPA33 gene encodes the glycoprotein A33. GPA33 is also known as A33. In some embodiments, a GPA33 gene is a human GPA33 gene. An exemplary GPA33 gene is represented by NCBI Gene ID No. 10223. An exemplary GPA33 mRNA sequence is represented by NCBI Ref. Seq. NM_005814. An exemplary amino acid sequence of a GPA33 polypeptide is represented by NCBI Ref. Seq. NP_005805. In some embodiments, the fusion nucleic acid molecule comprises a 5′ breakpoint between exons 4 and 5 of GPA33. In some embodiments, the fusion nucleic acid molecule comprises exons 1-4 of GPA33. In some embodiments, the fusion nucleic acid molecule comprises a 3′ breakpoint between exons 4 and 5 of NTRK1. In some embodiments, the fusion nucleic acid molecule comprises exons 5-17 of NTRK1. In some embodiments, the cancer is a soft tissue malignant peripheral nerve sheath tumor (MPNST).

In some embodiments, the fusion nucleic acid molecule is a FAM19A2-NTRK1 fusion nucleic acid molecule. As used herein “FAM19A2” refers to a gene encoding a FAM19A2 mRNA or polypeptide. The FAM19A2 gene encodes the TAFA chemokine like family member 2 protein. FAM19A2 is also known as TAFA-2 and TAFA2. In some embodiments, a FAM19A2 gene is a human FAM19A2 gene. An exemplary FAM19A2 gene is represented by NCBI Gene ID No. 338811. An exemplary FAM19A2 mRNA sequence is represented by NCBI Ref. Seq. NM_178539. An exemplary amino acid sequence of a FAM19A2 polypeptide is represented by NCBI Ref. Seq. NP_848634. In some embodiments, the fusion nucleic acid molecule comprises a 5′ breakpoint between exons 1 and 2 of FAM19A2. In some embodiments, the fusion nucleic acid molecule comprises exon 1 of FAM19A2. In some embodiments, the fusion nucleic acid molecule comprises a 3′ breakpoint between exons 3 and 4 of NTRK1. In some embodiments, the fusion nucleic acid molecule comprises exons 4-17 of NTRK1. In some embodiments, the cancer is a soft tissue sarcoma (nos).

In some embodiments, the fusion nucleic acid molecule is a CPSF6-NTRK1 fusion nucleic acid molecule. As used herein “CPSF6” refers to a gene encoding a CPSF6 mRNA or polypeptide. The CPSF6 gene encodes the cleavage and polyadenylation specific factor 6. CPSF6 is also known as CFIM, CFIM68, CFIM72, HPBRII-4, and HPBRII-7. In some embodiments, a CPSF6 gene is a human CPSF6 gene. An exemplary CPSF6 gene is represented by NCBI Gene ID No. 11052. An exemplary CPSF6 mRNA sequence is represented by NCBI Ref. Seq. NM_001300947. An exemplary amino acid sequence of a CPSF6 polypeptide is represented by NCBI Ref. Seq. NP_001287876. In some embodiments, the fusion nucleic acid molecule comprises a 5′ breakpoint between exons 7 and 8 of CPSF6. In some embodiments, the fusion nucleic acid molecule comprises exons 1-7 of CPSF6. In some embodiments, the fusion nucleic acid molecule comprises a 3′ breakpoint between exons 11 and 12 of NTRK1. In some embodiments, the fusion nucleic acid molecule comprises exons 12-17 of NTRK1. In some embodiments, the cancer is a soft tissue liposarcoma.

In some embodiments, the fusion nucleic acid molecule is a SUCO-NTRK1 fusion nucleic acid molecule. As used herein “SUCO” refers to a gene encoding a SUCO mRNA or polypeptide. The SUCO gene encodes the SUN domain containing ossification factor. SUCO is also known as CH1, OPT, SLP1, and C1orf9. In some embodiments, a SUCO gene is a human SUCO gene. An exemplary SUCO gene is represented by NCBI Gene ID No. 51430. An exemplary SUCO mRNA sequence is represented by NCBI Ref. Seq. NM_001282750. An exemplary amino acid sequence of a SUCO polypeptide is represented by NCBI Ref. Seq. NP_001269679. In some embodiments, the fusion nucleic acid molecule comprises a 5′ breakpoint between exons 10 and 11 of SUCO. In some embodiments, the fusion nucleic acid molecule comprises exons 1-10 of SUCO. In some embodiments, the fusion nucleic acid molecule comprises a 3′ breakpoint between exons 2 and 3 of NTRK1. In some embodiments, the fusion nucleic acid molecule comprises exons 3-17 of NTRK1. In some embodiments, the cancer is a soft tissue liposarcoma.

In some embodiments, the fusion nucleic acid molecule is a CACYBP-NTRK1 fusion nucleic acid molecule. As used herein “CACYBP” refers to a gene encoding a CACYBP mRNA or polypeptide. The CACYBP gene encodes the calcyclin binding protein. CACYBP is also known as SIP, GIGS, PNAS-107, and S100A6BP. In some embodiments, a CACYBP gene is a human CACYBP gene. An exemplary CACYBP gene is represented by NCBI Gene ID No. 27101. An exemplary CACYBP mRNA sequence is represented by NCBI Ref. Seq. NM_001007214. An exemplary amino acid sequence of a CACYBP polypeptide is represented by NCBI Ref. Seq. NP_001007215. In some embodiments, the fusion nucleic acid molecule comprises a 5′ breakpoint between exons 2 and 3 of CACYBP. In some embodiments, the fusion nucleic acid molecule comprises exons 1 and 2 of CACYBP. In some embodiments, the fusion nucleic acid molecule comprises a 3′ breakpoint between exons 8 and 9 of NTRK1. In some embodiments, the fusion nucleic acid molecule comprises exons 9-17 of NTRK1. In some embodiments, the cancer is a uterus sarcoma.

In some embodiments, the fusion nucleic acid molecule is a ZNF382-NTRK1 fusion nucleic acid molecule. As used herein “ZNF382” refers to a gene encoding a ZNF382 mRNA or polypeptide. The ZNF382 gene encodes the zinc finger 382 protein. ZNF382 is also known as KS1. In some embodiments, a ZNF382 gene is a human ZNF382 gene. An exemplary ZNF382 gene is represented by NCBI Gene ID No. 84911. An exemplary ZNF382 mRNA sequence is represented by NCBI Ref. Seq. NM_001256838. An exemplary amino acid sequence of a ZNF382 polypeptide is represented by NCBI Ref. Seq. NP_001243767. In some embodiments, the fusion nucleic acid molecule comprises a 5′ breakpoint between exons 4 and 5 of ZNF382. In some embodiments, the fusion nucleic acid molecule comprises exons 1-4 of ZNF382. In some embodiments, the fusion nucleic acid molecule comprises a 3′ breakpoint between exons 8 and 9 of NTRK1. In some embodiments, the fusion nucleic acid molecule comprises exons 9-17 of NTRK1. In some embodiments, the cancer is a soft tissue leiomyosarcoma.

As used herein “neurotrophic receptor tyrosine kinase 3” or “NTRK3” refer to a gene encoding an NTRK3 mRNA or polypeptide. The NTRK3 gene encodes the NTRK3 receptor tyrosine kinase protein. NTRK3 is also known as TRKC, GP145-TrkC, and gp145(trkC). In some embodiments, an NTRK3 gene is a human NTRK3 gene. An exemplary NTRK3 gene is represented by NCBI Gene ID No. 4916. An exemplary NTRK3 mRNA sequence is represented by NCBI Ref. Seq. NM_001007156, provided below as SEQ ID NO: 3. An exemplary amino acid sequence of an NTRK3 polypeptide is represented by NCBI Ref. Seq. NP_001007157.

(SEQ ID NO: 3)
GCACTTGTACATTTCTGCAGCCGCGCGGCGAGCCATTCGCGGCGGCTGCTGCAGCTCCTACTGCATCTTC
CTTCTCTTCCTTTCCTCGGGCTCCGGTCTCGGAGTCGGAGAGCGCGCCTCGCTTCCAGAGCCCCCGGACC
CGGCGAGTCAGCGATCGCCGAGCCGGCCACCATGCCCGGCAGACCGCGCCACTAGGCGCTCCTCGCGGCT
CCCACCCGGCGGCGGCGGCGGCGGCGGCGGCGTCCGCGATGGTTTCAGACGCTGAAGGATTTTGCATCTG
ATCGCTCGGCGTTTCAAAGAAGCAGCGATCGGAGATGGATGTCTCTCTTTGCCCAGCCAAGTGTAGTTTC
TGGCGGATTTTCTTGCTGGGAAGCGTCTGGCTGGACTATGTGGGCTCCGTGCTGGCTTGCCCTGCAAATT
GTGTCTGCAGCAAGACTGAGATCAATTGCCGGCGGCCGGACGATGGGAACCTCTTCCCCCTCCTGGAAGG
GCAGGATTCAGGGAACAGCAATGGGAACGCCAGTATCAACATCACGGACATCTCAAGGAATATCACTTCC
ATACACATAGAGAACTGGCGCAGTCTTCACACGCTCAACGCCGTGGACATGGAGCTCTACACCGGACTTC
AAAAGCTGACCATCAAGAACTCAGGACTTCGGAGCATTCAGCCCAGAGCCTTTGCCAAGAACCCCCATTT
GCGTTATATAAACCTGTCAAGTAACCGGCTCACCACACTCTCGTGGCAGCTCTTCCAGACGCTGAGTCTT
CGGGAATTGCAGTTGGAGCAGAACTTTTTCAACTGCAGCTGTGACATCCGCTGGATGCAGCTCTGGCAGG
AGCAGGGGGAGGCCAAGCTCAACAGCCAGAACCTCTACTGCATCAACGCTGATGGCTCCCAGCTTCCTCT
CTTCCGCATGAACATCAGTCAGTGTGACCTTCCTGAGATCAGCGTGAGCCACGTCAACCTGACCGTACGA
GAGGGTGACAACGCTGTTATCACTTGCAATGGCTCTGGATCACCCCTTCCTGATGTGGACTGGATAGTCA
CTGGGCTGCAGTCCATCAACACTCACCAGACCAATCTGAACTGGACCAATGTTCATGCCATCAACTTGAC
GCTGGTGAATGTGACGAGTGAGGACAATGGCTTCACCCTGACGTGCATTGCAGAGAACGTGGTGGGCATG
AGCAATGCCAGTGTTGCCCTCACTGTCTACTATCCCCCACGTGTGGTGAGCCTGGAGGAGCCTGAGCTGC
GCCTGGAGCACTGCATCGAGTTTGTGGTGCGTGGCAACCCCCCACCAACGCTGCACTGGCTGCACAATGG
GCAGCCTCTGCGGGAGTCCAAGATCATCCATGTGGAATACTACCAAGAGGGAGAGATTTCCGAGGGCTGC
CTGCTCTTCAACAAGCCCACCCACTACAACAATGGCAACTATACCCTCATTGCCAAAAACCCACTGGGCA
CAGCCAACCAGACCATCAATGGCCACTTCCTCAAGGAGCCCTTTCCAGAGAGCACGGATAACTTTATCTT
GTTTGACGAAGTGAGTCCCACACCTCCTATCACTGTGACCCACAAACCAGAAGAAGACACTTTTGGGGTA
TCCATAGCAGTTGGACTTGCTGCTTTTGCCTGTGTCCTGTTGGTGGTTCTCTTCGTCATGATCAACAAAT
ATGGTCGACGGTCCAAATTTGGAATGAAGGGTCCCGTGGCTGTCATCAGTGGTGAGGAGGACTCAGCCAG
CCCACTGCACCACATCAACCACGGCATCACCACGCCCTCGTCACTGGATGCCGGGCCCGACACTGTGGTC
ATTGGCATGACTCGCATCCCTGTCATTGAGAACCCCCAGTACTTCCGTCAGGGACACAACTGCCACAAGC
CGGACACGTGGGTCTTTTCAAACATAGACAATCATGGGATATTAAACTTGAAGGACAATAGAGATCATCT
AGTCCCATCAACTCACTATATATATGAGGAACCTGAGGTCCAGAGTGGGGAAGTGTCTTACCCAAGGTCA
CATGGTTTCAGAGAAATTATGTTGAATCCAATAAGCCTTCCCGGACATTCCAAGCCTCTTAACCATGGCA
TCTATGTTGAGGATGTCAATGTTTATTTCAGCAAAGGACGTCATGGCTTTTAAAAACTCCTTTTAAGCCT
CCTTGTTTTGATGTCACCTTGGTAGGCTGGGCCCTCTGAGAGGTTGGAAGCTCTAGGCATTGTTCTCTTT
GGATCCAGGGATGCTAAGTAGAAACTGCATGAGCCACCAGTGCCCCGGCACCCTTTAACACCACCAGATG
GGTGTTTTCCCCCATCCACCACTGGCAGGGTTGCCCCTTCCCTCCAATCATCACTGTGCTCCTTTTTTCC
CGGCCTACGAGGCAGCTCCTGCCACTATCTTTAGAGCCAATAAAGAGAATTAAAAACCTGTGCACCAGGA
GCATCTTTTAAATACACTAGCCATTCTCTTGCTTTACAAAAACAACCTAACCATCACAAGAAAGCCTGAT
GAAGTCCAGCCGTGCTCCAGCCTCACTTTCCCTGCTTGGAAGCGTGGGGTCTCCCTGGCTCTCCCAGGAT
ACCATGCTGTCCTCTTAGTGACCTCGTCGCCCTGCAACCTCCAGTGGGGAAGAGTCACAGAGAGCACCTA
AGCAGAGGTGGAGACGGCGCGGTAAGAGGAGGGGGAGCCAGGCTCAAGTATTGGCACCAAGTTAGGTCTC
AGAGGAAAGAATGGAAACCAATCACTTTACATTTTTATTTTTATTTTCGGTGGAAAAATCATCCTTTTTT
GGGACATACTTGCCCCCTACTTCCTCTTCTCTCTGGAACGGCTCACAATGAGTGTGACATTAGAAAACTC
CTTGCAGAGGAGAGTTTCTCCAGGCTCTTCCTGGGCCCTTAGATCTGCAGTTCCGACAAGCTTTGGCTGC
AGGAGGTTTTACCCATGAACTGGCCATCCTACTAGGACCACAAGGGACCAAGGGAATCAGGGACAAAGGC
CCTTCCTGCCAGCCCATGATCCCGGGATTGGCTCTCTTCCCCTACTTCCACTTATTCTTGACTCTGAGAA
CTTTTGGAACCCAATGGAATCAGCATTTCAAGGTCAAGATGAACTGAAGGGGAAGAGAAGTAAAACTTGG
CCTCCTCCAGCCCCTCTCATGGCACCAATGGAAGTGTCCTCCTGTTCTCTGGTCAATATGTGTGTTTACT
TTGCTTGCTTTGACTCATGCCTTACTCCATGGCCACCCTCTCCCAAAGAGGGGGCTCGCTTCCCCCATTT
TCAACTTGATCCACTGAGGAGAGGGAAGGGGGTGACTTTCCCTTCTTCAGTAGGAAAGGCACATTTGTAG
GGCCTGAAACTCTCCCGTATTTGCTGACTCATTGGTGGAGTAGACTTCTGGCTCCCAGCTCCACTGGCCC
ATGGGGCCTCCATTGTATGAAGTCAGCATAGGCTGCCCACCTAATGGTGGAGAGCATGAAACTGGGAGCA
TCCTGTGGGGGGCTTGTGGGGGAAAAAGGTGGTTGTTTTAACCCACCGTTGTTTTGGGGTGGTGTTGCAC
ACTAGTAGAGAATAGAGTCTATGCCTTTGGCAAATTTAACTGGGAGTTTGGATTCCCACTTAAGGGTTTT
ACTTCTTGGGTCCTGTGGATGGTGGTTCTTCGTGTCAGGATCCCAGCCCGATTCTGCAAATGCCTCCATG
GGGTTTAAAAACATGAGGCTTTCCAAGTTCTTGCCCAGTATCTGGGGCAGCCTCCAGAGTATCACCTGGG
AGTTCAGGTTCTCTCCAGGGCTCCAGGTGTGTGTTTATCTCGCCCCCTCCAGCTCTCCTCATCCTGCTCC
CCATTGCTCCATGTCAGGCTGTTCCCCATTGTGCCCTGCTGATGCTTTGGGTCCAGGGCCTCCTCCCAAG
TGTGGCTTTAAGGAGTAAGCTTGAGGATGATGTTTTTTAATTATTGTAAATCATTACCTCATTTCCAGCC
TCCCAGGCTCCATCCATCCCAGCATCTTTTATTCTGCCATTTTCCTCACCTTGTGCTATGACAATGGGGC
GTTGTGTTTCCACAGAGACTTATAGGAGTGTTCAGTGTATAGTTTCTTAATAAACACTTTATTTTCTAAT
GAAA

In some embodiments, the fusion nucleic acid molecule is a fusion nucleic acid molecule listed in Table 1, e.g., an NTRK3 fusion molecule listed in Table 1. In some embodiments, the fusion nucleic acid molecule comprises a 5′ and/or 3′ breakpoint listed in Table 1. In some embodiments, the fusion nucleic acid molecule comprises a fusion junction listed in Table 1. In some embodiments, the fusion nucleic acid molecule encodes a fusion polypeptide having NTRK3 kinase activity. In some embodiments, the kinase activity is constitutive. In some embodiments, the NTRK3 fusion polypeptide is oncogenic. In some embodiments, the NTRK3 fusion polypeptide promotes cancer cell survival, angiogenesis, cancer cell proliferation, and any combination thereof.

In some embodiments, a fusion nucleic acid molecule, or a fusion polypeptide encoded by the fusion nucleic acid molecule, comprises at least a portion of an NTRK3 gene, and at least a portion of another gene, e.g., a nudE neurodevelopment protein 1 (NDE1) gene, a DiGeorge syndrome critical region gene 5 (DGCR5) gene, or a ubiquitin conjugating enzyme E2 Q2 pseudogene 1 (UBE2Q2P1) gene. For example, in some embodiments, the NTRK3 fusion nucleic acid molecule is selected from NDE1-NTRK3, DGCR5-NTRK3, or UBE2Q2P1-NTRK3. In some embodiments, the order of the genes is in the 5′ to 3′ direction. Exemplary and non-limiting NTRK3 fusion nucleic acid molecules are described in Table 1 and/or in the Examples herein.

In some embodiments, the fusion nucleic acid molecule is an NDE1-NTRK3 fusion nucleic acid molecule. As used herein “NDE1” refers to a gene encoding an NDE1 mRNA or polypeptide. The NDE1 gene encodes the nudE neurodevelopment protein 1. NDE1 is also known as NDE, LIS4, MHAC, NUDE, NUDE1, and HOM-TES-87. In some embodiments, an NDE1 gene is a human NDE1 gene. An exemplary NDE1 gene is represented by NCBI Gene ID No. 54820. An exemplary NDE1 mRNA sequence is represented by NCBI Ref. Seq. NM_001143979. An exemplary amino acid sequence of an NDE1 polypeptide is represented by NCBI Ref. Seq. NP_001137451. In some embodiments, the fusion nucleic acid molecule comprises a 5′ breakpoint between exons 6 and 7 of NDE1. In some embodiments, the fusion nucleic acid molecule comprises exons 1-6 of NDE1. In some embodiments, the fusion nucleic acid molecule comprises a 3′ breakpoint between exons 13 and 14 of NTRK3. In some embodiments, the fusion nucleic acid molecule comprises exons 14-19 of NTRK3. In some embodiments, the cancer is a soft tissue myxofibrosarcoma.

In some embodiments, the fusion nucleic acid molecule is a DGCR5-NTRK3 fusion nucleic acid molecule. As used herein “DGCR5” refers to a gene encoding a DGCR5mRNA or polypeptide. The DGCR5 gene encodes the DiGeorge syndrome critical region gene 5 ncRNA. DGCR5 is also known as DCR9, DGS-A, DGS-B, DGCR10, LINC00037, POM121L5P, and NCRNA0037. In some embodiments, a DGCR5 gene is a human DGCR5 gene. An exemplary DGCR5 gene is represented by NCBI Gene ID No. 26220. An exemplary DGCR5 RNA sequence is represented by NCBI Ref. Seq. NR_002733. In some embodiments, the fusion nucleic acid molecule comprises a 5′ breakpoint between exons 1 and 2 of DGCR5. In some embodiments, the fusion nucleic acid molecule comprises exon 1 of DGCR5. In some embodiments, the fusion nucleic acid molecule comprises a 3′ breakpoint between exons 2 and 3 of NTRK3. In some embodiments, the fusion nucleic acid molecule comprises exons 3-19 of NTRK3. In some embodiments, the cancer is a soft tissue leiomyosarcoma.

In some embodiments, the fusion nucleic acid molecule is a UBE2Q2P1-NTRK3 fusion nucleic acid molecule. As used herein “UBE2Q2P1” refers to a gene encoding a UBE2Q2P1 mRNA or polypeptide. The UBE2Q2P1 gene encodes the ubiquitin conjugating enzyme E2 Q2 pseudogene 1. UBE2Q2P1 is also known as UBE2QP1. In some embodiments, a UBE2Q2P1 gene is a human UBE2Q2P1 gene. An exemplary UBE2Q2P1 gene is represented by NCBI Gene ID No. 388165. An exemplary UBE2Q2P1 RNA sequence is represented by NCBI Ref. Seq. NR_003661. In some embodiments, the fusion nucleic acid molecule comprises a 5′ breakpoint between exons 5 and 6 of UBE2Q2P1. In some embodiments, the fusion nucleic acid molecule comprises exons 1-5 of UBE2Q2P1. In some embodiments, the fusion nucleic acid molecule comprises a 3′ breakpoint between exons 5 and 6 of NTRK3. In some embodiments, the fusion nucleic acid molecule comprises exons 6-19 of NTRK3. In some embodiments, the cancer is a soft tissue sarcoma (nos).

### Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; assessing a fusion nucleic acid molecule or polypeptide in a cancer in an individual; detecting the presence or absence of a cancer in an individual; monitoring progression or recurrence of a cancer in an individual; or identifying a candidate treatment for a cancer in an individual in need thereof.

Exemplary cancers to be treated by the methods of the present disclosure are those described in Table 1 and/or those harboring a fusion nucleic acid molecule described in Table 1, or harboring a fusion polypeptide encoding a fusion nucleic acid molecule described in Table 1.

In some embodiments, the cancer is a sarcoma. In some embodiments, the cancer is a uterus leiomyosarcoma, soft tissue inflammatory myofibroblastic tumor, soft tissue sarcoma (nos), bone osteosarcoma, soft tissue leiomyosarcoma, soft tissue sarcoma undifferentiated, soft tissue malignant peripheral nerve sheath tumor (mpnst), soft tissue liposarcoma, uterus sarcoma (nos), or soft tissue myxofibrosarcoma.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the cancer is appendix adenocarcinoma, bladder adenocarcinoma, bladder urothelial (transitional cell) carcinoma, breast cancer not otherwise specified (NOS), breast carcinoma NOS, breast invasive ductal carcinoma (IDC), breast invasive lobular carcinoma (ILC), cervix squamous cell carcinoma (SCC), colon adenocarcinoma (CRC), esophagus adenocarcinoma, esophagus carcinoma NOS, esophagus squamous cell carcinoma (SCC), eye intraocular melanoma, gallbladder adenocarcinoma, gastroesophageal junction adenocarcinoma, intra-hepatic cholangiocarcinoma, kidney cancer NOS, liver hepatocellular carcinoma (HCC), lung cancer NOS, lung adenocarcinoma, lung large cell carcinoma, lung non-small cell lung carcinoma (NSCLC) NOS, lung small cell undifferentiated carcinoma, lung squamous cell carcinoma (SCC), ovary cancer NOS, pancreas cancer NOS, pancreas ductal adenocarcinoma, pancreatobiliary carcinoma, prostate cancer NOS, prostate acinar adenocarcinoma, prostate ductal adenocarcinoma, rectum adenocarcinoma (CRC), skin melanoma, small intestine adenocarcinoma, soft tissue sarcoma NOS, stomach adenocarcinoma NOS, unknown primary cancer NOS, unknown primary adenocarcinoma, unknown primary carcinoma (CUP) NOS, unknown primary neuroendocrine tumor, unknown primary squamous cell carcinoma (SCC), or uterus endometrial adenocarcinoma NOS.

Certain aspects of the present disclosure relate to anti-cancer therapies, e.g., for treating or preventing progression of a cancer of the present disclosure.

In some embodiments, the anti-cancer therapy comprises an ALK-targeted therapy. For example, in some embodiments, the anti-cancer therapy comprises a kinase inhibitor. In some embodiments, the kinase inhibitor is a multi-kinase inhibitor or an ALK-specific inhibitor. In some embodiments, the kinase inhibitor is a tyrosine kinase inhibitor.

In some embodiments, the ALK-targeted therapy comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, the anti-cancer therapy comprises a kinase inhibitor. In some embodiments, the kinase inhibitor is a multi-kinase inhibitor or an ALK-specific inhibitor. In some embodiments, the kinase inhibitor is a tyrosine kinase inhibitor. In some embodiments, the kinase inhibitor inhibits kinase activity of an ALK polypeptide, e.g., an ALK fusion polypeptide described herein (including without limitation an ALK fusion polypeptide encoded by an ALK fusion nucleic acid listed in Table 1). In some embodiments, the kinase inhibitor is one or more of crizotinib, alectinib, ceritinib, lorlatinib, brigatinib, ensartinib (X-396), repotrectinib (TPX-0005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the kinase inhibitor is an ALK kinase inhibitor, e.g., as described in examples 3-39 of WO2005016894.

In some embodiments, the anti-cancer therapy comprises an NTRK1-targeted therapy and/or an NTRK3-targeted therapy. In some embodiments, the NTRK1 and/or NTRK3-targeted therapy comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for NTRK1/3-positive or NTRK1/3-rearranged cancer, an NTRK1/3-targeted therapy being tested in a clinical trial, a treatment for NTRK1/3-positive or NTRK1/3-rearranged cancer being tested in a clinical trial, or any combination thereof. In some embodiments, the anti-cancer therapy comprises a kinase inhibitor. In some embodiments, the anti-cancer therapy comprises a kinase inhibitor. In some embodiments, the kinase inhibitor is a multi-kinase inhibitor, an NTRK1-specific inhibitor, or an NTRK3-specific inhibitor. In some embodiments, the kinase inhibitor is a tyrosine kinase inhibitor. In some embodiments, the kinase inhibitor inhibits kinase activity of an NTRK1 or NTRK3 polypeptide, e.g., an NTRK1 or NTRK3 fusion polypeptide described herein (including without limitation an NTRK1 or NTRK3 fusion polypeptide encoded by an NTRK1 or NTRK3 fusion nucleic acid listed in Table 1). In some embodiments, the kinase inhibitor is one or more of AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), LOXO-195, a macrocyclic compound, ONO-5390556, oxindole 3, pegcantratinib (SNA-120), PHA-848125, PLX7486, a pyrazole derivative, a pyrazolo[1, 5a]pyrimidine, a pyridocarbazole, a pyridoquinazolinyl, a pyridotriazole, a pyrrolidinyl thiourea, a pyrrolidinyl urea, a pyrrolo[2, 3-d]pyrimidine, a quinazolinyl, repotrectinib, Ro 08-2750, a substituted pyrazolo[1,5a]pyrimidine, sitravatinib, SNA-125, tavilermide, thiazole 20h, ARRY-772, AZD7451, belizatinib, selitrectinib, crizotinib, ONO-7579, merestinib, ensartinib, TSR-011, MGCD516, altiratinib, cabozantinib, XL-184, DCC-2701, F17752, regorafenib, dovitinib, BMS-754807, ENMD-2076, BMS-777607, midostaurin, MK5108, PF-03814735, SNS-314, nintedanib, ponatinib, foretinib, AZD 1480, or VMD-928. In some embodiments, the kinase inhibitor is ARRY-470 or larotrectinib, AZ-23, danusertib (PHA-739358), entrectinib, lestaurtinib (CEP-701), AZD7451, belizatinib, selitrectinib, or crizotinib.

In one embodiment, the anti-cancer agent is a kinase inhibitor, e.g., a multi-kinase inhibitor. Exemplary multi-kinase inhibitors include, e.g., KRC-108, crizotinib, and K252a. In another embodiment, the NTRK kinase inhibitor is chosen from one or more of: AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), LOXO-195, a macrocyclic compound, ONO-5390556, oxindole 3, pegcantratinib (SNA-120), PHA-848125, PLX7486 (see e.g., Mok et al., 2016, CRI-CIMT-EATI-AACR Abstract A146, DOI: 10.1158/2326-6066.IMM2016-A146), a pyrazole derivative, a pyrazolo[1, 5a]pyrimidine, a pyridocarbazole, a pyridoquinazolinyl, a pyridotriazole, a pyrrolidinyl thiourea, a pyrrolidinyl urea, a pyrrolo[2, 3-d]pyrimidine, a quinazolinyl, repotrectinib, Ro 08-2750, a substituted pyrazolo[1,5a]pyrimidine, sitravatinib, SNA-125, tavilermide, thiazole 20h, ARRY-772, AZD7451, belizatinib, selitrectinib, crizotinib, ONO-7579 (see, e.g., clinical trial NCT03182257, available on the website https://clinicaltrials.gov/ct2/show/NCT03182257), merestinib (see, e.g., clinical trial NCT02920996, available at the website https://clinicaltrials.gov/ct2/show/NCT02920996), ensartinib (see, e.g., clinical trial NCT03574402, available at the website: https://clinicaltrials.gov/ct2/show/NCT03574402), TSR-011 (see, e.g., clinical trial NCT02048488, available at the website: https://clinicaltrials.gov/ct2/show/NCT02048488), MGCD516 (see, e.g., clinical trial NCT02219711, available at the website: https://clinicaltrials.gov/ct2/show/NCT02219711), altiratinib (see, e.g., clinical trial NCT02228811, available at the website: https://clinicaltrials.gov/ct2/show/NCT02228811), cabozantinib (see, e.g., clinical trial NCT01639508, available at the website: https://clinicaltrials.gov/ct2/show/NCT01639508), XL-184 (see, e.g., clinical trial NCT01639508, available at the website: https://clinicaltrials.gov/ct2/show/NCT01639508), DCC-2701 (see, e.g., clinical trial NCT02228811, available at the website: https://clinicaltrials.gov/ct2/show/NCT02228811), F17752 (see, e.g., Amatu et al., 2016; 27843590 and clinical trial EudraCT Number: 2013-003009-24), regorafenib (see, e.g., Khotskaya et al., 2017, 28174090, and the website: https://www.accessdata.fda.gov/drugsatfda_docs/label/2012/2030851b1.pdf), dovitinib (see, e.g., Sarker et al., 2008, 18381947), BMS-754807 (see, e.g., Carboni et al., 2009, 19996272), ENMD-2076 (see, e.g., Fletcher et al., 2011, 21177375), BMS-777607 (see, e.g., Schroeder et al., 2009, 19260711), midostaurin (see, e.g., Chi et al., 2012, 23,131,561; Okamura et al., 2018, 30637364), MK5108 (see, e.g., Shimomura et al., 2010, 20053775), PF-03814735 (see, e.g., Jani et al., 2010, 20354118), SNS-314 (see, e.g., Arbitrario et al., 2010, 19649632), nintedanib (see, e.g., Okamura et al., 2018; 30637364; Fuse et al., 2017; 28751539), ponatinib (see, e.g., Fuse et al., 2017; 28751539), foretinib (see, e.g., Nishiyama et al., 2018; 29463555), AZD 1480 (see, e.g., Gudernova et al., 2017; 29312610), or VMD-928. In another embodiment, the NTRK kinase inhibitor is chosen from one or more of: AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), LOXO-195, a macrocyclic compound, ONO-5390556, oxindole 3, pegcantratinib (SNA-120), PHA-848125, PLX7486, a pyrazole derivative, a pyrazolo[1, 5a]pyrimidine, a pyridocarbazole, a pyridoquinazolinyl, a pyridotriazole, a pyrrolidinyl thiourea, a pyrrolidinyl urea, a pyrrolo[2, 3-d]pyrimidine, a quinazolinyl, repotrectinib, Ro 08-2750, a substituted pyrazolo[1,5a]pyrimidine, sitravatinib, SNA-125, tavilermide, thiazole 20h, ARRY-772, AZD7451, belizatinib, selitrectinib, crizotinib, ONO-7579, merestinib, ensartinib, TSR-011, MGCD516, altiratinib, cabozantinib, XL-184, DCC-2701, F17752, regorafenib, dovitinib, BMS-754807, ENMD-2076, BMS-777607, midostaurin, MK5108, PF-03814735, SNS-314, nintedanib, ponatinib, foretinib, AZD 1480, and VMD-928.

In one embodiment, the kinase inhibitor is entrectinib (also known as RXDX-101 or NMS-E628). Entrectinib is a selective tyrosine kinase inhibitor, with inhibitory activity against TrkA, TrkB, and TrkC; C-ros oncogene 1 (ROS1); and anaplastic lymphoma kinase (ALK). Entrectinib is administered orally. Entrectinib has the chemical name: N-[5-(3,5-Difluorobenzyl)-1H-indazol-3-yl]-4-(4-methyl-1-piperazinyl)-2-(tetrahydro-2H-pyran-4-ylamino)benzamide. Entrectinib has the following structure:

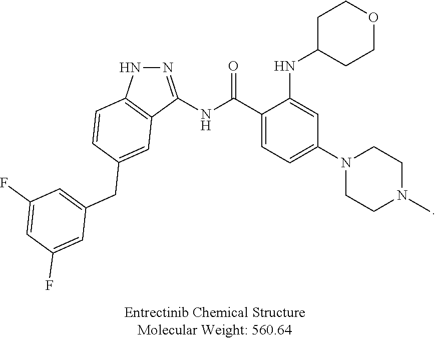 [SMILES: CN1CCN(c2ccc(C(=O)Nc3n[nH]c4ccc(Cc5cc(F)cc(F)c5)cc34)c(NC3CCOCC3)c2)CC1]

Clinical benefit with entrectinib monotherapy has been achieved for adult and pediatric patients with various solid tumors with and without CNS metastases and with NTRK fusions (Demetri et al., 2018; ESMO Abstract LBA17, Siena et al., 2019; ASCO Abstract 3017, Drilon et al., 2017; 28,183,697, Robinson et al., 2019; ASCO Abstract 10009, Doebele et al., 2019; ASCO Abstract 9070, Doebele et al., 2018; WCLC Abstract OA02.01), and preclinical sensitivity has been observed in NTRK fusion-positive AML cell lines (Smith et al., 2018; 29237803). In a Phase 1 trial, responses were restricted to patients harboring NTRK rearrangements (Drilon et al., 2017; 28183697).

Selitrectinib: In patients with NTRK fusion-positive cancers previously treated with at least 1 prior TRK inhibitor, treatment with selitrectinib achieved an ORR of 34% (10/29) with an ORR of 45% (9/20) in patients harboring a TRK kinase mutation (Hyman et al., 2019; AACR Abstract CT127).

In one embodiment, the kinase inhibitor is lestaurtinib (also known as CEP-701, rINN, KT 5555, SP 924). Lestaurtinib is an orally bioavailable indolocarbazole derivative with antineoplastic properties. Lestaurtinib is a tyrosine kinase inhibitor, with inhibitory activity against TrkA, TrkB, TrkC, FLT3, and JAK2. Lestaurtinib has the chemical name: (5S,6S,8R)-6-hydroxy-6-(hydroxymethyl)-5-methyl-7,8,14,15-tetrahydro-5H-16-oxa-4b,8a,14-triaza-5,8-methanodibenzo[b,h]cycloocta[jkl]cyclopenta[e]-as-indacen-13(6H)-one; and has the following structure:

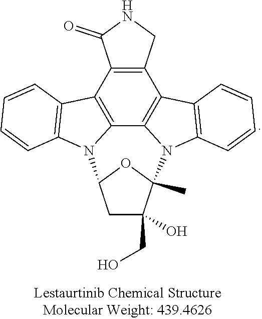 [SMILES: C[C@]12O[C@H](C[C@]1(O)CO)n1c3ccccc3c3c4c(c5c6ccccc6n2c5c31)CNC4=O]

In another embodiment, the inhibitor is AZ-23. AZ-23 is selective tyrosine kinase Trk inhibitor with IC50 of 2 and 8 nM for TrkA and TrkB, respectively. AZ-23 has the chemical name: 5-chloro-N-[(1S)-1-(5-fluoropyridin-2-yl)ethyl]-N′-(5-propan-2-yloxy-1H-pyrazol-3-yl)pyrimidine-2,4-diamine; and the chemical structure:

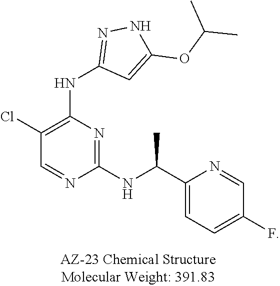 [SMILES: CC(C)Oc1cc(Nc2nc(N[C@@H](C)c3ccc(F)cn3)ncc2Cl)n[nH]1]

In another embodiment, the inhibitor is GW 441756. GW 441756 is a potent and orally active TrkA kinase inhibitor (IC50=2 nM) that displays more than 100-fold selectivity over a range of other kinases. GW 441756 has the chemical name: 3-[1-(1-Methyl-1H-indol-3-yl)-meth-(Z)-ylidene]-1,3-dihydro-pyrrolo[3,2-b]pyridin-2-one; and the chemical structure:

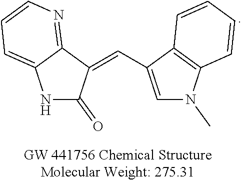 [SMILES: Cn1cc(/C=C2\C(=O)Nc3cccnc32)c2ccccc21]

In another embodiment, the inhibitor is isothiazole 5n. Isothiazole 5n is a TrkA kinase inhibitor with an IC50 of less than 1 nM. Isothiazole 5n has the chemical structure:

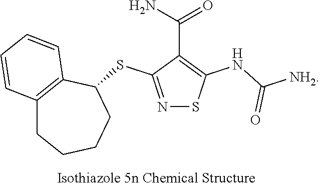 [SMILES: NC(=O)Nc1snc(S[C@@H]2CCCCc3ccccc32)c1C(N)=O]

In another embodiment, the kinase inhibitor is indenopyrrolocarboazole 12a. Indenopyrrolocarboazole 12a is a TrkA kinase inhibitor with an IC50 of 8 nM. Indenopyrrolocarboazole 12a has the following structure:

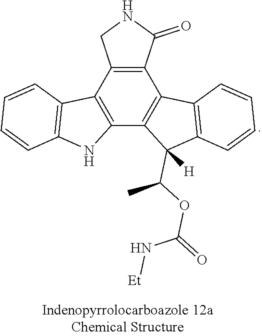 [SMILES: CCNC(=O)O[C@@H](C)[C@H]1c2ccccc2-c2c3c(c4c([nH]c5ccccc54)c21)CNC3=O]

In another embodiment, the kinase inhibitor is thiazole 20h. Thiazole 20h is a TrkA kinase inhibitor with an IC50 of 0.6 nM. Thiazole 20h has the following structure:

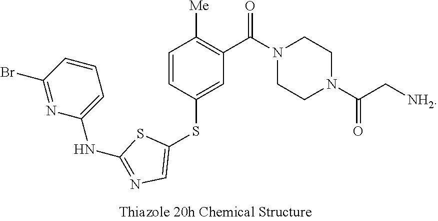 [SMILES: Cc1ccc(Sc2cnc(Nc3cccc(Br)n3)s2)cc1C(=O)N1CCN(C(=O)CN)CC1]

In another embodiment, the kinase inhibitor is oxindole 3. Oxindole 3 is a TrkA kinase inhibitor with an IC50 of 2 nM. Oxindole 3 has the following structure:

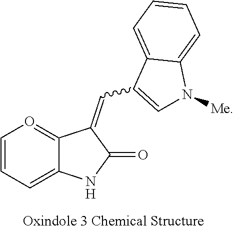

In another embodiment, the kinase inhibitor is pyridocarbazole. Pyridocarbazole is a TrkA kinase inhibitor with an IC50 of 6 nM. Pyridocarbazole has the following structure:

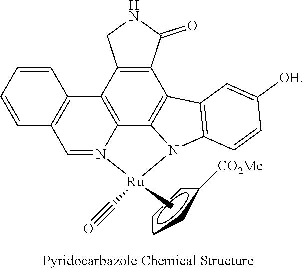

In another embodiment, the kinase inhibitor is AR523. AR523 is a pan-Trk inhibitor which demonstrates similar activity against TrkA, TrkB and TrkC receptors.

In another embodiment, the kinase inhibitor is K252a. K252a is a Trk inhibitor which inhibits tyrosine phosphorylation of Trk A. K252a has the chemical name: (9S-(9α,10β,12α))-2,3,9,10,11,12-hexahydro-10-hydroxy-10-(methoxycarbonyl)-9-methyl-9,12-epoxy-1H-diindolo[1,2,3-fg:3′,2′,1′-kl]pyrrolo[3,4-i][1,6]benzodiazocin-1-one; and has the following structure:

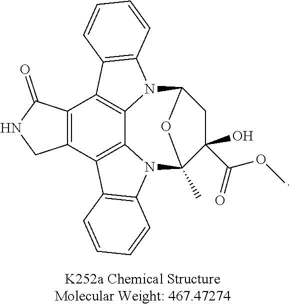 [SMILES: COC(=O)[C@@]1(O)C[C@H]2O[C@]1(C)n1c3ccccc3c3c4c(c5c6ccccc6n2c5c31)C(=O)NC4]

In another embodiment, the kinase inhibitor is GNF-5837. GNF-5837 is a potent pan-Trk inhibitor. GNF-5837 has the chemical name: N-[3-[[2,3-Dihydro-2-oxo-3-(1H-pyrrol-2-ylmethylene)-1H-indol-6-yl]amino]-4-methylphenyl]-N′-[2-fluoro-5-(trifluoromethyl)phenyl]urea; and has the following structure:

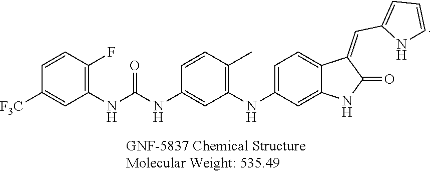 [SMILES: Cc1ccc(NC(=O)Nc2cc(C(F)(F)F)ccc2F)cc1Nc1ccc2c(c1)NC(=O)/C2=C\c1ccc[nH]1]

In another embodiment, the kinase inhibitor is AG 879 (Tyrphostin AG 879). AG 879 is an inhibitor of the tyrosine kinase activity of nerve growth factor (NGF) TrkA. AG 879 has the chemical name (2E)-3-[3,5-Bis(1,1-dimethylethyl)-4-hydroxyphenyl]-2-cyano-2-propenethioamide; and has the following structure:

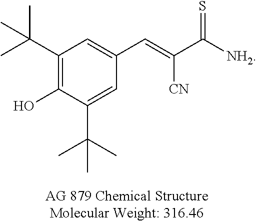 [SMILES: CC(C)(C)c1cc(/C=C(\C#N)C(N)=S)cc(C(C)(C)C)c1O]

In another embodiment, the kinase inhibitor is Ro 08-2750. Ro 08-2750 is a non-peptide inhibitor of NGF that binds the NGF dimer (K_{D}˜1 μM) possibly causing a conformational change. Ro 08-2750 has the following structure:

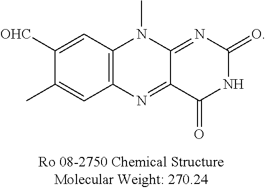 [SMILES: Cc1cc2nc3c(=O)[nH]c(=O)nc-3n(C)c2cc1C=O]

In another embodiment, the kinase inhibitor is AZ623. AZ623 is a novel potent and selective inhibitor of the Trk family of tyrosine kinases.

In another embodiment, the kinase inhibitor is larotrectinib (previously known as LOXO-101 or ARRY-470). Larotrectinib is a pan-Trk inhibitor which demonstrates with an IC50 of 9.5, 24, and 24 against TrkA, TrkB and TrkC, respectively. Larotrectinib has the following chemical name and chemical structure:

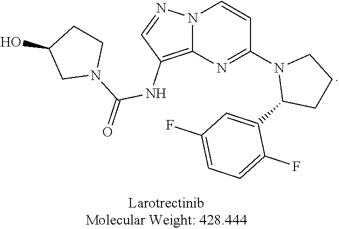 [SMILES: O=C(Nc1cnn2ccc(N3CCC[C@@H]3c3cc(F)ccc3F)nc12)N1CC[C@H](O)C1]

An analysis of combined data from a Phase 1, Phase ½, and Phase 2 trials reported an ORR of 81% (88/109) in adult and pediatric patients with various solid tumors, including soft tissue sarcoma, salivary gland tumor, thyroid carcinoma, GIST, lung tumor, melanoma, and CRC harboring NTRK fusions treated with larotrectinib; CR was observed in 17% of patients (Lassen et al., 2018; ESMO Abstract 4090). At 12 months of treatment, responses were ongoing in 75-81% of patients (Drilon et al., 2018; 29,466,156, Lassen et al., 2018; ESMO Abstract 4090). Acquired resistance to larotrectinib, putatively due to detected kinase domain mutations, was reported in 10 patients (Drilon et al., 2018; 29466156). The intracranial efficacy of larotrectinib has been demonstrated in several individuals with NTRK fusion-positive gliomas or brain metastases (Ziegler et al., 2018; 30220707, Schram et al., 2017; AACR abstract LB-302, Lassen et al., 2018; ESMO Abstract 4090).

In another embodiment, the kinase inhibitor is crizotinib. Durable clinical responses have also been reported in patients with NTRK1 fusion-positive tumors treated with the multi-kinase inhibitor crizotinib (Wong et al., 2015; 26563356, Mody et al., 2015; 26,325,560, Bender et al., 2019; 30709876, Vaishnavi et al., 2013; 24,162,815, Zhou et al., 2018; 30134855, Park et al., 2016; 26,716,414, Wang et al., 2019; 30691963).

In another embodiment, the kinase inhibitor is ARRY-772. ARRY-772 is a pan-Trk inhibitor which demonstrates with an IC50 of 10, 8.1, and 10 against TrkA, TrkB and TrkC, respectively.

In another embodiment, the kinase inhibitor is ARRY-772. ARRY-772 is a pan-Trk inhibitor which demonstrates with an IC50 of 2, 2.1, and 2.3 against TrkA, TrkB and TrkC, respectively.

In some embodiments, the anti-cancer therapy comprises a cellular therapy, and wherein the cellular therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the anti-cancer therapy comprises a nucleic acid that inhibits the expression of a fusion nucleic acid molecule of the present disclosure or the fusion polypeptide encoded by the fusion nucleic acid molecule. In some embodiments, the anti-cancer therapy comprises a nucleic acid that comprises a double-stranded RNA (dsRNA), a small interfering RNA (siRNA), or a small hairpin RNA (shRNA).

In some embodiments according to any of the embodiments described herein, the anti-cancer therapy or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. Further examples of anti-cancer therapies include, but are not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some embodiments, an anti-cancer therapy of the disclosure comprises a cyclin-dependent kinase (CDK) inhibitor, e.g., alone or in combination with an ALK-, NTRK1-, or NTRK3-targeted therapy. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with an ALK-, NTRK1-, or NTRK3-targeted therapy. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with an ALK-, NTRK1-, or NTRK3-targeted therapy) one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor, e.g., alone or in combination with an ALK-, NTRK1-, or NTRK3-targeted therapy. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with an ALK-, NTRK1-, or NTRK3-targeted therapy. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, RO4987655, RO5126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with an ALK-, NTRK1-, or NTRK3-targeted therapy. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with an ALK-, NTRK1-, or NTRK3-targeted therapy. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDCO0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with an ALK-, NTRK1-, or NTRK3-targeted therapy. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with an ALK-, NTRK1-, or NTRK3-targeted therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor, e.g., alone or in combination with an ALK-, NTRK1-, or NTRK3-targeted therapy.

In some embodiments, the anti-cancer therapy comprises a kinase inhibitor, e.g., alone or in combination with an ALK-, NTRK1-, or NTRK3-targeted therapy. In some embodiments, the kinase inhibitor is crizotinib, alectinib, ceritinib, lorlatinib, brigatinib, ensartinib (X-396), repotrectinib (TPX-0005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor, e.g., alone or in combination with an ALK-, NTRK1-, or NTRK3-targeted therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with an ALK-, NTRK1-, or NTRK3-targeted therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with an ALK-, NTRK1-, or NTRK3-targeted therapy. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPT0E028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor, e.g., alone or in combination with an ALK-, NTRK1-, or NTRK3-targeted therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin 03 inhibitor, e.g., alone or in combination with an ALK-, NTRK1-, or NTRK3-targeted therapy. In some embodiments, the integrin 03 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with an ALK-, NTRK1-, or NTRK3-targeted therapy. In some embodiments, the αIIbβ3 integrin inhibitor is abciximab, eptifibatide (Integrilin®), or tirofiban (Aggrastat®).

In some embodiments, the anti-cancer therapy comprises an mTOR inhibitor, e.g., alone or in combination with an ALK-, NTRK1-, or NTRK3-targeted therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with an ALK-, NTRK1-, or NTRK3-targeted therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with an ALK-, NTRK1-, or NTRK3-targeted therapy. In some embodiments, the MAPK inhibitor is SB203580, SKF-86002, BIRB-796, SC-409, RJW-67657, BIRB-796, VX-745, RO3201195, SB-242235, or MW181.

In some embodiments, the anti-cancer therapy comprises an EGFR inhibitor, e.g., alone or in combination with an ALK-, NTRK1-, or NTRK3-targeted therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., alone or in combination with an ALK-, NTRK1-, or NTRK3-targeted therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to a fusion nucleic acid molecule or polypeptide of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with an ALK-, NTRK1-, or NTRK3-targeted therapy. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen corresponding to a fusion nucleic acid molecule or polypeptide of the disclosure. In some embodiments, the cancer vaccine comprises DNA that encodes a neoantigen, e.g., a neoantigen corresponding to a fusion nucleic acid molecule or polypeptide of the disclosure. In some embodiments, the cancer vaccine comprises RNA that encodes a neoantigen, e.g., a neoantigen corresponding to a fusion nucleic acid molecule or polypeptide of the disclosure. In some embodiments, the cancer vaccine comprises a polynucleotide that encodes a neoantigen, e.g., a neoantigen corresponding to a fusion nucleic acid molecule or polypeptide of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a+-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to a fusion nucleic acid molecule or polypeptide of the disclosure

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a fusion nucleic acid molecule or polypeptide of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a fusion nucleic acid molecule or polypeptide of the disclosure, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen corresponding to a fusion nucleic acid molecule or polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor, e.g., alone or in combination with an ALK-, NTRK1-, or NTRK3-targeted therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1106 (nivolumab), MK-3475 (pembrolizumab, Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.570, MPDL3280A (atezolizumab), MDX-1105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (1B1310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MED14736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine, e.g., alone or in combination with an ALK-, NTRK1-, or NTRK3-targeted therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, I309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid that comprises or encodes a fusion nucleic acid molecule of the disclosure or a portion thereof, or a fusion polypeptide of the disclosure, or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes a fusion nucleic acid molecule of the disclosure or a portion thereof comprising a breakpoint. Methods for designing, optimizing, producing, and using dsRNAs, siRNAs, or shRNAs, are known in the art.

In some embodiments, the anti-cancer therapy comprises a chemotherapy, e.g., alone or in combination with an ALK-, NTRK1-, or NTRK3-targeted therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor, e.g., alone or in combination with an ALK-, NTRK1-, or NTRK3-targeted therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROS1, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU11248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent, e.g., alone or in combination with an ALK-, NTRK1-, or NTRK3-targeted therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy, e.g., alone or in combination with an ALK-, NTRK1-, or NTRK3-targeted therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer, e.g., alone or in combination with an ALK-, NTRK1-, or NTRK3-targeted therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent, e.g., alone or in combination with an ALK-, NTRK1-, or NTRK3-targeted therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent, e.g., alone or in combination with an ALK-, NTRK1-, or NTRK3-targeted therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent, e.g., alone or in combination with an ALK-, NTRK1-, or NTRK3-targeted therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent, e.g., alone or in combination with an ALK-, NTRK1-, or NTRK3-targeted therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 119, Marcel Dekker, 2002.

Formulations to be used for in vivo administration are sterile. This is readily accomplished by filtration through sterile filtration membranes or other methods known in the art.

In some embodiments, an anti-cancer therapy of the disclosure is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes.

In some embodiments, the individual has been previously treated, or is being treated, for cancer with a treatment for cancer, e.g., an anti-cancer therapy described herein or any other anti-cancer therapy or treatment known in the art. In some embodiments, the fusion nucleic acid molecule, and/or the fusion polypeptide, confers resistance of the cancer to the treatment for cancer.

In some embodiments, the methods further comprise detecting the presence or absence of a cancer in a sample from the individual. In some embodiments, the methods further comprise administering an effective amount of anti-cancer therapy to the individual, e.g., an anti-cancer therapy described herein.

In some embodiments of any of the methods provided herein, the sample is a sample described below. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the fusion nucleic acid molecule or polypeptide is detected in a tissue biopsy sample, in a liquid biopsy sample, or in both a tissue biopsy sample and a liquid biopsy sample, from the individual.

### Detection of Fusion Nucleic Acid Molecules and Polypeptides

Certain aspects of the present disclosure relate to detection of a fusion nucleic acid molecule of the disclosure, e.g., an ALK, NTRK1, or NTRK3 fusion nucleic acid molecule described herein, e.g., a patient sample. In some embodiments, the fusion nucleic acid molecule is detected in vitro.

Other aspects of the present disclosure relate to detection of a fusion polypeptide of the disclosure, e.g., a fusion polypeptide encoded by a fusion nucleic acid molecule of the disclosure, e.g., an ALK, NTRK1, or NTRK3 fusion nucleic acid molecule described herein, e.g., a patient sample. In some embodiments, the fusion polypeptide is detected in vitro.

Methods for detecting a fusion nucleic acid molecule of the disclosure, e.g., an ALK, NTRK1, or NTRK3 fusion nucleic acid molecule described herein, are known in the art. For example, in some embodiments, a fusion nucleic acid molecule is detected by sequencing part or all of a gene involved in the fusion nucleic acid molecule, e.g., an ALK, NTRK1, or NTRK3 gene, and/or a corresponding fusion partner gene described herein (e.g., as described in Table 1, and/or in the Examples herein), by next-generation or other sequencing of DNA, RNA, or cDNA. In some embodiments, a fusion nucleic acid molecule of the disclosure, e.g., an ALK, NTRK1, or NTRK3 fusion nucleic acid molecule described herein, is detected by PCR amplification of DNA, RNA, or cDNA. In some embodiments, a fusion nucleic acid molecule of the disclosure, e.g., an ALK, NTRK1, or NTRK3 fusion nucleic acid molecule described herein, is detected by in situ hybridization using one or more polynucleotides that hybridize to a locus involved in the fusion nucleic acid molecule, e.g., an ALK, NTRK1, or NTRK3 locus, and/or a corresponding fusion partner gene locus described herein (e.g., in Table 1, and/or in the Examples herein), e.g., using fluorescence in situ hybridization (FISH). In some embodiments, a fusion nucleic acid molecule of the disclosure, e.g., an ALK, NTRK1, or NTRK3 fusion nucleic acid molecule described herein, is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting a fusion nucleic acid molecule of the disclosure, e.g., an ALK, NTRK1, or NTRK3 fusion nucleic acid molecule described herein, are provided below.

In some embodiments, a fusion nucleic acid molecule of the disclosure, e.g., an ALK, NTRK1, or NTRK3 fusion nucleic acid molecule described herein, is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, a fusion nucleic acid molecule of the disclosure, e.g., an ALK, NTRK1, or NTRK3 fusion nucleic acid molecule described herein, is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, a fusion nucleic acid molecule of the disclosure, e.g., an ALK, NTRK1, or NTRK3 fusion nucleic acid molecule described herein, is detected using an in situ hybridization method, such as a fluorescence in situ hybridization (FISH) method.

In some embodiments, FISH analysis is used to identify the chromosomal rearrangement resulting in a fusion nucleic acid molecule as described herein. In some embodiments, FISH analysis is used to identify an RNA molecule comprising or encoding a fusion nucleic acid molecule of the disclosure, e.g., an ALK, NTRK1, or NTRK3 fusion nucleic acid molecule described herein. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

In some embodiments, a fusion nucleic acid molecule of the disclosure, e.g., an ALK, NTRK1, or NTRK3 fusion nucleic acid molecule described herein, is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, a fusion nucleic acid molecule of the disclosure, e.g., an ALK, NTRK1, or NTRK3 fusion nucleic acid molecule described herein, is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of a fusion nucleic acid molecule of the disclosure, e.g., an ALK, NTRK1, or NTRK3 fusion nucleic acid molecule described herein, in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, a fusion nucleic acid molecule of the disclosure, e.g., an ALK, NTRK1, or NTRK3 fusion nucleic acid molecule described herein, is detected using a sequencing method. Any method of sequencing known in the art can be used to detect a fusion nucleic acid molecule provided herein. Exemplary sequencing methods that may be used to detect a fusion nucleic acid molecule provided herein include those based on techniques developed by Maxam and Gilbert or Sanger. Automated sequencing procedures may also be used, e.g., including sequencing by mass spectrometry.

In some embodiments, a fusion nucleic acid molecule of the disclosure, e.g., an ALK, NTRK1, or NTRK3 fusion nucleic acid molecule described herein, is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, a fusion nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a fusion nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect a fusion nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual suspected of having or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual suspected of having or determined to have cancer), wherein the plurality of nucleic acid molecules comprises nucleic acid molecules corresponding to a fusion nucleic acid molecule of the disclosure, e.g., an ALK, NTRK1, or NTRK3 fusion nucleic acid molecule described herein (e.g., in Table 1, and/or in the Examples herein); (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the fusion nucleic acid molecule; (f) analyzing the plurality of sequence reads; and (g) based on the analysis, detecting the presence or absence of the fusion nucleic acid molecule in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to a fusion nucleic acid molecule of the disclosure (e.g., an ALK, NTRK1, or NTRK3 fusion nucleic acid molecule described herein (e.g., in Table 1, and/or in the Examples herein) in said library to produce an enriched sample; (e) sequencing the enriched sample, thereby producing a plurality of sequence reads; (f) analyzing the plurality of sequence reads for the presence of the fusion nucleic acid molecule; (g) detecting, based on the analyzing step, the presence or absence of the fusion nucleic acid molecule in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample, and the non-cancer nucleic acid molecules are derived from a non-tumor, cell-free DNA (cfDNA) fraction of the liquid biopsy sample.

In some embodiments of any of the methods, the one or more adapters comprise amplification primers, flow cell adaptor sequences, substrate adapter sequences, or sample index sequences. In some embodiments, the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to the fusion nucleic acid molecule of the disclosure (e.g., an ALK, NTRK1, or NTRK3 fusion nucleic acid molecule described herein (e.g., in Table 1, and/or in the Examples herein). In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the fusion nucleic acid molecule and producing nucleic acid hybrids; and (b) isolating the nucleic acid hybrids to produce the enriched sample. In some embodiments, the selectively enriching comprises amplifying the one or more nucleic acids comprising nucleotide sequences corresponding to the fusion nucleic acid molecule using a polymerase chain reaction (PCR) to produce an enriched sample. In some embodiments, the methods further comprise sequencing the enriched sample.

In some embodiments of any of the methods provided herein, the methods further comprise generating a genomic profile for the individual or the sample, based, at least in part, on detecting the presence or absence of the fusion nucleic acid molecule. In some embodiments, the genomic profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test.

In some embodiments of any of the methods provided herein, the methods further comprise selecting a treatment, administering a treatment, or applying a treatment to the individual based on the generated genomic profile, wherein the treatment comprises an anti-cancer therapy, e.g., as described herein.

In some embodiments of any of the methods provided herein, the methods further comprise generating a report indicating the presence or absence of the fusion nucleic acid molecule in the sample. In some embodiments, the methods further comprise generating, by the one or more processors, a report indicating the presence or absence of the fusion nucleic acid molecule in the sample. In some embodiments, the methods further comprise transmitting the report to a healthcare provider. In some embodiments, the report is transmitted via a computer network or a peer-to-peer connection.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample.

In some embodiments of any of the methods provided herein, the method further comprises determining the circulating tumor DNA (ctDNA) fraction of a liquid biopsy sample, e.g., as described herein in Example 1.

### Detection of Fusion Polypeptides

Also provided herein are methods of detecting a fusion polypeptide of the disclosure, or a fragment thereof, e.g., a fusion polypeptide encoded by a fusion nucleic acid molecule of the disclosure, e.g., an ALK, NTRK1, or NTRK3 fusion nucleic acid molecule described herein (e.g., as described in Table 1, and/or in the Examples herein), or a fragment thereof.

A fusion polypeptide provided herein, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a fusion polypeptide provided herein, or a fragment thereof, can be distinguished from a reference polypeptide, e.g., a non-mutant or wild type protein or polypeptide, with an antibody or antibody fragment that reacts differentially with a mutant protein or polypeptide (e.g., a fusion polypeptide provided herein or a fragment thereof) as compared to a reference protein or polypeptide. In some embodiments, a fusion polypeptide of the disclosure, or a fragment thereof, can be distinguished from a reference polypeptide, e.g., a non-mutant or wild type protein or polypeptide, by reaction with a detection reagent, e.g., a substrate, e.g., a substrate for catalytic activity, e.g., phosphorylation.

In some aspects, methods of detection of a fusion polypeptide of the disclosure (e.g., a fusion polypeptide encoded by a fusion nucleic acid molecule of the disclosure, e.g., an ALK, NTRK1, or NTRK3 fusion nucleic acid molecule described herein, e.g., as described in Table 1, and/or in the Examples herein), or a fragment thereof, are provided, comprising contacting a sample, e.g., a sample described herein, comprising a fusion polypeptide described herein, with a detection reagent provided herein (e.g., an antibody of the disclosure), and determining if the fusion polypeptide is present in the sample.

### Detection Reagents

In some aspects, provided herein are reagents for detecting a fusion nucleic acid molecule of the disclosure, e.g., an ALK, NTRK1, or NTRK3 fusion nucleic acid molecule described herein, or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises a fusion nucleic acid molecule described herein or a fragment or portion thereof.

In other aspects, provided herein are reagents for detecting a fusion polypeptide of the disclosure, or a fragment thereof, e.g., a fusion polypeptide encoded by a fusion nucleic acid molecule of the disclosure, e.g., an ALK, NTRK1, or NTRK3 fusion nucleic acid molecule described herein (e.g., as described in Table 1, and/or in the Examples herein), or a fragment thereof, e.g., according to the methods of detection provided herein. In some embodiments, a detection reagent provided herein comprises an antibody or antibody fragment that specifically binds to a fusion polypeptide of the disclosure, or to a fragment thereof

### Baits

In some embodiments, nucleic acids corresponding to a gene involved in a fusion nucleic acid molecule described herein, e.g., an ALK, NTRK1, or NTRK3 gene, and/or a corresponding gene fusion partner as described herein (e.g., in Table 1, and/or in the Examples herein), are captured (e.g., from amplified nucleic acids) by hybridization with a bait molecule. Provided herein are bait molecules suitable for the detection of a fusion nucleic acid molecule of the disclosure, e.g., an ALK, NTRK1, or NTRK3 fusion nucleic acid molecule described herein.

In some embodiments, a bait molecule comprises a capture nucleic acid molecule configured to hybridize to a target nucleic acid molecule comprising a fusion nucleic acid molecule of the disclosure, e.g., an ALK, NTRK1, or NTRK3 fusion nucleic acid molecule described herein, or a fragment or portion thereof. In some embodiments, the capture nucleic acid molecule is configured to hybridize to the ALK, NTRK1, or NTRK3 fusion nucleic acid molecule of the target nucleic acid molecule.

In some embodiments, the capture nucleic acid molecule is configured to hybridize to a fragment of a fusion nucleic acid molecule of the disclosure, e.g., an ALK, NTRK1, or NTRK3 fusion nucleic acid molecule described herein. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a breakpoint or fusion junction of a fusion nucleic acid molecule of the disclosure, e.g., an ALK, NTRK1, or NTRK3 fusion nucleic acid molecule described herein.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint of a fusion nucleic acid molecule of the disclosure, e.g., an ALK, NTRK1, or NTRK3 fusion nucleic acid molecule described herein, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of an ALK, NTRK1, or NTRK3 gene, or in a breakpoint joining the introns or exons of an ALK, NTRK1, or NTRK3 gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein, e.g., in Table 1, and/or in the Examples herein).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence corresponding to an intron or an exon of one gene of a fusion molecule described herein (e.g., an ALK, NTRK1, or NTRK3 gene), in an intron or an exon of the other gene of a fusion molecule described herein (e.g., a corresponding gene fusion partner as described herein, e.g., in Table 1, and/or in the Examples herein), and/or a breakpoint joining the introns and/or exons.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having a breakpoint of a fusion nucleic acid molecule described herein, e.g., as described in Table 1, from a reference nucleotide sequence, e.g., a nucleotide sequence not having the breakpoint.

In some embodiments, the bait hybridizes to a breakpoint of a fusion nucleic acid molecule described herein, e.g., as described in Table 1, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Probes

Also provided herein are probes, e.g., nucleic acid molecules, suitable for the detection of a fusion nucleic acid molecule of the disclosure, e.g., an ALK, NTRK1, or NTRK3 fusion nucleic acid molecule described herein (e.g., in Table 1, and/or in the Examples herein). In some embodiments, a probe provided herein comprises a nucleic acid sequence configured to hybridize to a target nucleic acid molecule that is or comprises a fusion nucleic acid molecule of the disclosure, e.g., an ALK, NTRK1, or NTRK3 fusion nucleic acid molecule described herein, or a fragment or portion thereof. In some embodiments, the probe comprises a nucleic acid sequence configured to hybridize to the fusion nucleic acid molecule of the disclosure, or the fragment or portion thereof, of the target nucleic acid molecule. In some embodiments, the probe comprises a nucleic acid sequence configured to hybridize to a fragment or portion of the fusion nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint of a fusion nucleic acid molecule of the disclosure, e.g., as described in Table 1, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a gene involved in a fusion nucleic acid molecule described herein, e.g., an ALK, NTRK1, or NTRK3 gene, or in a breakpoint joining the introns or exons of the gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides) to an intron or exon of another gene (e.g., a corresponding gene fusion partner as described herein, e.g., in Table 1, and/or in the Examples herein).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of fusion nucleic acid molecules provided herein. For example, a probe provided herein may be used for detecting a fusion nucleic acid molecule of the disclosure, e.g., an ALK, NTRK1, or NTRK3 fusion nucleic acid molecule described herein, in a sample, e.g., a sample obtained from an individual. In some embodiments, the probe may be used for identifying cells or tissues that express a fusion nucleic acid molecule of the disclosure, e.g., an ALK, NTRK1, or NTRK3 fusion nucleic acid molecule described herein, e.g., by measuring levels of the fusion nucleic acid molecule. In some embodiments, the probe may be used for detecting levels of a fusion nucleic acid molecule of the disclosure, e.g., an ALK, NTRK1, or NTRK3 fusion nucleic acid molecule described herein, e.g., mRNA levels, in a sample of cells from an individual.

In some embodiments, a probe provided herein specifically hybridizes to a nucleic acid molecule comprising a rearrangement (e.g., a deletion, inversion, insertion, duplication, or other rearrangement) resulting in a fusion nucleic acid molecule of the disclosure, e.g., an ALK, NTRK1, or NTRK3 fusion nucleic acid molecule described herein.

In some embodiments, a probe of the disclosure distinguishes a nucleic acid, e.g., a genomic or transcribed nucleic acid, e.g., a cDNA or RNA, having a breakpoint of a fusion nucleic acid molecule of the disclosure, e.g., as described in Table 1, from a reference nucleotide sequence, e.g., a nucleotide sequence not having the breakpoint.

Also provided herein are isolated pairs of allele-specific probes, wherein, for example, the first probe of the pair specifically hybridizes to a fusion nucleic acid molecule of the disclosure, e.g., an ALK, NTRK1, or NTRK3 fusion nucleic acid molecule described herein, and the second probe of the pair specifically hybridizes to a corresponding wild type sequence (e.g., a wild type ALK, NTRK1, or NTRK3 nucleic acid molecule; and/or a wild type nucleic acid molecule corresponding to a gene fusion partner as described herein, e.g., in Table 1, and/or in the Examples herein). Probe pairs can be designed and produced for any of the fusion nucleic acid molecules described herein and are useful in detecting a somatic mutation in a sample. In some embodiments, a first probe of a pair specifically hybridizes to a mutation (e.g., the breakpoint of an alteration, rearrangement, inversion, duplication, deletion, insertion or translocation resulting in a fusion nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that is or comprises a fusion nucleic acid molecule of the disclosure, e.g., an ALK, NTRK1, or NTRK3 fusion nucleic acid molecule described herein.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a fusion nucleic acid molecule of the disclosure, e.g., an ALK, NTRK1, or NTRK3 fusion nucleic acid molecule described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)-or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a breakpoint of a fusion nucleic acid molecule of the disclosure, e.g., an ALK, NTRK1, or NTRK3 fusion nucleic acid molecule described herein (e.g., as described in Table 1), and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises a fusion nucleic acid molecule of the disclosure, e.g., an ALK, NTRK1, or NTRK3 fusion nucleic acid molecule described herein (e.g., in Table 1, and/or in the Examples herein), or a fragment or portion thereof. In some embodiments, the oligonucleotide comprises a nucleotide sequence configured to hybridize to the fusion nucleic acid molecule of the target nucleic acid molecule. In some embodiments, the oligonucleotide comprises a nucleotide sequence configured to hybridize to a fragment or portion of the fusion nucleic acid molecule of the target nucleic acid molecule.

In some embodiments, the oligonucleotide, e.g., the primer, comprises a nucleotide sequence configured to hybridize to a breakpoint of a fusion nucleic acid molecule of the disclosure, e.g., an ALK, NTRK1, or NTRK3 fusion nucleic acid molecule described herein (e.g., as described in Table 1), and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of a gene involved in a fusion nucleic acid mole of the disclosure (e.g., an ALK, NTRK1, or NTRK3 gene), to a breakpoint of a fusion nucleic acid molecule described herein (e.g., as described in Table 1), and/or to an intron or exon of another gene (e.g., a corresponding gene fusion partner as described herein, e.g., in Table 1, and/or in the Examples herein).

In some embodiments, the oligonucleotide comprises a nucleotide sequence corresponding to a fusion nucleic acid molecule of the disclosure, e.g., an ALK, NTRK1, or NTRK3 fusion nucleic acid molecule described herein (e.g., in Table 1, and/or in the Examples herein). In some embodiments, the oligonucleotide comprises a nucleotide sequence corresponding to a fragment or a portion of the fusion nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a fusion nucleic acid molecule provided herein. In some embodiments, the oligonucleotide comprises a nucleotide sequence complementary to a fragment or a portion of the fusion nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises a fusion nucleic acid molecule of the disclosure, e.g., an ALK, NTRK1, or NTRK3 fusion nucleic acid molecule described herein (e.g., in Table 1, and/or in the Examples herein), or a fragment thereof, e.g., using PCR. In some embodiments, the oligonucleotide may be used to sequence a nucleic acid molecule that is or comprises a fusion nucleic acid molecule provided herein, or a fragment thereof. In some embodiments, the oligonucleotide may be used to amplify a nucleic acid molecule comprising a breakpoint of a fusion nucleic acid molecule described herein (e.g., as described in Table 1), e.g., using PCR. In some embodiments, the oligonucleotide may be used to sequence a nucleic acid molecule comprising a breakpoint of a fusion nucleic acid molecule described herein (e.g., as described in Table 1).

In some embodiments, pairs of oligonucleotides, e.g., pairs of primers, are provided herein, which are configured to hybridize to a nucleic acid molecule that is or comprises a fusion nucleic acid molecule of the disclosure, e.g., an ALK, NTRK1, or NTRK3 fusion nucleic acid molecule described herein (e.g., as described in Table 1, and/or in the Examples herein), or a fragment thereof. In some embodiments, a pair of oligonucleotides of the disclosure may be used for directing amplification of the fusion nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a breakpoint of a fusion nucleic acid molecule described herein (e.g., as described in Table 1), e.g., for use in directing amplification of the corresponding fusion nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a fusion nucleic acid molecule of the disclosure, e.g., an ALK, NTRK1, or NTRK3 fusion nucleic acid molecule described herein (e.g., as described in Table 1, and/or in the Examples herein), or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a breakpoint of a fusion nucleic acid molecule described herein (e.g., as described in Table 1), from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in a fusion nucleic acid molecule of the disclosure, e.g., an ALK, NTRK1, or NTRK3 fusion nucleic acid molecule described herein (e.g., as described in Table 1, and/or in the Examples herein). In another aspect, provided herein is a primer or primer set for amplifying a nucleic acid molecule comprising an alteration, rearrangement, chromosomal inversion, insertion, deletion, translocation, duplication or other rearrangement resulting in a fusion nucleic acid molecule of the disclosure, e.g., an ALK, NTRK1, or NTRK3 fusion nucleic acid molecule described herein (e.g., as described in Table 1, and/or in the Examples herein). In certain aspects, provided herein are allele-specific oligonucleotides, e.g., primers, wherein a first oligonucleotide of a pair specifically hybridizes to a mutation (e.g., a breakpoint of a fusion nucleic acid molecule described herein, e.g., as described in Table 1), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a breakpoint of a fusion nucleic acid molecule described herein, e.g., as described in Table 1), and a second oligonucleotide of the pair specifically hybridizes to a sequence downstream of the mutation.

In some embodiments, the oligonucleotide, e.g., the primer, hybridizes to a breakpoint of a fusion nucleic acid molecule described herein, e.g., as described in Table 1, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Antibodies

Provided herein are antibodies or antibody fragments that specifically bind to a fusion polypeptide of the disclosure, or a fragment thereof, e.g., a fusion polypeptide encoded by a fusion nucleic acid molecule of the disclosure, e.g., an ALK, NTRK1, or NTRK3 fusion nucleic acid molecule described herein (e.g., as described in Table 1, and/or in the Examples herein), or a fragment thereof.

The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity, e.g., binding to a fusion polypeptide of the disclosure, or a fragment thereof, e.g., a fusion polypeptide encoded by a fusion nucleic acid molecule of the disclosure, e.g., an ALK, NTRK1, or NTRK3 fusion nucleic acid molecule described herein (e.g., as described in Table 1, and/or in the Examples herein), or a fragment thereof.

In some embodiments, a fusion polypeptide of the disclosure, or a fragment thereof, e.g., a fusion polypeptide encoded by a fusion nucleic acid molecule of the disclosure, e.g., an ALK, NTRK1, or NTRK3 fusion nucleic acid molecule described herein (e.g., as described in Table 1, and/or in the Examples herein), or a fragment thereof, is used as an immunogen to generate one or more antibodies of the disclosure, e.g., using standard techniques for polyclonal and monoclonal antibody preparation. In some embodiments, a fusion polypeptide provided herein, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a fusion polypeptide of the disclosure, or a fragment thereof, e.g., a fusion polypeptide encoded by a fusion nucleic acid molecule of the disclosure, e.g., an ALK, NTRK1, or NTRK3 fusion nucleic acid molecule described herein (e.g., as described in Table 1, and/or in the Examples herein), or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on a fusion polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a fusion polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a fusion polypeptide provided herein, or a fragment thereof, by standard techniques, such as affinity chromatography or immunoprecipitation. In some embodiments, an antibody of the disclosure can be used to detect a fusion polypeptide provided herein, or a fragment thereof, e.g., in a tissue sample, cellular lysate, or cell supernatant, in order to evaluate the level and/or pattern of expression of the fusion polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a fusion polypeptide provided herein) in tissues or body fluids (e.g., in a tumor cell-containing tissue or body fluid), e.g., according to the methods provided herein.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸ μM or less, e.g., from 10⁻⁸ μM to 10⁻¹ μM, e.g., from 10⁻⁹ μM to 10⁻¹³ μM). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a fusion polypeptide provided herein). In some embodiments, a RIA is performed with the Fab version of an antibody of the disclosure and its antigen (e.g., a fusion polypeptide provided herein).

In certain embodiments, an antibody provided herein is an antibody fragment. Antibody fragments include, but are not limited to, Fab, Fab′, Fab′-SH, F(ab′)₂, Fv, and single-chain antibody molecule (e.g., scFv) fragments, and other fragments described herein or known in the art.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are known in the art and described herein.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to a fusion polypeptide of the disclosure, and the other can be to any other antigen. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to a fusion polypeptide of the disclosure as well as another, different antigen.

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) Clq binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### Samples

A variety of materials can be the source of, or serve as, samples for use in any of the methods of the disclosure, such as the methods for detection of a fusion nucleic acid molecule or a fusion polypeptide of the disclosure, or fragments thereof.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids (e.g., for use in any of the methods for detection of fusion nucleic acid molecules provided herein) or proteins (e.g., for use in any of the methods for detection of fusion polypeptides provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises a fusion nucleic acid molecule or a fusion polypeptide of the disclosure.

In some embodiments, the sample is a control sample or a reference sample, e.g., not containing a fusion nucleic acid molecule or a fusion polypeptide described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the fusion nucleic acid molecules or fusion polypeptides described herein. In other embodiments, the reference sample is from a non-tumor or cancer sample, e.g., a blood control, a normal adjacent tumor (NAT), or any other non-cancerous sample from the same or a different individual.

In some embodiments, a fusion nucleic acid molecule of the disclosure is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual. In some embodiments, the sample comprises cDNA derived from an mRNA sample or from a sample comprising mRNA. In some embodiments, a fusion nucleic acid molecule of the disclosure is detected in a sample comprising cell-free DNA (cfDNA), cell-free RNA, and/or circulating tumor DNA (ctDNA). In some embodiments, a fusion nucleic acid molecule of the disclosure is detected in a sample comprising cell-free DNA (cfDNA) and/or circulating tumor DNA (ctDNA). In some embodiments, a fusion nucleic acid molecule of the disclosure is detected in a sample comprising circulating tumor DNA (ctDNA).

### Reporting

In some embodiments, the methods provided herein comprise generating a report, and/or providing a report to party.

In some embodiments, a report according to the present disclosure comprises information about one or more of: a fusion nucleic acid molecule or polypeptide of the disclosure, e.g., an ALK, NTRK1, or NTRK3 fusion nucleic acid molecule described herein (e.g., in Table 1, and/or in the Examples herein), or a fusion polypeptide encoded by such a fusion nucleic acid molecule; a cancer of the disclosure, e.g., comprising a fusion nucleic acid molecule or polypeptide of the disclosure; or a treatment, a therapy, or one or more treatment options for an individual having a cancer, such as a cancer of the disclosure (e.g., comprising a fusion nucleic acid molecule or polypeptide described herein).

In some embodiments, a report according to the present disclosure comprises information about the presence or absence of a fusion nucleic acid molecule or polypeptide of the disclosure, e.g., an ALK, NTRK1, or NTRK3 fusion nucleic acid molecule described herein (e.g., in Table 1, and/or in the Examples herein), or a fusion polypeptide encoded by such a fusion nucleic acid molecule, in a sample obtained from an individual, such as an individual having a cancer, e.g., a cancer provided herein. In one embodiment, a report according to the present disclosure indicates that a fusion nucleic acid molecule or polypeptide of the disclosure is present in a sample obtained from the individual. In one embodiment, a report according to the present disclosure indicates that a fusion nucleic acid molecule or polypeptide of the disclosure is not present in a sample obtained from the individual. In one embodiment, a report according to the present disclosure indicates that a fusion nucleic acid molecule or polypeptide of the disclosure has been detected in a sample obtained from the individual. In one embodiment, a report according to the present disclosure indicates that a fusion nucleic acid molecule or polypeptide of the disclosure has not been detected in a sample obtained from the individual. In some embodiments, the report comprises an identifier for the individual from which the sample was obtained.

In some embodiments, the report includes information on the role of a fusion nucleic acid molecule or polypeptide of the disclosure, e.g., an ALK, NTRK1, or NTRK3 fusion nucleic acid molecule described herein (e.g., in Table 1, and/or in the Examples herein), or a fusion polypeptide encoded by such a fusion nucleic acid molecule, or its wild type counterparts, in disease, such as in cancer. Such information can include one or more of: information on prognosis of a cancer, such as a cancer provided herein, e.g., comprising a fusion nucleic acid molecule or polypeptide described herein; information on resistance of a cancer, such as a cancer provided herein, e.g., comprising a fusion nucleic acid molecule or polypeptide described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising a fusion nucleic acid molecule or polypeptide described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting a fusion nucleic acid molecule or polypeptide of the disclosure, e.g., an ALK, NTRK1, or NTRK3 fusion nucleic acid molecule described herein (e.g., in Table 1, and/or in the Examples herein), or a fusion polypeptide encoded by such a fusion nucleic acid molecule, in the sample, or acquiring knowledge of the presence of the fusion nucleic acid molecule or polypeptide of the disclosure in the sample; and generating a report. In some embodiments, a report generated according to the methods provided herein comprises one or more of: information about the presence or absence of a fusion nucleic acid molecule or polypeptide of the disclosure, e.g., an ALK, NTRK1, or NTRK3 fusion nucleic acid molecule described herein (e.g., in Table 1, and/or in the Examples herein), or a fusion polypeptide encoded by such a fusion nucleic acid molecule, in the sample; an identifier for the individual from which the sample was obtained; information on the role of the fusion nucleic acid molecule or polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising a fusion nucleic acid molecule or polypeptide of the disclosure), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a fusion nucleic acid molecule or polypeptide of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of the fusion nucleic acid molecule or polypeptide of the disclosure in a sample obtained from an individual (e.g., an individual having a cancer).

### Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

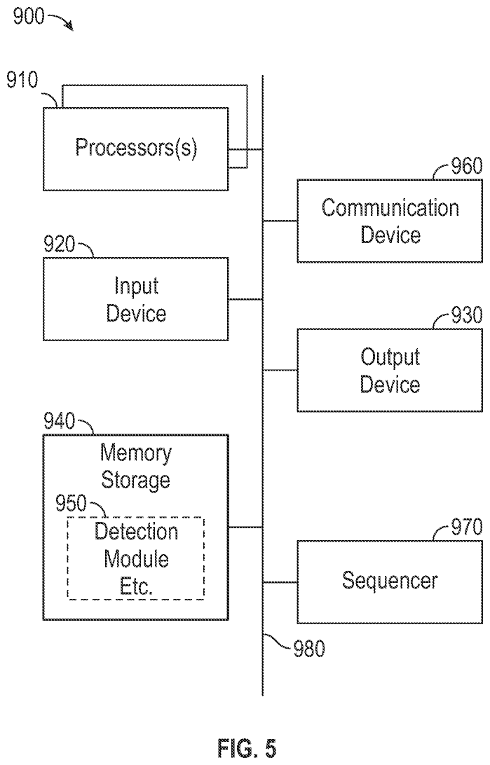

FIG. 5 illustrates an example of a computing device or system in accordance with one embodiment. Device 900 can be a host computer connected to a network. Device 900 can be a client computer or a server. As shown in FIG. 5, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting a fusion nucleic acid molecule of the disclosure. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting a fusion nucleic acid molecule of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

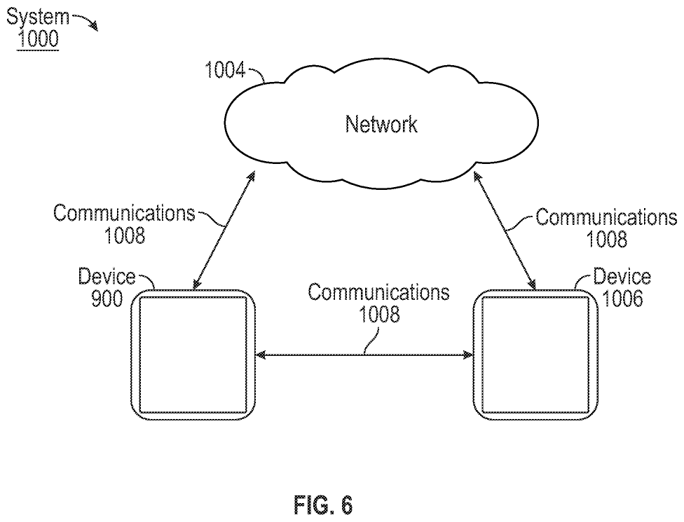

Device 900 may be connected to a network (e.g., network 1004, as shown in FIG. 6 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

FIG. 6 illustrates an example of a computing system in accordance with one embodiment. In computing system 1000, device 900 (e.g., as described above and illustrated in FIG. 5) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

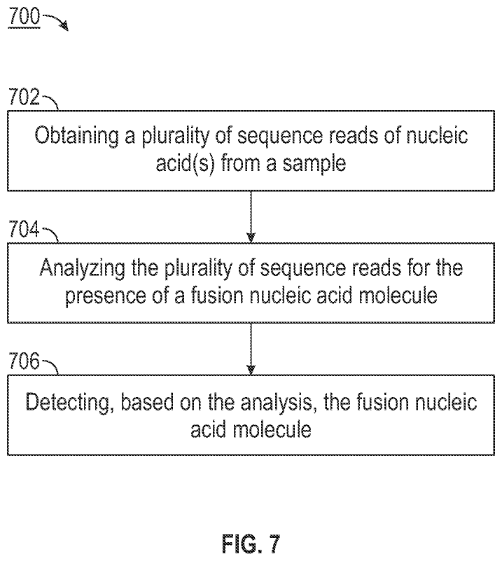

FIG. 7 illustrates an exemplary process 700 for detecting a fusion nucleic acid molecule of the disclosure in a sample, in accordance with some embodiments of the present disclosure. Process 700 is performed, for example, using one or more electronic devices implementing a software program. In some examples, process 700 is performed using a client-server system, and the blocks of process 700 are divided up in any manner between the server and a client device. In other examples, the blocks of process 700 are divided up between the server and multiple client devices. Thus, while portions of process 700 are described herein as being performed by particular devices of a client-server system, it will be appreciated that process 700 is not so limited. In some embodiments, the executed steps can be executed across many systems, e.g., in a cloud environment. In other examples, process 700 is performed using only a client device or only multiple client devices. In process 700, some blocks are, optionally, combined, the order of some blocks is, optionally, changed, and some blocks are, optionally, omitted. In some examples, additional steps may be performed in combination with the process 700. Accordingly, the operations as illustrated (and described in greater detail below) are exemplary by nature and, as such, should not be viewed as limiting.

At block 702, a plurality of sequence reads of one or more nucleic acid molecules is obtained, wherein the one or more nucleic acid molecules are derived from a sample obtained from an individual, e.g., as described herein. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: a fusion nucleic acid molecule of the disclosure, e.g., an ALK, NTRK1, or NTRK3 fusion nucleic acid molecule described herein (e.g., in Table 1, and/or in the Examples herein); or a gene involved in a fusion nucleic acid molecule of the disclosure; or fragments thereof. Optionally, prior to obtaining the sequence reads, the sample is purified, enriched (e.g., for nucleic acid(s) corresponding to: a fusion nucleic acid molecule of the disclosure, e.g., an ALK, NTRK1, or NTRK3 fusion nucleic acid molecule described herein (e.g., in Table 1, and/or in the Examples herein); or a gene involved in a fusion nucleic acid molecule of the disclosure; or fragments thereof), and/or subjected to PCR amplification. At block 704, an exemplary system (e.g., one or more electronic devices) analyzes the plurality of sequence reads for the presence of a fusion nucleic acid molecule of the disclosure, e.g., an ALK, NTRK1, or NTRK3 fusion nucleic acid molecule described herein (e.g., in Table 1, and/or in the Examples herein), or a fragment thereof. At block 706, the system detects (e.g., based on the analysis) a fusion nucleic acid molecule of the disclosure, e.g., an ALK, NTRK1, or NTRK3 fusion nucleic acid molecule described herein (e.g., in Table 1, and/or in the Examples herein), or a fragment thereof, in the sample.

In some embodiments, the fusion nucleic acid molecule is an ALK, NTRK1, or NTRK3 fusion nucleic acid molecule listed in Table 1. In some embodiments, the cancer is any of the cancers described herein, e.g., above. In some embodiments, the fusion nucleic acid molecule is an ALK, NTRK1, or NTRK3 fusion nucleic acid molecule listed in Table 1, and the cancer is the cancer corresponding to the ALK, NTRK1, or NTRK3 fusion nucleic acid molecule as listed in Table 1. In some embodiments, the fusion nucleic acid molecule is an ALK, NTRK1, or NTRK3 fusion nucleic acid molecule listed in Table 1, the cancer is the cancer corresponding to the ALK, NTRK1, or NTRK3 fusion nucleic acid molecule as listed in Table 1, and the fusion nucleic acid molecule comprises or results from a Breakpoint 1 and/or a Breakpoint 2 corresponding to the ALK, NTRK1, or NTRK3 fusion nucleic acid molecule as listed in Table 1.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the disclosed methods for determining the presence or absence of a fusion nucleic acid molecule of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of a fusion nucleic acid molecule of the disclosure as part of a genomic profiling process can improve the validity of, e.g., disease detection calls, made on the basis of the genomic profile by, for example, independently confirming the presence of the fusion nucleic acid molecule of the disclosure in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the individual may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

Accordingly, in some embodiments of any of the methods, systems, devices, non-transitory computer readable storage media, or processes of the disclosure, a genomic profile for the sample or for the individual is generated based at least in part on detecting a fusion nucleic acid molecule of the disclosure, or a fragment thereof, in the sample. In some embodiments, the individual is administered a treatment based at least in part on the genomic profile, e.g., a treatment described herein. In some embodiments, the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profile further comprises results from a nucleic acid sequencing-based test. In some embodiments, the genomic profile further comprises/indicates/comprises information on presence or absence of mutations in one or more additional genes, e.g., a panel of known oncogenes and/or tumor suppressors. In some embodiments, the genomic profile is obtained from a genomic profiling assay (such as a cancer- or tumor-related genomic profiling assay), e.g., as obtained using any of the sequencing methodologies described herein. In some embodiments, the genomic profile includes information from whole-genome or whole-exome sequencing. In some embodiments, the genomic profile includes information from targeted sequencing. In some embodiments, the genomic profile includes information from NGS. In some embodiments, the genomic profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known oncogenes and/or tumor suppressors.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting a fusion nucleic acid molecule of the disclosure in a sample, e.g., an ALK, NTRK1, or NTRK3 fusion nucleic acid molecule described herein and/or in Table 1, and/or in the Examples herein; or a fusion polypeptide encoded by a fusion nucleic acid molecule of the disclosure.

In some embodiments, the kits or articles of manufacture comprise one or more probes of the disclosure for detecting a fusion nucleic acid molecule of the disclosure in a sample, e.g., an ALK, NTRK1, or NTRK3 fusion nucleic acid molecule described herein and/or in Table 1, and/or in the Examples herein, e.g., according to any detection method known in the art or described herein. In some embodiments, the kits or articles of manufacture comprise one or more baits (e.g., one or more bait molecules) of the disclosure for detecting a fusion nucleic acid molecule of the disclosure in a sample, e.g., an ALK, NTRK1, or NTRK3 fusion nucleic acid molecule described herein and/or in Table 1, and/or in the Examples herein, e.g., according to any detection method known in the art or described herein. In some embodiments, the kits or articles of manufacture comprise one or more oligonucleotides (e.g., one or more primers) of the disclosure for detecting a fusion nucleic acid molecule of the disclosure in a sample, e.g., an ALK, NTRK1, or NTRK3 fusion nucleic acid molecule described herein and/or in Table 1, and/or in the Examples herein, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of a fusion nucleic acid molecule of the disclosure (e.g., a wild type ALK, NTRK1, or NTRK3 gene, and/or a wild type fusion partner gene described herein and/or in Table 1, and/or in the Examples herein). In some embodiments, one or more oligonucleotides, primers, probes or baits are capable of hybridizing to a fusion nucleic acid molecule of the disclosure, e.g., an ALK, NTRK1, or NTRK3 fusion nucleic acid molecule described herein and/or in Table 1, and/or in the Examples herein, or to a wild-type counterpart of the fusion nucleic acid molecule (e.g., a wild type ALK, NTRK1, or NTRK3 gene, and/or a wild type fusion partner gene described herein and/or in Table 1, and/or in the Examples herein). In some embodiments, the one or more oligonucleotides, primers, probes or baits of the present disclosure are capable of distinguishing a fusion nucleic acid molecule of the disclosure, e.g., an ALK, NTRK1, or NTRK3 fusion nucleic acid molecule described herein and/or in Table 1, and/or in the Examples herein, from a wild-type counterpart of the fusion nucleic acid molecule (e.g., a wild type ALK, NTRK1, or NTRK3 gene, and/or a wild type fusion partner gene described herein and/or in Table 1, and/or in the Examples herein). In some embodiments, the kit is for use according to any method of detecting fusion nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a fusion nucleic acid molecule of the disclosure, e.g., using one or more oligonucleotides, primers, probes or baits of the present disclosure.

In some embodiments, the kits or articles of manufacture comprise one or more antibodies or antibody fragments of the disclosure for detecting a fusion polypeptide encoded by a fusion nucleic acid molecule of the disclosure in a sample, e.g., an ALK, NTRK1, or NTRK3 fusion nucleic acid molecule described herein and/or in Table 1, and/or in the Examples herein, e.g., according to any detection method known in the art or described herein. In some embodiments, the kit or article of manufacture comprises a reagent (e.g., one or more antibodies of the present disclosure) for detecting the wild-type counterparts of a fusion polypeptide provided herein (e.g., a wild type ALK, NTRK1, or NTRK3 polypeptide, and/or a wild type polypeptide encoded by a fusion partner gene described herein and/or in Table 1, and/or in the Examples herein). In some embodiments, the kits or articles of manufacture comprise one or more antibodies of the present disclosure capable of binding to a fusion polypeptide provided herein, or to wild-type counterparts of the fusion polypeptide provided herein (e.g., a wild type ALK, NTRK1, or NTRK3 polypeptide, and/or a wild type polypeptide encoded by a fusion partner gene described herein and/or in Table 1, and/or in the Examples herein). In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a fusion polypeptide of the disclosure, e.g., using one or more antibodies of the present disclosure.

Further provided herein are kits or articles of manufacture comprising an anti-cancer therapy, such as an anti-cancer therapy described herein, and a package insert comprising instructions for using the anti-cancer therapy in a method of treating or delaying progression of cancer, e.g., by administration to an individual from whom a sample comprising a fusion nucleic acid molecule or polypeptide of the disclosure has been obtained. In some embodiments, the anti-cancer therapy is any of the anti-cancer therapies described herein for use in any of the methods for treating or delaying progression of cancer of the disclosure.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting a fusion nucleic acid molecule or polypeptide of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting a fusion nucleic acid molecule or polypeptide of the disclosure, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Expression Vectors, Host Cells and Recombinant Cells

Provided herein are vectors comprising or encoding a fusion nucleic acid molecule of the disclosure, e.g., an ALK, NTRK1, or NTRK3 fusion nucleic acid molecule described herein and/or in Table 1 and/or in the Examples herein, or a bait, a probe, or an oligonucleotide described herein, or fragments thereof.

In some embodiments, a vector provided herein comprises or encodes a fusion nucleic acid molecule of the disclosure, e.g., an ALK, NTRK1, or NTRK3 fusion nucleic acid molecule described herein and/or in Table 1, and/or in the Examples herein, or a nucleic acid molecule encoding a fusion polypeptide described herein.

In some embodiments, a vector provided herein is a nucleic acid molecule capable of transporting another nucleic acid to which it has been linked (e.g., fusion nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising fusion nucleic acid molecules, baits, probes, or oligonucleotides described herein, or fragments thereof) are also contemplated herein, including, e.g., replication defective retroviruses, adenoviruses and adeno-associated viruses.

In some embodiments, a vector provided herein comprises a fusion nucleic acid molecule, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., fusion nucleic acid molecules, baits, probes, or oligonucleotides described herein, or fragments thereof). In some embodiments, a regulatory sequence directs tissue-specific expression of a nucleotide sequence (e.g., fusion nucleic acid molecules, baits, probes, or oligonucleotides described herein, or fragments thereof). In some embodiments, a regulatory sequence directs inducible expression of a nucleotide sequence (e.g., fusion nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes a fusion nucleic acid molecule, a bait, a probe, or an oligonucleotide of the disclosure in the sense or the anti-sense orientation. In some embodiments, a vector (e.g., an expression vector) provided herein is introduced into host cells to thereby produce a polypeptide, e.g., a fusion polypeptide described herein, or a fragment or mutant form thereof.

In some embodiments, the design of a vector provided herein depends on such factors as the choice of the host cell to be transformed, the level of expression desired, and the like. In some embodiments, expression vectors are designed for the expression of the fusion nucleic acid molecules, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes a fusion nucleic acid molecule described herein, wherein the nucleotide sequence of the fusion nucleic acid molecule described herein has been altered (e.g., codon optimized) so that the individual codons for each encoded amino acid are those preferentially utilized in the host cell.

Also provided herein are host cells, e.g., comprising fusion nucleic acid molecules, fusion polypeptides, baits, probes, vectors, or oligonucleotides of the disclosure. In some embodiments, a host cell (e.g., a recombinant host cell or recombinant cell) comprises a vector described herein (e.g., an expression vector described herein). In some embodiments, a fusion nucleic acid molecule, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

Fusion nucleic acid molecules, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a fusion polypeptide of the disclosure, e.g., by culturing a host cell described herein (e.g., into which a recombinant expression vector encoding a polypeptide has been introduced) in a suitable medium such that the fusion polypeptide is produced. In another embodiment, the method further includes isolating a fusion polypeptide from the medium or the host cell.

### VI. Exemplary Embodiments

The following exemplary embodiments are representative of some aspects of the invention:

Embodiment 1. A method of identifying an individual having a cancer who may benefit from a treatment comprising an anti-cancer therapy, the method comprising detecting in a sample from the individual a fusion nucleic acid molecule, or a fusion polypeptide encoded by the fusion nucleic acid molecule; wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule;
   wherein detection of the fusion nucleic acid molecule or the fusion polypeptide encoded by the fusion nucleic acid molecule in the sample identifies the individual as one who may benefit from the treatment comprising the anti-cancer therapy.

Embodiment 2. A method of selecting a treatment for an individual having a cancer, the method comprising detecting in a sample from the individual a fusion nucleic acid molecule, or a fusion polypeptide encoded by the fusion nucleic acid molecule; wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule;
   wherein detection of the fusion nucleic acid molecule or the fusion polypeptide encoded by the fusion nucleic acid molecule in the sample identifies the individual as one who may benefit from the treatment comprising an anti-cancer therapy.

Embodiment 3. A method of identifying one or more treatment options for an individual having a cancer, the method comprising:
detecting in a sample from the individual a fusion nucleic acid molecule, or a fusion polypeptide encoded by the fusion nucleic acid molecule; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
generating a report comprising one or more treatment options identified for the individual based at least in part on detection of the fusion nucleic acid molecule or the fusion polypeptide encoded by the fusion nucleic acid molecule, wherein the one or more treatment options comprise an anti-cancer therapy.

Embodiment 4. A method of identifying one or more treatment options for an individual having a cancer, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
generating a report comprising one or more treatment options identified for the individual based at least in part on said knowledge, wherein the one or more treatment options comprise an anti-cancer therapy.

Embodiment 5. A method of selecting a treatment for an individual having cancer, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

Embodiment 6. A method of predicting survival of an individual having a cancer, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
wherein responsive to the acquisition of said knowledge: the individual is predicted to have longer survival when treated with a treatment comprising an anti-cancer therapy, as compared to survival of an individual whose cancer does not comprise the fusion nucleic acid molecule or the fusion polypeptide encoded by the fusion nucleic acid molecule.

Embodiment 7. A method of predicting survival of an individual having a cancer treated with a treatment comprising an anti-cancer therapy, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
      wherein responsive to the acquisition of said knowledge: the individual is predicted to have longer survival when treated with a treatment comprising an anti-cancer therapy, as compared to survival of an individual whose cancer does not exhibit the fusion nucleic acid molecule or the fusion polypeptide encoded by the fusion nucleic acid molecule.

Embodiment 8. A method of treating or delaying progression of cancer, the method comprising:
acquiring knowledge of a fusion nucleic acid molecule, or a fusion polypeptide encoded by the fusion nucleic acid molecule, in a sample from an individual; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
      responsive to said knowledge, administering to the individual an effective amount of a treatment that comprises an anti-cancer therapy.

Embodiment 9. A method of treating or delaying progression of cancer, comprising administering to an individual having a cancer an effective amount of a treatment that comprises an anti-cancer therapy, wherein the treatment is administered responsive to acquiring knowledge of a fusion nucleic acid molecule, or a fusion polypeptide encoded by the fusion nucleic acid molecule, in a sample from the individual; wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 10. A method of monitoring, evaluating or screening an individual having a cancer, comprising:
acquiring knowledge of a fusion nucleic acid molecule, or a fusion polypeptide encoded by the fusion nucleic acid molecule, in a sample from the individual; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule;
wherein responsive to the acquisition of said knowledge, the individual is predicted to have an improved response to treatment with an anti-cancer therapy, as compared to an individual whose cancer does not comprise the fusion nucleic acid molecule or the fusion polypeptide encoded by the fusion nucleic acid molecule.

Embodiment 11. A method of assessing a fusion nucleic acid molecule, or a fusion polypeptide encoded by the fusion nucleic acid molecule, in a cancer in an individual, comprising:
detecting in a sample from the individual a fusion nucleic acid molecule, or a fusion polypeptide encoded by the fusion nucleic acid molecule; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
providing an assessment of the fusion nucleic acid molecule or the fusion polypeptide encoded by the fusion nucleic acid molecule.

Embodiment 12. A method of detecting a fusion nucleic acid molecule, or a fusion polypeptide encoded by the fusion nucleic acid molecule, comprising: detecting in a sample from an individual having a cancer a fusion nucleic acid molecule, or a fusion polypeptide encoded by the fusion nucleic acid molecule; wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 13. A method of identifying an individual having a cancer who may benefit from a treatment comprising an anti-cancer therapy, the method comprising detecting in a sample from the individual a fusion nucleic acid molecule, or a fusion polypeptide encoded by the fusion nucleic acid molecule; wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule;
   wherein detection of the fusion nucleic acid molecule or the fusion polypeptide encoded by the fusion nucleic acid molecule in the sample identifies the individual as one who may benefit from the treatment comprising the anti-cancer therapy.

Embodiment 14. A method of selecting a treatment for an individual having a cancer, the method comprising detecting in a sample from the individual a fusion nucleic acid molecule, or a fusion polypeptide encoded by the fusion nucleic acid molecule; wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule;
   wherein detection of the fusion nucleic acid molecule or the fusion polypeptide encoded by the fusion nucleic acid molecule in the sample identifies the individual as one who may benefit from the treatment comprising an anti-cancer therapy.

Embodiment 15. A method of identifying one or more treatment options for an individual having a cancer, the method comprising:
detecting in a sample from the individual a fusion nucleic acid molecule, or a fusion polypeptide encoded by the fusion nucleic acid molecule; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
generating a report comprising one or more treatment options identified for the individual based at least in part on detection of the fusion nucleic acid molecule or the fusion polypeptide encoded by the fusion nucleic acid molecule, wherein the one or more treatment options comprise an anti-cancer therapy.

Embodiment 16. A method of identifying one or more treatment options for an individual having a cancer, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
generating a report comprising one or more treatment options identified for the individual based at least in part on said knowledge, wherein the one or more treatment options comprise an anti-cancer therapy.

Embodiment 17. A method of selecting a treatment for an individual having cancer, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
      wherein responsive to the acquisition of said knowledge: (i) the individual is classified as a candidate to receive a treatment comprising an anti-cancer therapy; and/or (ii) the individual is identified as likely to respond to a treatment that comprises an anti-cancer therapy.

Embodiment 18. A method of predicting survival of an individual having a cancer, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
      wherein responsive to the acquisition of said knowledge: the individual is predicted to have longer survival when treated with a treatment comprising an anti-cancer therapy, as compared to survival of an individual whose cancer does not comprise the fusion nucleic acid molecule or the fusion polypeptide encoded by the fusion nucleic acid molecule.

Embodiment 19. A method of predicting survival of an individual having a cancer treated with a treatment comprising an anti-cancer therapy, the method comprising:
acquiring knowledge of a fusion nucleic acid molecule, or a fusion polypeptide encoded by the fusion nucleic acid molecule, in a sample from the individual; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
      wherein responsive to the acquisition of said knowledge: the individual is predicted to have longer survival when treated with a treatment comprising an anti-cancer therapy, as compared to survival of an individual whose cancer does not exhibit the fusion nucleic acid molecule or the fusion polypeptide encoded by the fusion nucleic acid molecule.

Embodiment 20. A method of treating or delaying progression of cancer, the method comprising:
acquiring knowledge of a fusion nucleic acid molecule, or a fusion polypeptide encoded by the fusion nucleic acid molecule, in a sample from an individual; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
responsive to said knowledge, administering to the individual an effective amount of a treatment that comprises an anti-cancer therapy.

Embodiment 21. A method of treating or delaying progression of cancer, comprising administering to an individual having a cancer an effective amount of a treatment that comprises an anti-cancer therapy, wherein the treatment is administered responsive to acquiring knowledge of a fusion nucleic acid molecule, or a fusion polypeptide encoded by the fusion nucleic acid molecule, in a sample from the individual; wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 22. A method of monitoring, evaluating or screening an individual having a cancer, comprising:
acquiring knowledge of a fusion nucleic acid molecule, or a fusion polypeptide encoded by the fusion nucleic acid molecule, in a sample from the individual; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule;
      wherein responsive to the acquisition of said knowledge, the individual is predicted to have an improved response to treatment with an anti-cancer therapy, as compared to an individual whose cancer does not comprise the fusion nucleic acid molecule or the fusion polypeptide encoded by the fusion nucleic acid molecule.

Embodiment 23. A method of assessing a fusion nucleic acid molecule, or a fusion polypeptide encoded by the fusion nucleic acid molecule, in a cancer in an individual, comprising:
detecting in a sample from the individual a fusion nucleic acid molecule, or a fusion polypeptide encoded by the fusion nucleic acid molecule; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
providing an assessment of the fusion nucleic acid molecule or the fusion polypeptide encoded by the fusion nucleic acid molecule.

Embodiment 24. A method of detecting a fusion nucleic acid molecule, or a fusion polypeptide encoded by the fusion nucleic acid molecule, comprising: detecting in a sample from an individual having a cancer a fusion nucleic acid molecule, or a fusion polypeptide encoded by the fusion nucleic acid molecule; wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 25. A method of detecting the presence or absence of a cancer in an individual, the method comprising:
detecting the presence or absence of a cancer in a sample from the individual; and
detecting, in a sample from the individual, the presence or absence of a fusion nucleic acid molecule, or a fusion polypeptide encoded by the fusion nucleic acid molecule; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule.

Embodiment 26. A method of detecting the presence or absence of a cancer in an individual, the method comprising:
detecting the presence or absence of a cancer in a sample from the individual; and
detecting, in a sample from the individual, the presence or absence of a fusion nucleic acid molecule, or a fusion polypeptide encoded by the fusion nucleic acid molecule; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 27. The method of embodiment 25 or embodiment 26, comprising detecting the presence of the cancer in the sample.

Embodiment 28. The method of any one of embodiments 25-27, comprising detecting the presence of the fusion nucleic acid molecule or the fusion polypeptide encoded by the fusion nucleic acid molecule in the sample from the individual.

Embodiment 29. A method for monitoring progression or recurrence of a cancer in an individual, the method comprising:
detecting, in a first sample obtained from the individual at a first time point, the presence or absence of a fusion nucleic acid molecule, or a fusion polypeptide encoded by the fusion nucleic acid molecule;
detecting, in a second sample obtained from the individual at a second time point after the first time point, the presence or absence of a fusion nucleic acid molecule, or a fusion polypeptide encoded by the fusion nucleic acid molecule; and
providing an assessment of cancer progression or cancer recurrence in the individual based, at least in part, on the presence or absence of the fusion nucleic acid molecule or the fusion polypeptide encoded by the fusion nucleic acid molecule in the first sample and/or in the second sample;
wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule.

Embodiment 30. A method for monitoring progression or recurrence of a cancer in an individual, the method comprising:
detecting, in a first sample obtained from the individual at a first time point, the presence or absence of a fusion nucleic acid molecule, or a fusion polypeptide encoded by the fusion nucleic acid molecule;
detecting, in a second sample obtained from the individual at a second time point after the first time point, the presence or absence of a fusion nucleic acid molecule, or a fusion polypeptide encoded by the fusion nucleic acid molecule; and
providing an assessment of cancer progression or cancer recurrence in the individual based, at least in part, on the presence or absence of the fusion nucleic acid molecule or the fusion polypeptide encoded by the fusion nucleic acid molecule in the first sample and/or in the second sample;
wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 31. The method of embodiment 29 or embodiment 30, wherein the presence of the fusion nucleic acid molecule or the fusion polypeptide encoded by the fusion nucleic acid molecule in the first sample and/or in the second sample identifies the individual as having decreased risk of cancer progression or cancer recurrence when treated with a treatment comprising an anti-cancer therapy.

Embodiment 32. The method of any one of embodiments 29-31, further comprising selecting a treatment, administering a treatment, adjusting a treatment, adjusting a dose of a treatment, or applying a treatment to the individual based, at least in part, on detecting the presence of the fusion nucleic acid molecule or the fusion polypeptide encoded by the fusion nucleic acid molecule in the first sample and/or in the second sample, wherein the treatment comprises an anti-cancer therapy.

Embodiment 33. A method of detecting a fusion nucleic acid molecule, the method comprising:
providing a plurality of nucleic acid molecules obtained from a sample from an individual having a cancer, wherein the plurality of nucleic acid molecules comprises nucleic acid molecules corresponding to a fusion nucleic acid molecule, wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule;
optionally, ligating one or more adapters onto one or more nucleic acid molecules from the plurality of nucleic acid molecules;
optionally, amplifying the one or more ligated nucleic acid molecules from the plurality of nucleic acid molecules;
optionally, capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequence reads correspond to the fusion nucleic acid molecule;
analyzing the plurality of sequence reads; and
based on the analysis, detecting the presence or absence of the fusion nucleic acid molecule in the sample.

Embodiment 34. A method of detecting a fusion nucleic acid molecule, the method comprising:
providing a plurality of nucleic acid molecules obtained from a sample from an individual having a cancer, wherein the plurality of nucleic acid molecules comprises nucleic acid molecules corresponding to a fusion nucleic acid molecule, wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule;
optionally, ligating one or more adapters onto one or more nucleic acid molecules from the plurality of nucleic acid molecules;
optionally, amplifying the one or more ligated nucleic acid molecules from the plurality of nucleic acid molecules;
optionally, capturing amplified nucleic acid molecules from the amplified nucleic acid molecules;
sequencing, by a sequencer, the captured nucleic acid molecules to obtain a plurality of sequence reads that represent the captured nucleic acid molecules, wherein one or more of the plurality of sequence reads correspond to the fusion nucleic acid molecule;
analyzing the plurality of sequence reads; and
based on the analysis, detecting the presence or absence of the fusion nucleic acid molecule in the sample.

Embodiment 35. The method of embodiment 33 or embodiment 34, further comprising receiving, at one or more processors, sequence read data for the plurality of sequence reads.

Embodiment 36. The method of embodiment 35, wherein analyzing the plurality of sequence reads comprises identifying, using the one or more processors, the presence or absence of sequence reads corresponding to the fusion nucleic acid molecule.

Embodiment 37. The method of any one of embodiments 33-36, wherein the amplified nucleic acid molecules are captured by hybridization with one or more bait molecules.

Embodiment 38. A method of detecting a fusion nucleic acid molecule, the method comprising:
providing a sample from an individual having a cancer, wherein the sample comprises a plurality of nucleic acid molecules;
preparing a nucleic acid sequencing library from the plurality of nucleic acid molecules in the sample;
amplifying said library;
   selectively enriching for one or more nucleic acid molecules in said library that comprise nucleotide sequences corresponding to a fusion nucleic acid molecule to produce an enriched sample, wherein the fusion nucleic acid molecule is:
      (a) a neuropilin 2 (NRP2)-anaplastic lymphoma kinase (ALK) fusion nucleic acid molecule;
      (b) a phosphodiesterase 3A (PDE3A)-anaplastic lymphoma kinase (ALK) fusion nucleic acid molecule;
      (c) a proteasome 26S subunit, non-ATPase 14 (PSMD14)-anaplastic lymphoma kinase (ALK) fusion nucleic acid molecule;
      (d) an SFT2 domain containing 1 (SFT2D1)-anaplastic lymphoma kinase (ALK) fusion nucleic acid molecule;
      (e) a solute carrier family 37 member 3 (SLC37A3)-anaplastic lymphoma kinase (ALK) fusion nucleic acid molecule;
      (f) a transport and golgi organization 6 homolog (TANGO6)-anaplastic lymphoma kinase (ALK) fusion nucleic acid molecule; or
      (g) a WD repeat-containing protein 92 (WDR92)-anaplastic lymphoma kinase (ALK) fusion nucleic acid molecule;
   sequencing the enriched sample, thereby producing a plurality of sequence reads;
   analyzing the plurality of sequence reads for the presence of the fusion nucleic acid molecule; and
   detecting, based on the analyzing step, the presence or absence of the fusion nucleic acid molecule in the sample from the individual.

Embodiment 39. A method of detecting a fusion nucleic acid molecule, the method comprising:
providing a sample from an individual having a cancer, wherein the sample comprises a plurality of nucleic acid molecules;
preparing a nucleic acid sequencing library from the plurality of nucleic acid molecules in the sample;
amplifying said library;
   selectively enriching for one or more nucleic acid molecules in said library that comprise nucleotide sequences corresponding to a fusion nucleic acid molecule to produce an enriched sample, wherein the fusion nucleic acid molecule is:
      (a) a glycoprotein A33 (GPA33)-neurotrophic receptor tyrosine kinase 1 (NTRK1) fusion nucleic acid molecule;
      (b) a family with sequence similarity 19 (chemokine (C-C motif)-like, member A2) (FAM19A2)-neurotrophic receptor tyrosine kinase 1 (NTRK1) fusion nucleic acid molecule;
      (c) a cleavage and polyadenylation specific factor 6 (CPSF6)-neurotrophic receptor tyrosine kinase 1 (NTRK1) fusion nucleic acid molecule;
      (d) a SUN domain containing ossification factor (SUCO)-neurotrophic receptor tyrosine kinase 1 (NTRK1) fusion nucleic acid molecule;
      (e) a calcyclin binding protein (CACYBP)-neurotrophic receptor tyrosine kinase 1 (NTRK1) fusion nucleic acid molecule;
      (f) a zinc finger protein 382 (ZNF382)-neurotrophic receptor tyrosine kinase 1 (NTRK1) fusion nucleic acid molecule;
      (g) a nudE neurodevelopment protein 1 (NDE1)-neurotrophic receptor tyrosine kinase 3 (NTRK3) fusion nucleic acid molecule;
      (h) a DiGeorge syndrome critical region gene 5 (DGCR5)-neurotrophic receptor tyrosine kinase 3 (NTRK3) fusion nucleic acid molecule; or
      (i) a ubiquitin conjugating enzyme E2 Q2 pseudogene 1 (UBE2Q2P1)-neurotrophic receptor tyrosine kinase 3 (NTRK3) fusion nucleic acid molecule;
   sequencing the enriched sample, thereby producing a plurality of sequence reads;
   analyzing the plurality of sequence reads for the presence of the fusion nucleic acid molecule; and
   detecting, based on the analyzing step, the presence or absence of the fusion nucleic acid molecule in the sample from the individual.

Embodiment 40. The method of any one of embodiments 33-39, wherein the plurality of nucleic acid molecules comprises a mixture of cancer nucleic acid molecules and non-cancer nucleic acid molecules.

Embodiment 41. The method of embodiment 40, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Embodiment 42. The method of embodiment 40, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor, cell-free DNA (cfDNA) fraction or non-tumor blood cell fraction of the liquid biopsy sample.

Embodiment 43. The method of any one of embodiments 33-37 and 40-42, wherein the one or more adapters comprise amplification primers, flow cell adaptor sequences, substrate adapter sequences, or sample index sequences.

Embodiment 44. The method of any one of embodiments 39-43, wherein the selectively enriching comprises: (a) combining one or more bait molecules with the library, thereby hybridizing the one or more bait molecules to one or more nucleic acid molecules comprising nucleotide sequences corresponding to the fusion nucleic acid molecule and producing nucleic acid hybrids; and (b) isolating the nucleic acid hybrids to produce the enriched sample.

Embodiment 45. The method of any one of embodiments 33-37 and 40-43, wherein the captured nucleic acid molecules are captured from the amplified nucleic acid molecules by hybridization to one or more bait molecules.

Embodiment 46. The method of any one of embodiments 33-45, wherein the amplifying comprises performing a polymerase chain reaction (PCR) amplification technique, a non-PCR amplification technique, or an isothermal amplification technique.

Embodiment 47. The method of any one of embodiments 33-46, wherein the sequencing comprises use of a massively parallel sequencing (MPS) technique, whole genome sequencing (WGS), whole exome sequencing, targeted sequencing, direct sequencing, or a Sanger sequencing technique.

Embodiment 48. The method of embodiment 47, wherein the sequencing comprises a massively parallel sequencing technique, and the massively parallel sequencing technique comprises next generation sequencing (NGS).

Embodiment 49. The method of any one of embodiments 33-37, 40-43, and 45-48, wherein the sequencer comprises a next generation sequencer.

Embodiment 50. The method of any one of embodiments 33-49, further comprising generating a genomic profile for the individual, based, at least in part, on detecting the presence or absence of the fusion nucleic acid molecule.

Embodiment 51. The method of embodiment 50, wherein the genomic profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 52. The method of embodiment 50 or embodiment 51, wherein the genomic profile for the individual further comprises results from a nucleic acid sequencing-based test.

Embodiment 53. The method of any one of embodiments 50-52, further comprising selecting a treatment, administering a treatment, or applying a treatment to the individual based on the generated genomic profile, wherein the treatment comprises an anti-cancer therapy.

Embodiment 54. The method of any one of embodiments 33-53, further comprising generating a report indicating the presence or absence of the fusion nucleic acid molecule in the sample.

Embodiment 55. The method of embodiment 36 or embodiment 37, further comprising generating, by the one or more processors, a report indicating the presence or absence of the fusion nucleic acid molecule in the sample.

Embodiment 56. The method of embodiment 54 or embodiment 55, further comprising transmitting the report to a healthcare provider.

Embodiment 57. The method of embodiment 56, wherein the report is transmitted via a computer network or a peer-to-peer connection.

Embodiment 58. A method of identifying a candidate treatment for a cancer in an individual in need thereof, comprising performing DNA sequencing on a sample obtained from the individual to determine a sequencing mutation profile on a fusion nucleic acid molecule, wherein the sequencing mutation profile identifies the presence or absence of a fusion nucleic acid molecule, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule;
   wherein the candidate treatment comprises an anti-cancer therapy.

Embodiment 59. A method of identifying a candidate treatment for a cancer in an individual in need thereof, comprising performing DNA sequencing on a sample obtained from the individual to determine a sequencing mutation profile on a fusion nucleic acid molecule, wherein the sequencing mutation profile identifies the presence or absence of a fusion nucleic acid molecule, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule;
   wherein the candidate treatment comprises an anti-cancer therapy.

Embodiment 60. The method of embodiment 58 or embodiment 59, wherein the sequencing comprises use of a massively parallel sequencing (MPS) technique, whole genome sequencing (WGS), whole exome sequencing, targeted sequencing, direct sequencing, or a Sanger sequencing technique.

Embodiment 61. The method of embodiment 60, wherein the sequencing comprises a massively parallel sequencing technique, and the massively parallel sequencing technique comprises next generation sequencing (NGS).

Embodiment 62. The method of any one of embodiments 58-61, wherein the sequencing mutation profile identifies the presence or absence of a fragment of the fusion nucleic acid molecule comprising a breakpoint.

Embodiment 63. A method of treating or delaying progression of cancer, comprising:
detecting in a sample from an individual having a cancer a fusion nucleic acid molecule, or a fusion polypeptide encoded by the fusion nucleic acid molecule; wherein the fusion nucleic acid molecule is:
   (a) a neuropilin 2 (NRP2)-anaplastic lymphoma kinase (ALK) fusion nucleic acid molecule;
   (b) a phosphodiesterase 3A (PDE3A)-anaplastic lymphoma kinase (ALK) fusion nucleic acid molecule;
   (c) a proteasome 26S subunit, non-ATPase 14 (PSMD14)-anaplastic lymphoma kinase (ALK) fusion nucleic acid molecule;
   (d) an SFT2 domain containing 1 (SFT2D1)-anaplastic lymphoma kinase (ALK) fusion nucleic acid molecule;
   (e) a solute carrier family 37 member 3 (SLC37A3)-anaplastic lymphoma kinase (ALK) fusion nucleic acid molecule;
   (f) a transport and golgi organization 6 homolog (TANGO6)-anaplastic lymphoma kinase (ALK) fusion nucleic acid molecule; or
   (g) a WD repeat-containing protein 92 (WDR92)-anaplastic lymphoma kinase (ALK) fusion nucleic acid molecule; and
administering to the individual an effective amount of a treatment that comprises an anti-cancer therapy.

Embodiment 64. The method of any one of embodiments 1-10, 32, 53, 58, and 60-63, wherein the anti-cancer therapy comprises an ALK-targeted therapy.

Embodiment 65. The method of embodiment 64, wherein the ALK-targeted therapy comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof.

Embodiment 66. The method of any one of embodiments 1-10, 32, 53, 58, and 60-65, wherein the anti-cancer therapy comprises a kinase inhibitor.

Embodiment 67. The method of embodiment 66, wherein the kinase inhibitor is a multi-kinase inhibitor or an ALK-specific inhibitor.

Embodiment 68. The method of embodiment 66 or embodiment 67, wherein the kinase inhibitor is a tyrosine kinase inhibitor.

Embodiment 69. The method of any one of embodiments 66-68, wherein the kinase inhibitor inhibits kinase activity of an ALK polypeptide.

Embodiment 70. The method of any one of embodiments 66-69, wherein the kinase inhibitor is one or more of crizotinib, alectinib, ceritinib, lorlatinib, brigatinib, ensartinib (X-396), repotrectinib (TPX-0005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A.

Embodiment 71. The method of any one of embodiments 65-70, wherein the anti-cancer therapy comprises a cellular therapy, and wherein the cellular therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 72. The method of any one of embodiments 65-71, wherein the anti-cancer therapy comprises a nucleic acid that inhibits the expression of the fusion nucleic acid molecule or the fusion polypeptide encoded by the fusion nucleic acid molecule.

Embodiment 73. The method of any one of embodiments 65-72, wherein the anti-cancer therapy comprises a nucleic acid that comprises a double-stranded RNA (dsRNA), a small interfering RNA (siRNA), or a small hairpin RNA (shRNA).

Embodiment 74. The method of any one of embodiments 1-12, 25, 27-29, 31-33, 35-38, 40-58, and 60-73, wherein the fusion nucleic acid molecule is an NRP2-ALK fusion nucleic acid molecule.

Embodiment 75. The method of embodiment 74, wherein the fusion nucleic acid molecule comprises a 5′ breakpoint between exons 8 and 9 of NRP2.

Embodiment 76. The method of embodiment 74, wherein the fusion nucleic acid molecule comprises exons 1-8 of NRP2.

Embodiment 77. The method of any one of embodiments 74-76, wherein the fusion nucleic acid molecule comprises a 3′ breakpoint between exons 18 and 19 of ALK.

Embodiment 78. The method of any one of embodiments 74-76, wherein the fusion nucleic acid molecule comprises exons 19-29 of ALK.

Embodiment 79. The method of any one of embodiments 1-12, 25, 27-29, 31-33, 35-38, 40-58, and 60-73, wherein the fusion nucleic acid molecule is a PDE3A-ALK fusion nucleic acid molecule.

Embodiment 80. The method of embodiment 79, wherein the fusion nucleic acid molecule comprises a 5′ breakpoint between exons 10 and 11 of PDE3A.

Embodiment 81. The method of embodiment 79, wherein the fusion nucleic acid molecule comprises exons 1-10 of PDE3A.

Embodiment 82. The method of any one of embodiments 79-81, wherein the fusion nucleic acid molecule comprises a 3′ breakpoint between exons 7 and 8 of ALK.

Embodiment 83. The method of any one of embodiments 79-81, wherein the fusion nucleic acid molecule comprises exons 8-29 of ALK.

Embodiment 84. The method of any one of embodiments 1-12, 25, 27-29, 31-33, 35-38, 40-58, and 60-73, wherein the fusion nucleic acid molecule is a PSMD14-ALK fusion nucleic acid molecule.

Embodiment 85. The method of embodiment 84, wherein the fusion nucleic acid molecule comprises a 5′ breakpoint between exons 1 and 2 of PSMD14.

Embodiment 86. The method of embodiment 84, wherein the fusion nucleic acid molecule comprises exon 1 of PSMD14.

Embodiment 87. The method of any one of embodiments 84-86, wherein the fusion nucleic acid molecule comprises a 3′ breakpoint between exons 3 and 4 of ALK.

Embodiment 88. The method of any one of embodiments 84-86, wherein the fusion nucleic acid molecule comprises exons 4-29 of ALK.

Embodiment 89. The method of any one of embodiments 1-12, 25, 27-29, 31-33, 35-38, 40-58, and 60-73, wherein the fusion nucleic acid molecule is an SFT2D1-ALK fusion nucleic acid molecule.

Embodiment 90. The method of embodiment 89, wherein the fusion nucleic acid molecule comprises a 5′ breakpoint between exons 1 and 2 of SFT2D1.

Embodiment 91. The method of embodiment 89, wherein the fusion nucleic acid molecule comprises exon 1 of SFT2D1.

Embodiment 92. The method of any one of embodiments 89-91, wherein the fusion nucleic acid molecule comprises a 3′ breakpoint between exons 5 and 6 of ALK.

Embodiment 93. The method of any one of embodiments 89-91, wherein the fusion nucleic acid molecule comprises exons 6-29 of ALK.

Embodiment 94. The method of any one of embodiments 1-12, 25, 27-29, 31-33, 35-38, 40-58, and 60-73, wherein the fusion nucleic acid molecule is an SLC37A3-ALK fusion nucleic acid molecule.

Embodiment 95. The method of embodiment 94, wherein the fusion nucleic acid molecule comprises a 5′ breakpoint between exons 3 and 4 of SLC37A3.

Embodiment 96. The method of embodiment 94, wherein the fusion nucleic acid molecule comprises exons 1-3 of SLC37A3.

Embodiment 97. The method of any one of embodiments 94-96, wherein the fusion nucleic acid molecule comprises a 3′ breakpoint between exons 3 and 4 of ALK.

Embodiment 98. The method of any one of embodiments 94-96, wherein the fusion nucleic acid molecule comprises exons 4-29 of ALK.

Embodiment 99. The method of any one of embodiments 1-12, 25, 27-29, 31-33, 35-38, 40-58, and 60-73, wherein the fusion nucleic acid molecule is a TANGO6-ALK fusion nucleic acid molecule.

Embodiment 100. The method of embodiment 99, wherein the fusion nucleic acid molecule comprises a 5′ breakpoint between exons 1 and 2 of TANGO6.

Embodiment 101. The method of embodiment 99, wherein the fusion nucleic acid molecule comprises exon 1 of TANGO6.

Embodiment 102. The method of any one of embodiments 99-101, wherein the fusion nucleic acid molecule comprises a 3′ breakpoint between exons 1 and 2 of ALK.

Embodiment 103. The method of any one of embodiments 99-101, wherein the fusion nucleic acid molecule comprises exons 2-29 of ALK.

Embodiment 104. The method of any one of embodiments 1-12, 25, 27-29, 31-33, 35-38, 40-58, and 60-73, wherein the fusion nucleic acid molecule is a WDR92-ALK fusion nucleic acid molecule.

Embodiment 105. The method of embodiment 104, wherein the fusion nucleic acid molecule comprises a 5′ breakpoint between exons 7 and 8 of WDR92.

Embodiment 106. The method of embodiment 104, wherein the fusion nucleic acid molecule comprises exons 1-7 of WDR92.

Embodiment 107. The method of any one of embodiments 104-106, wherein the fusion nucleic acid molecule comprises a 3′ breakpoint between exons 1 and 2 of ALK.

Embodiment 108. The method of any one of embodiments 104-106, wherein the fusion nucleic acid molecule comprises exons 2-29 of ALK.

Embodiment 109. The method of any one of embodiments 1-12, 25, 27-29, 31-33, 35-38, 40-58, and 60-108, wherein the fusion nucleic acid molecule encodes a fusion polypeptide having ALK kinase activity.

Embodiment 110. A method of treating or delaying progression of cancer, comprising:
detecting in a sample from an individual having a cancer a fusion nucleic acid molecule, or a fusion polypeptide encoded by the fusion nucleic acid molecule; wherein the fusion nucleic acid molecule is:
   (a) a glycoprotein A33 (GPA33)-neurotrophic receptor tyrosine kinase 1 (NTRK1) fusion nucleic acid molecule;
   (b) a family with sequence similarity 19 (chemokine (C-C motif)-like, member A2) (FAM19A2)-neurotrophic receptor tyrosine kinase 1 (NTRK1) fusion nucleic acid molecule;
   (c) a cleavage and polyadenylation specific factor 6 (CPSF6)-neurotrophic receptor tyrosine kinase 1 (NTRK1) fusion nucleic acid molecule;
   (d) a SUN domain containing ossification factor (SUCO)-neurotrophic receptor tyrosine kinase 1 (NTRK1) fusion nucleic acid molecule;
   (e) a calcyclin binding protein (CACYBP)-neurotrophic receptor tyrosine kinase 1 (NTRK1) fusion nucleic acid molecule;
   (f) a zinc finger protein 382 (ZNF382)-neurotrophic receptor tyrosine kinase 1 (NTRK1) fusion nucleic acid molecule;
   (g) a nudE neurodevelopment protein 1 (NDE1)-neurotrophic receptor tyrosine kinase 3 (NTRK3) fusion nucleic acid molecule;
   (h) a DiGeorge syndrome critical region gene 5 (DGCR5)-neurotrophic receptor tyrosine kinase 3 (NTRK3) fusion nucleic acid molecule; or
   (i) a ubiquitin conjugating enzyme E2 Q2 pseudogene 1 (UBE2Q2P1)-neurotrophic receptor tyrosine kinase 3 (NTRK3) fusion nucleic acid molecule; and
administering to the individual an effective amount of a treatment that comprises an anti-cancer therapy.

Embodiment 111. The method of any one of embodiments 13-22, 32, 53, 59, 60-62, and 110, wherein the anti-cancer therapy comprises an NTRK1- or NTRK3-targeted therapy.

Embodiment 112. The method of any one of embodiments 13-22, 32, 53, 59-62, 74, and 111, wherein the anti-cancer therapy comprises a kinase inhibitor.

Embodiment 113. The method of embodiment 112, wherein the kinase inhibitor is a multi-kinase inhibitor, an NTRK1-specific inhibitor, or an NTRK3-specific inhibitor.

Embodiment 114. The method of embodiment 112 or embodiment 113, wherein the kinase inhibitor is a tyrosine kinase inhibitor.

Embodiment 115. The method of any one of embodiments 112-114, wherein the kinase inhibitor inhibits kinase activity of an NTRK1 or NTRK3 polypeptide.

Embodiment 116. The method of any one of embodiments 112-115, wherein the kinase inhibitor is one or more of AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), LOXO-195, a macrocyclic compound, ONO-5390556, oxindole 3, pegcantratinib (SNA-120), PHA-848125, PLX7486, a pyrazole derivative, a pyrazolo[1, 5a]pyrimidine, a pyridocarbazole, a pyridoquinazolinyl, a pyridotriazole, a pyrrolidinyl thiourea, a pyrrolidinyl urea, a pyrrolo[2, 3-d]pyrimidine, a quinazolinyl, repotrectinib, Ro 08-2750, a substituted pyrazolo[1,5a]pyrimidine, sitravatinib, SNA-125, tavilermide, thiazole 20h, ARRY-772, AZD7451, belizatinib, selitrectinib, crizotinib, ONO-7579, merestinib, ensartinib, TSR-011, MGCD516, altiratinib, cabozantinib, XL-184, DCC-2701, F17752, regorafenib, dovitinib, BMS-754807, ENMD-2076, BMS-777607, midostaurin, MK5108, PF-03814735, SNS-314, nintedanib, ponatinib, foretinib, AZD 1480, or VMD-928.

Embodiment 117. The method of any one of embodiments 112-115, wherein the kinase inhibitor is ARRY-470 or larotrectinib, AZ-23, danusertib (PHA-739358), entrectinib, lestaurtinib (CEP-701), AZD7451, belizatinib, selitrectinib, or crizotinib.

Embodiment 118. The method of any one of embodiments 111-117, wherein the anti-cancer therapy comprises a cellular therapy, and wherein the cellular therapy comprises an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 119. The method of any one of embodiments 111-118, wherein the anti-cancer therapy comprises a nucleic acid that inhibits the expression of the fusion nucleic acid molecule or the fusion polypeptide encoded by the fusion nucleic acid molecule.

Embodiment 120. The method of any one of embodiments 111-119, wherein the anti-cancer therapy comprises a nucleic acid that comprises a double-stranded RNA (dsRNA), a small interfering RNA (siRNA), or a small hairpin RNA (shRNA).

Embodiment 121. The method of any one of embodiments 1-10, 13-22, 32, 53, 58-74, and 110-120, wherein the anti-cancer therapy or the one or more treatment options further comprise an additional anti-cancer therapy.

Embodiment 122. The method of embodiment 121, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Embodiment 123. The method of any one of embodiments 13-24, 26-28, 30-32, 34-37, 39-57, 59-62, and 110-122, wherein the fusion nucleic acid molecule is a GPA33-NTRK1 fusion nucleic acid molecule.

Embodiment 124. The method of embodiment 123, wherein the fusion nucleic acid molecule comprises a 5′ breakpoint between exons 4 and 5 of GPA33.

Embodiment 125. The method of embodiment 123, wherein the fusion nucleic acid molecule comprises exons 1-4 of GPA33.

Embodiment 126. The method of any one of embodiments 123-125, wherein the fusion nucleic acid molecule comprises a 3′ breakpoint between exons 4 and 5 of NTRK1.

Embodiment 127. The method of any one of embodiments 123-125, wherein the fusion nucleic acid molecule comprises exons 5-17 of NTRK1.

Embodiment 128. The method of any one of embodiments 13-24, 26-28, 30-32, 34-37, 39-57, 59-62, and 110-122, wherein the fusion nucleic acid molecule is a FAM19A2-NTRK1 fusion nucleic acid molecule.

Embodiment 129. The method of embodiment 128, wherein the fusion nucleic acid molecule comprises a 5′ breakpoint between exons 1 and 2 of FAM19A2.

Embodiment 130. The method of embodiment 128, wherein the fusion nucleic acid molecule comprises exon 1 of FAM19A2.

Embodiment 131. The method of any one of embodiments 128-130, wherein the fusion nucleic acid molecule comprises a 3′ breakpoint between exons 3 and 4 of NTRK1.

Embodiment 132. The method of any one of embodiments 128-130, wherein the fusion nucleic acid molecule comprises exons 4-17 of NTRK1.

Embodiment 133. The method of any one of embodiments 13-24, 26-28, 30-32, 34-37, 39-57, 59-62, and 110-122, wherein the fusion nucleic acid molecule is a CPSF6-NTRK1 fusion nucleic acid molecule.

Embodiment 134. The method of embodiment 133, wherein the fusion nucleic acid molecule comprises a 5′ breakpoint between exons 7 and 8 of CPSF6.

Embodiment 135. The method of embodiment 133, wherein the fusion nucleic acid molecule comprises exons 1-7 of CPSF6.

Embodiment 136. The method of any one of embodiments 133-135, wherein the fusion nucleic acid molecule comprises a 3′ breakpoint between exons 11 and 12 of NTRK1.

Embodiment 137. The method of any one of embodiments 133-135, wherein the fusion nucleic acid molecule comprises exons 12-17 of NTRK1.

Embodiment 138. The method of any one of embodiments 13-24, 26-28, 30-32, 34-37, 39-57, 59-62, and 110-122, wherein the fusion nucleic acid molecule is a SUCO-NTRK1 fusion nucleic acid molecule.

Embodiment 139. The method of embodiment 138, wherein the fusion nucleic acid molecule comprises a 5′ breakpoint between exons 10 and 11 of SUCO.

Embodiment 140. The method of embodiment 138, wherein the fusion nucleic acid molecule comprises exons 1-10 of SUCO.

Embodiment 141. The method of any one of embodiments 138-140, wherein the fusion nucleic acid molecule comprises a 3′ breakpoint between exons 2 and 3 of NTRK1.

Embodiment 142. The method of any one of embodiments 138-140, wherein the fusion nucleic acid molecule comprises exons 3-17 of NTRK1.

Embodiment 143. The method of any one of embodiments 13-24, 26-28, 30-32, 34-37, 39-57, 59-62, and 110-122, wherein the fusion nucleic acid molecule is a CACYBP-NTRK1 fusion nucleic acid molecule.

Embodiment 144. The method of embodiment 143, wherein the fusion nucleic acid molecule comprises a 5′ breakpoint between exons 2 and 3 of CACYBP.

Embodiment 145. The method of embodiment 143, wherein the fusion nucleic acid molecule comprises exons 1 and 2 of CACYBP.

Embodiment 146. The method of any one of embodiments 143-145, wherein the fusion nucleic acid molecule comprises a 3′ breakpoint between exons 8 and 9 of NTRK1.

Embodiment 147. The method of any one of embodiments 143-145, wherein the fusion nucleic acid molecule comprises exons 9-17 of NTRK1.

Embodiment 148. The method of any one of embodiments 13-24, 26-28, 30-32, 34-37, 39-57, 59-62, and 110-122, wherein the fusion nucleic acid molecule is a ZNF382-NTRK1 fusion nucleic acid molecule.

Embodiment 149. The method of embodiment 148, wherein the fusion nucleic acid molecule comprises a 5′ breakpoint between exons 4 and 5 of ZNF382.

Embodiment 150. The method of embodiment 148, wherein the fusion nucleic acid molecule comprises exons 1-4 of ZNF382.

Embodiment 151. The method of any one of embodiments 148-150, wherein the fusion nucleic acid molecule comprises a 3′ breakpoint between exons 8 and 9 of NTRK1.

Embodiment 152. The method of any one of embodiments 148-150, wherein the fusion nucleic acid molecule comprises exons 9-17 of NTRK1.

Embodiment 153. The method of any one of embodiments 13-24, 26-28, 30-32, 34-37, 39-57, 59-62, and 110-152, wherein the fusion nucleic acid molecule encodes a fusion polypeptide having NTRK1 kinase activity.

Embodiment 154. The method of any one of embodiments 13-24, 26-28, 30-32, 34-37, 39-57, 59-62, and 110-122, wherein the fusion nucleic acid molecule is an NDE1-NTRK3 fusion nucleic acid molecule.

Embodiment 155. The method of embodiment 154, wherein the fusion nucleic acid molecule comprises a 5′ breakpoint between exons 6 and 7 of NDE1.

Embodiment 156. The method of embodiment 154, wherein the fusion nucleic acid molecule comprises exons 1-6 of NDE1.

Embodiment 157. The method of any one of embodiments 154-156, wherein the fusion nucleic acid molecule comprises a 3′ breakpoint between exons 13 and 14 of NTRK3.

Embodiment 158. The method of any one of embodiments 154-156, wherein the fusion nucleic acid molecule comprises exons 14-19 of NTRK3.

Embodiment 159. The method of any one of embodiments 13-24, 26-28, 30-32, 34-37, 39-57, 59-62, and 110-122, wherein the fusion nucleic acid molecule is a DGCR5-NTRK3 fusion nucleic acid molecule.

Embodiment 160. The method of embodiment 159, wherein the fusion nucleic acid molecule comprises a 5′ breakpoint between exons 1 and 2 of DGCR5.

Embodiment 161. The method of embodiment 159, wherein the fusion nucleic acid molecule comprises exon 1 of DGCR5.

Embodiment 162. The method of any one of embodiments 159-161, wherein the fusion nucleic acid molecule comprises a 3′ breakpoint between exons 2 and 3 of NTRK3.

Embodiment 163. The method of any one of embodiments 159-161, wherein the fusion nucleic acid molecule comprises exons 3-19 of NTRK3.

Embodiment 164. The method of any one of embodiments 13-24, 26-28, 30-32, 34-37, 39-57, 59-62, and 110-122, wherein the fusion nucleic acid molecule is a UBE2Q2P1-NTRK3 fusion nucleic acid molecule.

Embodiment 165. The method of embodiment 164, wherein the fusion nucleic acid molecule comprises a 5′ breakpoint between exons 5 and 6 of UBE2Q2P1.

Embodiment 166. The method of embodiment 164, wherein the fusion nucleic acid molecule comprises exons 1-5 of UBE2Q2P1.

Embodiment 167. The method of any one of embodiments 164-166, wherein the fusion nucleic acid molecule comprises a 3′ breakpoint between exons 5 and 6 of NTRK3.

Embodiment 168. The method of any one of embodiments 164-166, wherein the fusion nucleic acid molecule comprises exons 6-19 of NTRK3.

Embodiment 169. The method of any one of embodiments 13-24, 26-28, 30-32, 34-37, 39-57, 59-62, 110-120, and 154-168, wherein the fusion nucleic acid molecule encodes a fusion polypeptide having NTRK3 kinase activity.

Embodiment 170. The method of any one of embodiments 1-169, wherein the cancer is a sarcoma.

Embodiment 171. The method of embodiment 170, wherein the cancer is a uterus leiomyosarcoma, soft tissue inflammatory myofibroblastic tumor, soft tissue sarcoma (nos), bone osteosarcoma, soft tissue leiomyosarcoma, soft tissue sarcoma undifferentiated, soft tissue malignant peripheral nerve sheath tumor (mpnst), soft tissue liposarcoma, uterus sarcoma (nos), or soft tissue myxofibrosarcoma.

Embodiment 172. The method of any one of embodiments 1-171, further comprising obtaining the sample from the individual.

Embodiment 173. The method of any one of embodiments 1-172, wherein the sample is obtained from the cancer.

Embodiment 174. The method of any one of embodiments 1-172, wherein the sample comprises a tissue biopsy sample, a liquid biopsy sample, or a normal control.

Embodiment 175. The method of embodiment 174, wherein the sample is from a tumor biopsy, tumor specimen, or circulating tumor cell.

Embodiment 176. The method of any one of embodiments 1-172, wherein the sample is a liquid biopsy sample and comprises blood, plasma, cerebrospinal fluid, sputum, stool, urine, or saliva.

Embodiment 177. The method of any one of embodiments 1-176, wherein the sample comprises cells and/or nucleic acids from the cancer.

Embodiment 178. The method of embodiment 177, wherein the sample comprises mRNA, DNA, circulating tumor DNA (ctDNA), cell-free DNA, or cell-free RNA from the cancer.

Embodiment 179. The method of embodiment 176, wherein the sample is a liquid biopsy sample and comprises circulating tumor cells (CTCs).

Embodiment 180. The method of embodiment 176, wherein the sample is a liquid biopsy sample and comprises cell-free DNA (cfDNA), circulating tumor DNA (ctDNA), or any combination thereof.

Embodiment 181. The method of any one of embodiments 1-180, comprising acquiring knowledge of or detecting the fusion nucleic acid molecule or the fusion polypeptide encoded by the fusion nucleic acid molecule in a tissue biopsy sample, in a liquid biopsy sample, or in both a tissue biopsy sample and a liquid biopsy sample, from the individual.

Embodiment 182. The method of any one of embodiments 4-10 and 16-181, wherein the acquiring knowledge comprises detecting the fusion nucleic acid molecule or the fusion polypeptide encoded by the fusion nucleic acid molecule in the sample.

Embodiment 183. The method of any one of embodiments 1-3, 11-15, 23-57, and 63-182, wherein the detecting comprises detecting a fragment of the fusion nucleic acid molecule comprising a breakpoint or fusion junction.

Embodiment 184. The method of any one of embodiments 1-3, 11-15, 23-57, and 63-183, wherein the fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Embodiment 185. The method of embodiment 184, wherein the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

Embodiment 186. The method of any one of embodiments 1-3, 11-15, 23-57, and 63-182, wherein detecting the fusion polypeptide comprises detecting a portion of the fusion polypeptide that is encoded by a fragment of the fusion nucleic acid molecule that comprises a breakpoint or a fusion junction.

Embodiment 187. The method of any one of embodiments 1-3, 11-15, 23-57, 63-182, and 186, wherein the fusion polypeptide is detected in the sample by one or more of: immunoblotting, enzyme linked immunosorbent assay (ELISA), immunohistochemistry, or mass spectrometry.

Embodiment 188. The method of any one of embodiments 1-37 and 45-187, further comprising selectively enriching for one or more nucleic acids in the sample comprising nucleotide sequences corresponding to the fusion nucleic acid molecule; wherein the selectively enriching produces an enriched sample.

Embodiment 189. The method of embodiment 188, wherein the selectively enriching comprises: (a) combining one or more bait molecules with the sample, thereby hybridizing the one or more bait molecules to one or more nucleic acids in the sample comprising nucleotide sequences corresponding to the fusion nucleic acid molecule and producing nucleic acid hybrids; and (b) isolating the nucleic acid hybrids to produce the enriched sample.

Embodiment 190. The method of any one of embodiments 37 and 44-189, wherein the one or more bait molecules comprise a capture nucleic acid molecule configured to hybridize to a nucleotide sequence corresponding to the fusion nucleic acid molecule.

Embodiment 191. The method of embodiment 190, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Embodiment 192. The method of any one of embodiments 37 and 44-191, wherein the one or more bait molecules are conjugated to an affinity reagent or to a detection reagent.

Embodiment 193. The method of embodiment 192, wherein the affinity reagent is an antibody, an antibody fragment, or biotin, or wherein the detection reagent is a fluorescent marker.

Embodiment 194. The method of any one of embodiments 190-193, wherein the capture nucleic acid molecule comprises a DNA, RNA, or mixed DNA/RNA molecule.

Embodiment 195. The method of any one of embodiments 1-37 and 45-194, wherein the selectively enriching comprises amplifying the one or more nucleic acids comprising nucleotide sequences corresponding to the fusion nucleic acid molecule using a polymerase chain reaction (PCR) to produce an enriched sample.

Embodiment 196. The method of any one of embodiments 188-195, further comprising sequencing the enriched sample.

Embodiment 197. The method of any one of embodiments 1-196, wherein the individual is a human.

Embodiment 198. A kit comprising a probe or bait for detecting a fusion nucleic acid molecule, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 199. A kit comprising a probe or bait for detecting a fusion nucleic acid molecule, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 200. A nucleic acid molecule comprising a fusion nucleic acid molecule, or a fragment thereof comprising a breakpoint or fusion junction, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 201. A nucleic acid molecule comprising a fusion nucleic acid molecule, or a fragment thereof comprising a breakpoint or fusion junction, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 202. A vector comprising the nucleic acid molecule of embodiment 200 or embodiment 201.

Embodiment 203. A host cell comprising the vector of embodiment 202.

Embodiment 204. An antibody or antibody fragment that specifically binds to a fusion polypeptide, or to a portion thereof, encoded by a fusion nucleic acid molecule, or a fragment thereof comprising a breakpoint or fusion junction, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 205. An antibody or antibody fragment that specifically binds to a fusion polypeptide, or to a portion thereof, encoded by a fusion nucleic acid molecule, or a fragment thereof comprising a breakpoint or fusion junction, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 206. A kit comprising an antibody or antibody fragment for detecting a fusion polypeptide, or to a portion thereof, encoded by a fusion nucleic acid molecule, or a fragment thereof comprising a breakpoint or fusion junction, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 207. A kit comprising an antibody or antibody fragment for detecting a fusion polypeptide, or to a portion thereof, encoded by a fusion nucleic acid molecule, or a fragment thereof comprising a breakpoint or fusion junction, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 208. In vitro use of one or more oligonucleotides for detecting a fusion nucleic acid molecule, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 209. In vitro use of one or more oligonucleotides for detecting a fusion nucleic acid molecule, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 210. A kit comprising one or more oligonucleotides for detecting a fusion nucleic acid molecule, wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 211. A kit comprising one or more oligonucleotides for detecting a fusion nucleic acid molecule, wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 212. A system, comprising:
a memory configured to store one or more program instructions; and
one or more processors configured to execute the one or more program instructions, the one or more program instructions when executed by the one or more processors are configured to:
   (a) obtain a plurality of sequence reads of one or more nucleic acid molecules, wherein the one or more nucleic acid molecules are derived from a sample obtained from an individual;
   (b) analyze the plurality of sequence reads for the presence of a fusion nucleic acid molecule; and
   (c) detect, based on the analyzing, the fusion nucleic acid molecule in the sample; wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 213. A system, comprising:
a memory configured to store one or more program instructions; and
one or more processors configured to execute the one or more program instructions, the one or more program instructions when executed by the one or more processors are configured to:
   (a) obtain a plurality of sequence reads of one or more nucleic acid molecules, wherein the one or more nucleic acid molecules are derived from a sample obtained from an individual;
   (b) analyze the plurality of sequence reads for the presence of a fusion nucleic acid molecule; and
   (c) detect, based on the analyzing, the fusion nucleic acid molecule in the sample; wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 214. A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual;
(b) analyzing, using the one or more processors, the plurality of sequence reads for the presence of a fusion nucleic acid molecule; and
(c) detecting, using the one or more processors and based on the analyzing, the fusion nucleic acid molecule in the sample;
wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 215. A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual;
(b) analyzing, using the one or more processors, the plurality of sequence reads for the presence of a fusion nucleic acid molecule; and
(c) detecting, using the one or more processors and based on the analyzing, the fusion nucleic acid molecule in the sample;
wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 216. The system of embodiment 212 or embodiment 213, or the non-transitory computer readable storage medium of embodiment 214 or embodiment 215, wherein the sample is from an individual having a cancer.

Embodiment 217. The system or the non-transitory computer readable storage medium of embodiment 216, wherein the cancer is a sarcoma.

Embodiment 218. The system of any one of embodiments 212, 213, 216, or 217, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Embodiment 219. The non-transitory computer readable storage medium of any one of embodiments 214-217, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Embodiment 220. The system of any one of embodiments 212, 213, and 216-218, wherein the one or more program instructions when executed by the one or more processors are further configured to generate, based at least in part on the detecting, a genomic profile for the sample.

Embodiment 221. The non-transitory computer readable storage medium of any one of embodiments 214-217 and 219, wherein the method further comprises generating, based at least in part on the detecting, a genomic profile for the sample.

Embodiment 222. The system of embodiment 220, or the non-transitory computer readable storage medium of embodiment 221, wherein the individual is administered a treatment based at least in part on the genomic profile.

Embodiment 223. The system of embodiment 220 or embodiment 222, or the non-transitory computer readable storage medium of embodiment 221 or embodiment 222, wherein the genomic profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 224. The system of any one of embodiments 220, 222, and 223, or the non-transitory computer readable storage medium of any one of embodiments 221-223, wherein the genomic profile further comprises results from a nucleic acid sequencing-based test.

Embodiment 225. An anti-cancer therapy for use in a method of treating or delaying progression of cancer, wherein the method comprises administering the anti-cancer therapy to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 226. An anti-cancer therapy for use in a method of treating or delaying progression of cancer, wherein the method comprises administering the anti-cancer therapy to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

Embodiment 227. An anti-cancer therapy for use in the manufacture of a medicament for treating or delaying progression of cancer, wherein the medicament is to be administered to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule.

Embodiment 228. An anti-cancer therapy for use in the manufacture of a medicament for treating or delaying progression of cancer, wherein the medicament is to be administered to an individual, wherein a fusion nucleic acid molecule or a fragment thereof comprising a breakpoint or fusion junction, or a fusion polypeptide encoded by the fusion nucleic acid molecule, is detected in a sample obtained from the individual; wherein the nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule; (c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Fusion and Rearrangement Detection Using DNA and RNA-Based Comprehensive Genomic Profiling (CGP) of Sarcomas

Actionability of a growing subset of gene fusions and rearrangements (REs) is well established, with several alterations linked to approved targeted therapies. While there are FDA-approved assays for DNA-based detection of key recurring actionable RE, sarcomas can be enriched for rare REs that are not comprehensively covered on DNA panels.

In this Example, DNA and RNA CGP was performed on 9,969 sarcoma tissue specimens. DNA and RNA were co-extracted from 1.2 mm³ of FFPE tissue. Sequencing was performed for up to 406 cancer-related genes and introns from 28 genes commonly rearranged in cancer, as well as 265 RNA fusion genes.

Adaptor ligation-based hybrid capture-based sequencing was used. See, e.g., Frampton, G. M. et al. (2013) Nat. Biotech. 31:1023-1031. Mean coverage depth was >600×. Base substitutions, insertions, and deletions (short variants; SV) were detected. Actionable genes included NTRK1/2/3, BRAF MET, ALK, ERBB2, EGFR, FGFR1/2/3, ROS1, RET, and NRG1. Diagnostic genes/fusions included EWSR1, STAT6-NAB2, BCOR-ZC3H7B, BCOR-CCNB3, and FOXO1-PAX3/7. ALK fusions were breakpoints between intron 18 and 19 were considered canonical.

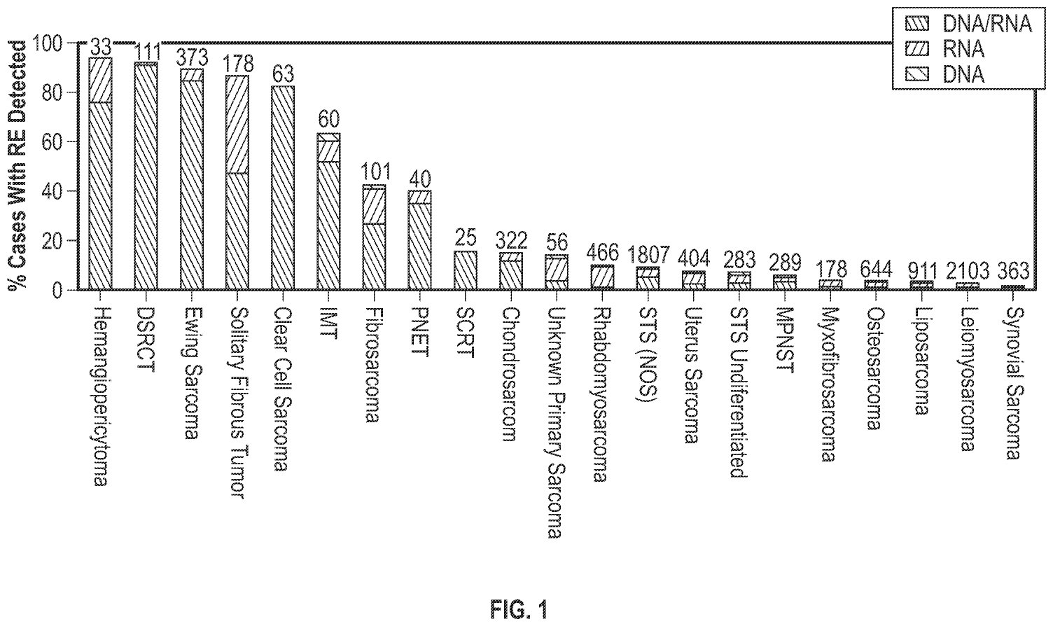

As shown in FIG. 1, detection of most REs in sarcoma occurred through DNA and RNA analysis. Exceptions were seen in gene fusions with rare breakpoints not covered by DNA baiting, particularly in hemangiopericytomas, solitary fibrous tumors, and rhabdomyosarcomas.

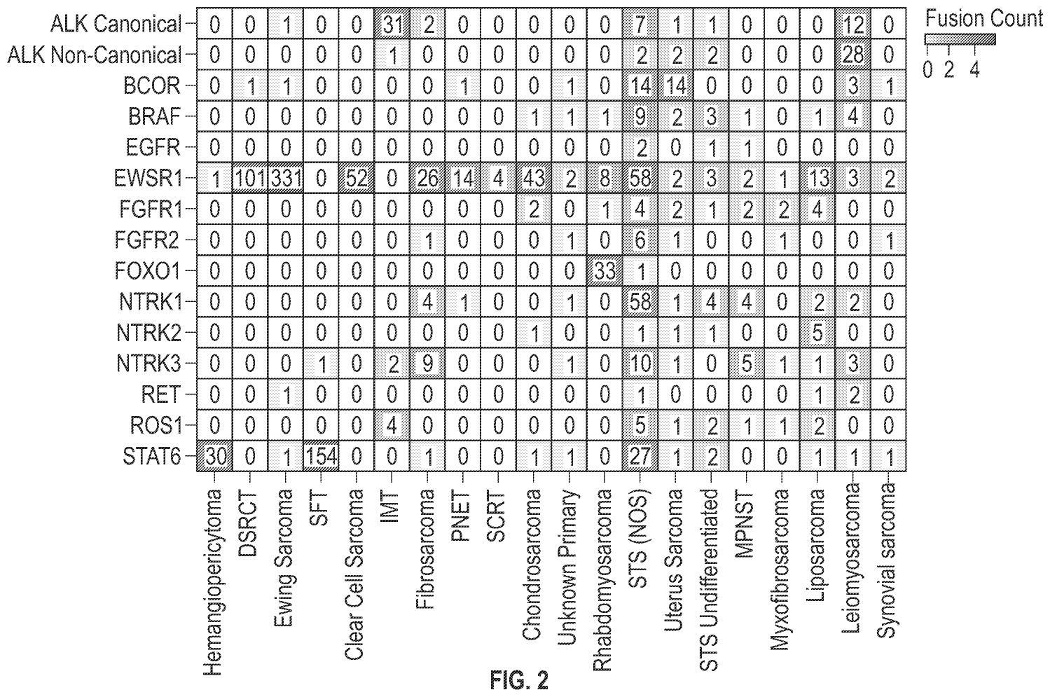

Diverse fusions were seen across a wide range of sarcomas. As shown in FIG. 2, 96% (892/927) of all fusions detected on DNA were confirmed in RNA. 25% (31/1271) of fusions were only detected in RNA, and 2% (30/1271) had complex DNA rearrangements resolved by analyzing RNA.

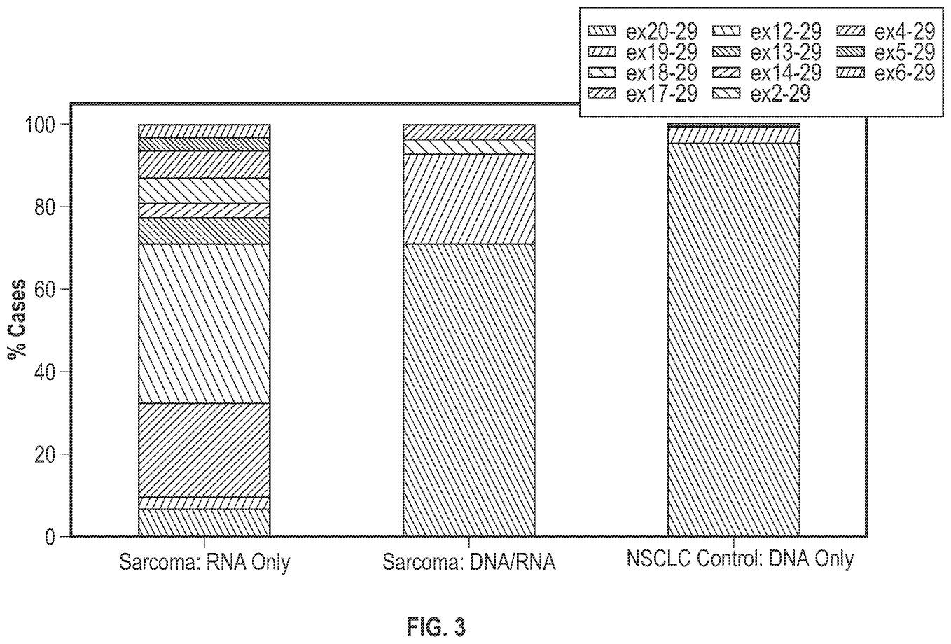

RNA analysis was able to detect ALK fusions with distinct breakpoints not covered by DNA baiting, which covers canonical NSCLC breakpoints (FIG. 3).

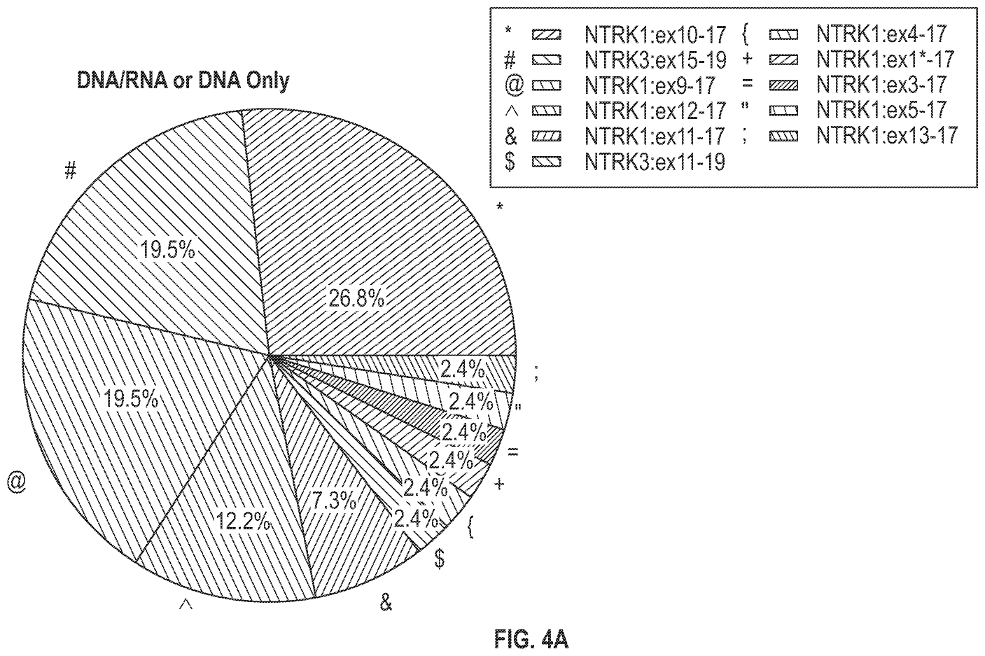

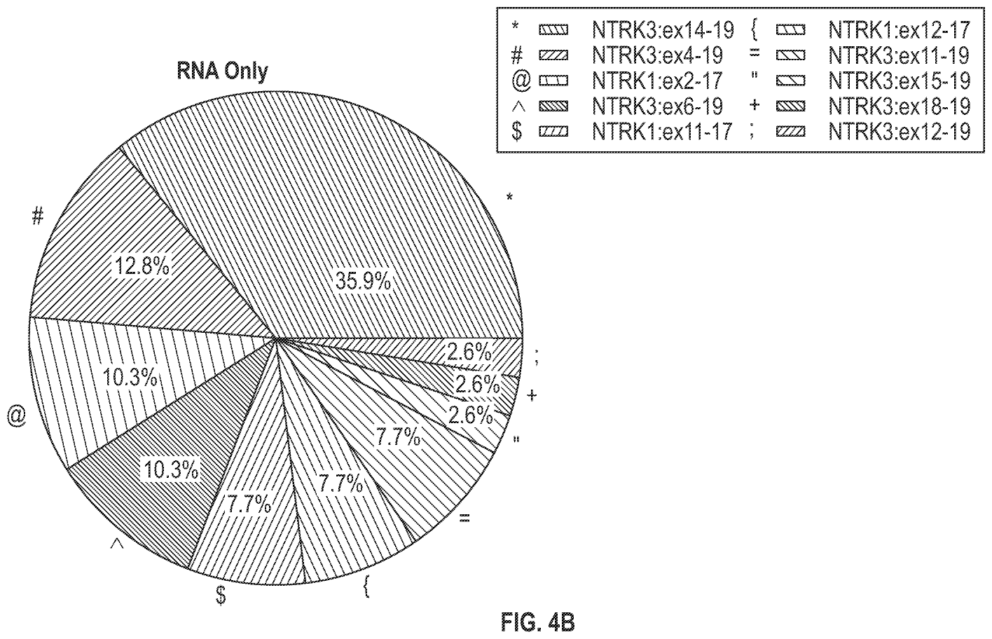

As shown in FIGS. 4A & 4B, of 41 NTRK1/3 gene fusions detected on DNA (5 DNA only; 36 DNA and RNA), 88% were confirmed in RNA. An additional 39 gene fusions were detected in RNA only; 100% were outside of the DNA baited region (NTRK1 intron 7, 8, and 9; NTRK3 no intron baiting).

These results demonstrated that, in most cases, rearrangements were detected in both DNA and RNA. However, RNA baiting increased the sensitivity for atypical fusions with non-canonical breakpoints. In ALK and NTRK1 non-fusion rearrangements, RNA further resolved the event as an actionable fusion or no RNA event was observed to support the actionability of the DNA finding.

1-62. (canceled)

63. A method of treating or delaying progression of cancer, comprising:
detecting in a sample from an individual having a cancer a fusion nucleic acid molecule, or a fusion polypeptide encoded by the fusion nucleic acid molecule; wherein the fusion nucleic acid molecule is:
(a) a neuropilin 2 (NRP2)-anaplastic lymphoma kinase (ALK) fusion nucleic acid molecule;
(b) a phosphodiesterase 3A (PDE3A)-anaplastic lymphoma kinase (ALK) fusion nucleic acid molecule;
(c) a proteasome 26S subunit, non-ATPase 14 (PSMD14)-anaplastic lymphoma kinase (ALK) fusion nucleic acid molecule;
(d) an SFT2 domain containing 1 (SFT2D1)-anaplastic lymphoma kinase (ALK) fusion nucleic acid molecule;
(e) a solute carrier family 37 member 3 (SLC37A3)-anaplastic lymphoma kinase (ALK) fusion nucleic acid molecule;
(f) a transport and golgi organization 6 homolog (TANGO6)-anaplastic lymphoma kinase (ALK) fusion nucleic acid molecule; or
(g) a WD repeat-containing protein 92 (WDR92)-anaplastic lymphoma kinase (ALK) fusion nucleic acid molecule; and
administering to the individual an effective amount of a treatment that comprises an anti-cancer therapy.

64. The method of claim 63, wherein the anti-cancer therapy comprises an ALK-targeted therapy that comprises a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof.

65-66. (canceled)

67. The method of claim 64, wherein the ALK-targeted therapy comprises a multi-kinase inhibitor, tyrosine kinase inhibitor, or an ALK-specific inhibitor.

68-69. (canceled)

70. The method of claim 67, wherein the ALK-targeted therapy comprises one or more of crizotinib, alectinib, ceritinib, lorlatinib, brigatinib, ensartinib (X-396), repotrectinib (TPX-0005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A.

71-73. (canceled)

74. The method of claim 63, wherein the fusion nucleic acid molecule is an NRP2-ALK fusion nucleic acid molecule that comprises:
(a) a 5′ breakpoint between exons 8 and 9 of NRP2;
(b) exons 1-8 of NRP2;
(c) a 3′ breakpoint between exons 18 and 19 of ALK; and/or
(d) exons 19-29 of ALK.

75-78. (canceled)

79. The method of claim 63, wherein the fusion nucleic acid molecule is a PDE3A-ALK fusion nucleic acid molecule that comprises:
(a) a 5′ breakpoint between exons 10 and 11 of PDE3A;
(b) exons 1-10 of PDE3A;
(c) a 3′ breakpoint between exons 7 and 8 of ALK; and/or
(d) exons 8-29 of ALK.

80-83. (canceled)

84. The method of claim 63, wherein the fusion nucleic acid molecule is a PSMD14-ALK fusion nucleic acid molecule that comprises:
(a) a 5′ breakpoint between exons 1 and 2 of PSMD14;
(b) exon 1 of PSMD14;
(c) a 3′ breakpoint between exons 3 and 4 of ALK; and/or
(d) exons 4-29 of ALK.

85-88. (canceled)

89. The method of claim 63, wherein the fusion nucleic acid molecule is an SFT2D1-ALK fusion nucleic acid molecule that comprises:
(a) a 5′ breakpoint between exons 1 and 2 of SFT2D1;
(b) exon 1 of SFT2D1;
(c) a 3′ breakpoint between exons 5 and 6 of ALK; and/or
(d) exons 6-29 of ALK.

90-93. (canceled)

94. The method of claim 63, wherein the fusion nucleic acid molecule is an SLC37A3-ALK fusion nucleic acid molecule that comprises:
(a) a 5′ breakpoint between exons 3 and 4 of SLC37A3;
(b) exons 1-3 of SLC37A3;
(c) a 3′ breakpoint between exons 3 and 4 of ALK; and/or
(d) exons 4-29 of ALK.

95-98. (canceled)

99. The method of claim 63, wherein the fusion nucleic acid molecule is a TANGO6-ALK fusion nucleic acid molecule that comprises:
(a) a 5′ breakpoint between exons 1 and 2 of TANGO6;
(b) exon 1 of TANGO6;
(c) a 3′ breakpoint between exons 1 and 2 of ALK; and/or
(d) exons 2-29 of ALK.

100-103. (canceled)

104. The method of claim 63, wherein the fusion nucleic acid molecule is a WDR92-ALK fusion nucleic acid molecule that comprises:
(a) a 5′ breakpoint between exons 7 and 8 of WDR92;
(b) exons 1-7 of WDR92;
(c) a 3′ breakpoint between exons 1 and 2 of ALK; and/or
(d) exons 2-29 of ALK.

105-109. (canceled)

110. A method of treating or delaying progression of cancer, comprising:
detecting in a sample from an individual having a cancer a fusion nucleic acid molecule, or a fusion polypeptide encoded by the fusion nucleic acid molecule; wherein the fusion nucleic acid molecule is:
(a) a glycoprotein A33 (GPA33)-neurotrophic receptor tyrosine kinase 1 (NTRK1) fusion nucleic acid molecule;
(b) a family with sequence similarity 19 (chemokine (C-C motif)-like, member A2) (FAM19A2)-neurotrophic receptor tyrosine kinase 1 (NTRK1) fusion nucleic acid molecule;
(c) a cleavage and polyadenylation specific factor 6 (CPSF6)-neurotrophic receptor tyrosine kinase 1 (NTRK1) fusion nucleic acid molecule;
(d) a SUN domain containing ossification factor (SUCO)-neurotrophic receptor tyrosine kinase 1 (NTRK1) fusion nucleic acid molecule;
(e) a calcyclin binding protein (CACYBP)-neurotrophic receptor tyrosine kinase 1 (NTRK1) fusion nucleic acid molecule;
(f) a zinc finger protein 382 (ZNF382)-neurotrophic receptor tyrosine kinase 1 (NTRK1) fusion nucleic acid molecule;
(g) a nudE neurodevelopment protein 1 (NDE1)-neurotrophic receptor tyrosine kinase 3 (NTRK3) fusion nucleic acid molecule;
(h) a DiGeorge syndrome critical region gene 5 (DGCR5)-neurotrophic receptor tyrosine kinase 3 (NTRK3) fusion nucleic acid molecule; or
(i) a ubiquitin conjugating enzyme E2 Q2 pseudogene 1 (UBE2Q2P1)-neurotrophic receptor tyrosine kinase 3 (NTRK3) fusion nucleic acid molecule; and
administering to the individual an effective amount of a treatment that comprises an anti-cancer therapy.

111. The method of claim 110, wherein the anti-cancer therapy comprises an NTRK1- or NTRK3-targeted therapy.

112. (canceled)

113. The method of claim 110, wherein the anti-cancer therapy comprises a multi-kinase inhibitor, an NTRK1-specific inhibitor, tyrosine kinase inhibitor, or an NTRK3-specific inhibitor.

114-115. (canceled)

116. The method of claim 110, wherein the kinase inhibitor is:
(a) one or more of AG 879 (Tyrphostin AG 879), an anti-TrK antibody, ARRY 954, AR523, AZ-23, AZ623, a benzotriazole, CEP-2563, danusertib (PHA-739358), entrectinib (also known as RXDX-101 or NMS-E628), DS-6051, GNF 5837, GW 441756, indenopyrrolocarboazole 12a, isothiazole 5n, larotrectinib (previously known as LOXO-101 or ARRY-470), lestaurtinib (CEP-701), LOXO-195, a macrocyclic compound, ONO-5390556, oxindole 3, pegcantratinib (SNA-120), PHA-848125, PLX7486, a pyrazole derivative, a pyrazolo[1, 5a]pyrimidine, a pyridocarbazole, a pyridoquinazolinyl, a pyridotriazole, a pyrrolidinyl thiourea, a pyrrolidinyl urea, a pyrrolo[2, 3-d]pyrimidine, a quinazolinyl, repotrectinib, Ro 08-2750, a substituted pyrazolo[1,5a]pyrimidine, sitravatinib, SNA-125, tavilermide, thiazole 20h, ARRY-772, AZD7451, belizatinib, selitrectinib, crizotinib, ONO-7579, merestinib, ensartinib, TSR-011, MGCD516, altiratinib, cabozantinib, XL-184, DCC-2701, F17752, regorafenib, dovitinib, BMS-754807, ENMD-2076, BMS-777607, midostaurin, MK5108, PF-03814735, SNS-314, nintedanib, ponatinib, foretinib, AZD 1480, or VMD-928, or
(b) ARRY-470 or larotrectinib, AZ-23, danusertib (PHA-739358), entrectinib, lestaurtinib (CEP-701), AZD7451, belizatinib, selitrectinib, or crizotinib.

117-122. (canceled)

123. The method of claim 110, wherein the fusion nucleic acid molecule is:
(a) a GPA33-NTRK1 fusion nucleic acid molecule that comprises: a 5′ breakpoint between exons 4 and 5 of GPA33, exons 1-4 of GPA33, a 3′ breakpoint between exons 4 and 5 of NTRK1, and/or exons 5-17 of NTRK;
(b) a FAM19A2-NTRK1 fusion nucleic acid molecule that comprises: a 5′ breakpoint between exons 1 and 2 of FAM19A2, exon 1 of FAM19A2, a 3′ breakpoint between exons 3 and 4 of NTRK1, and/or exons 4-17 of NTRK1;
(c) a CPSF6-NTRK1 fusion nucleic acid molecule that comprises: a 5′ breakpoint between exons 7 and 8 of CPSF6, exons 1-7 of CPSF6, a 3′ breakpoint between exons 11 and 12 of NTRK1, and/or exons 12-17 of NTRK1;
(d) a SUCO-NTRK1 fusion nucleic acid molecule that comprises: a 5′ breakpoint between exons 10 and 11 of SUCO, exons 1-10 of SUCO, a 3′ breakpoint between exons 2 and 3 of NTRK1, and/or exons 3-17 of NTRK1;
(e) a CACYBP-NTRK1 fusion nucleic acid molecule that comprises: a 5′ breakpoint between exons 2 and 3 of CACYBP, exons 1 and 2 of CACYBP, a 3′ breakpoint between exons 8 and 9 of NTRK1, and/or exons 9-17 of NTRK1; or
(f) a ZNF382-NTRK1 fusion nucleic acid molecule that comprises: a 5′ breakpoint between exons 4 and 5 of ZNF382, exons 1-4 of ZNF382, a 3′ breakpoint between exons 8 and 9 of NTRK1, and/or exons 9-17 of NTRK1.

124-153. (canceled)

154. The method of claim 110, wherein the fusion nucleic acid molecule is:
(a) an NDE1-NTRK3 fusion nucleic acid molecule that comprises, a 5′ breakpoint between exons 6 and 7 of NDE1, exons 1-6 of NDE1, a 3′ breakpoint between exons 13 and 14 of NTRK3, and/or exons 14-19 of NTRK3;
(b) a DGCR5-NTRK3 fusion nucleic acid molecule that comprises: a 5′ breakpoint between exons 1 and 2 of DGCR5, exon 1 of DGCR5, a 3′ breakpoint between exons 2 and 3 of NTRK3, and/or exons 3-19 of NTRK3; or
(c) a UBE2Q2P1-NTRK3 fusion nucleic acid molecule that comprises: a 5′ breakpoint between exons 5 and 6 of UBE2Q2P1, exons 1-5 of UBE2Q2P1, a 3′ breakpoint between exons 5 and 6 of NTRK3, and/or exons 6-19 of NTRK3.

155-169. (canceled)

170. The method of claim 63, wherein the cancer is a sarcoma.

171-176. (canceled)

177. The method of claim 63, wherein the sample;
(a) comprises cells and/or nucleic acids from the cancer;
(b) is a liquid biopsy sample and comprises circulating tumor cells (CTCs); or
(c) is a liquid biopsy sample and comprises cell-free DNA (cfDNA), circulating tumor DNA (ctDNA), or any combination thereof.

178-196. (canceled)

197. The method of claim 63, wherein the individual is a human.

198-228. (canceled)
